(19) United States
(12) Patent Application Publication
(10) Pub. No.: US 2026/0347496 A1
(43) Pub. Date: Nov. 5, 2026

(54) ANTIBODIES AGAINST CD24 AND USES THEREOF

(71) Applicant: Beijing neoX Biotech Limited, Beijing (CN)

(72) Inventors: Junli Liu, Shanghai (CN); Puwei Yuan, Beijing (CN); Ying Lei, Shanghai (CN); Mingchen Chen, Beijing (CN); Bingshi Guo, Shanghai (CN); Fan Liu, Beijing (CN)

(21) Appl. No.: 19/124,274

(22) PCT Filed: Oct. 26, 2023
(86) PCT No.: PCT/CN2023/126795

§ 371 Date: Apr. 24, 2025

(30) Foreign Application Priority Data
Oct. 27, 2022 (WO) PCT/CN2022/128003
Oct. 28, 2022 (WO) PCT/CN2022/128297
Sep. 26, 2023 (WO) PCT/CN2023/121689

(51) Int. Cl. C07K 16/28; A61K 39/00; A61P 35/00

(52) U.S. Cl. CPC C07K 16/2896; A61P 35/00; A61K 2039/505; C07K 2317/24; C07K 2317/622; C07K 2317/92

(57) ABSTRACT

Described herein are CD24 binding antibodies and fragments thereof.

### CROSS-REFERENCE

This patent application claims the benefit of International Application No. PCT/CN2022/128003, filed Oct. 27, 2022; International Application No. PCT/CN2022/128297, filed Oct. 28, 2022; and International Application No. PCT/CN2023/121689, filed Sep. 26, 2023; which are incorporated herein by reference in their entirety.

### BACKGROUND

CD24 is a highly glycosylated protein with a small protein core that is linked to the plasma membrane via a glycosyl-phosphatidylinositol anchor. CD24 is primarily expressed by immune cells but is often overexpressed in human tumors. In cancer, CD24 is a regulator of cell migration, invasion and proliferation. Thus, CD24 makes an attractive target for monocolonal antibodies as a cancer therapy.

### SUMMARY

Described herein are antibodies that specifically bind to CD24. The antibodies described herein possess the useful properties of not binding to either B cells or activated T cells. Such antibodies may possess an enhanced safety profile with reduced immune side effects.

Described herein in one aspect is an antibody, or antigen binding fragment thereof, that binds to CD24, wherein the antibody does not bind to T lymphocytes. In certain embodiments, the antibody does not bind to activated T lymphocytes. In certain embodiments, the antibody does not bind to B lymphocytes. In certain embodiments, the antibody binds to CD24 with a K_{D} of less than 1×10⁻⁸. In certain embodiments, the antibody binds to CD24 with a K_{D} of less than 1×10⁻⁹. In certain embodiments, the antibody is a humanized or chimeric antibody. In certain embodiments, the antibody is a Fab, F (ab) 2, or a single chain variable fragment (scFv). In certain embodiments, the antibody is a heavy chain antibody or antigen binding fragment thereof. In certain embodiments, the antibody is an IgG antibody. In certain embodiments, the antibody is an IgG₁ isotype. In certain embodiments, the antibody is an IgG₄ isotype. In certain embodiments, the antibody binds to the serine at residue 18 of SEQ ID NO: 500. In certain embodiments, the antibody requires the presence of a serine at residue 18 of SEQ ID NO: 500 for binding. In certain embodiments, the antibody binds to an epitope consisting of the amino acid sequence of SEQ ID NO 501.

Also described herein in another aspect is an antibody or antigen binding fragment thereof, that binds to CD24, wherein the antibody or antigen binding fragment thereof comprises: (a) a light chain complementarity determining region 1 (LCDR1) comprising an amino acid sequence set forth in SEQ ID NO: 401; (b) a light chain complementarity determining region 2 (LCDR2) comprising an amino acid sequence set forth in SEQ ID NO: 403; (c) a light chain complementarity determining region 3 (LCDR3) comprising an amino acid sequence set forth in SEQ ID NO: 405; (d) a heavy chain complementarity determining region 1 (HCDR1) comprising an amino acid sequence set forth in SEQ ID NO: 407; (e) a heavy chain complementarity determining region 2 (HCDR2) comprising an amino acid sequence set forth in SEQ ID NO: 409; and/or (f) a heavy chain complementarity determining region 3 (HCDR3) comprising an amino acid sequence set forth in SEQ ID NO: 411. In certain embodiments, the antibody comprises: (a) a light chain complementarity determining region 1 (LCDR1) comprising an amino acid sequence set forth in SEQ ID NO: 401; (b) a light chain complementarity determining region 2 (LCDR2) comprising an amino acid sequence set forth in SEQ ID NO: 403; (c) a light chain complementarity determining region 3 (LCDR3) comprising an amino acid sequence set forth in SEQ ID NO: 405; (d) a heavy chain complementarity determining region 1 (HCDR1) comprising an amino acid sequence set forth in SEQ ID NO: 407; (e) a heavy chain complementarity determining region 2 (HCDR2) comprising an amino acid sequence set forth in SEQ ID NO: 409; and/or (f) a heavy chain complementarity determining region 3 (HCDR3) comprising an amino acid sequence set forth in SEQ ID NO: 411. In certain embodiments, the antibody comprises: (a) a light chain complementarity determining region 1 (LCDR1) comprising an amino acid sequence set forth in SEQ ID NO: 402; (b) a light chain complementarity determining region 2 (LCDR2) comprising an amino acid sequence set forth in SEQ ID NO: 403; (c) a light chain complementarity determining region 3 (LCDR3) comprising an amino acid sequence set forth in SEQ ID NO: 406; (d) a heavy chain complementarity determining region 1 (HCDR1) comprising an amino acid sequence set forth in SEQ ID NO: 408; (e) a heavy chain complementarity determining region 2 (HCDR2) comprising an amino acid sequence set forth in SEQ ID NO: 409; and/or (f) a heavy chain complementarity determining region 3 (HCDR3) comprising an amino acid sequence set forth in SEQ ID NO: 412. In certain embodiments, the antibody comprises: (a) a light chain complementarity determining region 1 (LCDR1) as set forth in SEQ ID NO: 402; (b) a light chain complementarity determining region 2 (LCDR2) as set forth in SEQ ID NO: 403; (c) a light chain complementarity determining region 3 (LCDR3) as set forth in SEQ ID NO: 406; (d) a heavy chain complementarity determining region 1 (HCDR1) as set forth in SEQ ID NO: 408; (e) a heavy chain complementarity determining region 2 (HCDR2) as set forth in SEQ ID NO: 409; and/or (f) a heavy chain complementarity determining region 3 (HCDR3) as set forth in SEQ ID NO: 412. In certain embodiments, the antibody comprises: (a) a light chain complementarity determining region 1 (LCDR1) as set forth in SEQ ID NO: 402; (b) a light chain complementarity determining region 2 (LCDR2) as set forth in SEQ ID NO: 403; (c) a light chain complementarity determining region 3 (LCDR3) as set forth in SEQ ID NO: 406; (d) a heavy chain complementarity determining region 1 (HCDR1) as set forth in SEQ ID NO: 408; (e) a heavy chain complementarity determining region 2 (HCDR2) as set forth in SEQ ID NO: 409; and/or (f) a heavy chain complementarity determining region 3 (HCDR3) as set forth in SEQ ID NO: 412; and wherein the antibody does not bind to, or does not substantially bind to either B cells or activated T cells. In certain embodiments, the antibody comprises: (a) a light chain complementarity determining region 1 (LCDR1) comprising an amino acid sequence selected from the group consisting of SEQ ID NO: 13, 23, 33, 43, 53, 63, 73, 83, 703, 713, 723, 733, and 743; (b) a light chain complementarity determining region 2 (LCDR2) comprising an amino acid sequence selected from the group consisting of SEQ ID NO: 14, 24, 34, 44, 54, 64, 74, 84, 704, 714, 724, 734, and 744; (c) a light chain complementarity determining region 3 (LCDR3) comprising an amino acid sequence selected from the group consisting of SEQ ID NO: 15, 25, 35, 45, 55, 65, 75, 85, 705, 715, 725, 735, and 745; (d) a heavy chain complementarity determining region 1 (HCDR1) comprising an amino acid sequence selected from the group consisting of SEQ ID NO: 16, 26, 36, 46, 56, 66, 76, 86, 706, 716, 726, 736, and 746; (e) a heavy chain complementarity determining region 2 (HCDR2) comprising an amino acid sequence selected from the group consisting of SEQ ID NO: 17, 27, 37, 47, 57, 67, 77, 87, 707, 717, 727, 737, and 747; and/or (f) a heavy chain complementarity determining region 3 (HCDR3) comprising an amino acid sequence selected from the group consisting of SEQ ID NO: 18, 28, 38, 48, 58, 68, 78, 88, 708, 718, 728, 738, and 748. In certain embodiments, the antibody comprises a combination of LCDR1, LCDR2, and LCDR3 and HCDR1, HCDR2, and HCDR3, wherein the combination is selected from the group consisting of: (i) LCDR1 comprising SEQ ID NO: 13, LCDR2 comprising SEQ ID NO: 14, LCDR3 comprising SEQ ID NO: 15, HCDR1 comprising SEQ ID NO: 16, HCDR2 comprising SEQ ID NO: 17, HCDR3 comprising SEQ ID NO: 18; (ii) LCDR1 comprising SEQ ID NO: 23, LCDR2 comprising SEQ ID NO: 24, LCDR3 comprising SEQ ID NO: 25, HCDR1 comprising SEQ ID NO: 26, HCDR2 comprising SEQ ID NO: 27, HCDR3 comprising SEQ ID NO: 28; (iii) LCDR1 comprising SEQ ID NO: 33, LCDR2 comprising SEQ ID NO: 34, LCDR3 comprising SEQ ID NO: 35, HCDR1 comprising SEQ ID NO: 36, HCDR2 comprising SEQ ID NO: 37, HCDR3 comprising SEQ ID NO: 38; (iv) LCDR1 comprising SEQ ID NO: 43, LCDR2 comprising SEQ ID NO: 44, LCDR3 comprising SEQ ID NO: 45, HCDR1 comprising SEQ ID NO: 46, HCDR2 comprising SEQ ID NO: 47, HCDR3 comprising SEQ ID NO: 48; (v) LCDR1 comprising SEQ ID NO: 53, LCDR2 comprising SEQ ID NO: 54, LCDR3 comprising SEQ ID NO: 55, HCDR1 comprising SEQ ID NO: 56, HCDR2 comprising SEQ ID NO: 57, HCDR3 comprising SEQ ID NO: 58; (vi) LCDR1 comprising SEQ ID NO: 63, LCDR2 comprising SEQ ID NO: 64, LCDR3 comprising SEQ ID NO: 65, HCDR1 comprising SEQ ID NO: 66, HCDR2 comprising SEQ ID NO: 67, HCDR3 comprising SEQ ID NO: 68; (vii) LCDR1 comprising SEQ ID NO: 73, LCDR2 comprising SEQ ID NO: 74, LCDR3 comprising SEQ ID NO: 75, HCDR1 comprising SEQ ID NO: 76, HCDR2 comprising SEQ ID NO: 77, HCDR3 comprising SEQ ID NO: 78; (viii) LCDR1 comprising SEQ ID NO: 83, LCDR2 comprising SEQ ID NO: 84, LCDR3 comprising SEQ ID NO: 85, HCDR1 comprising SEQ ID NO: 86, HCDR2 comprising SEQ ID NO: 87, HCDR3 comprising SEQ ID NO: 88; (ix) LCDR1 comprising SEQ ID NO: 703, LCDR2 comprising SEQ ID NO: 704, LCDR3 comprising SEQ ID NO: 705, HCDR1 comprising SEQ ID NO: 706, HCDR2 comprising SEQ ID NO: 707, HCDR3 comprising SEQ ID NO: 708; (x) LCDR1 comprising SEQ ID NO: 713, LCDR2 comprising SEQ ID NO: 714, LCDR3 comprising SEQ ID NO: 715, HCDR1 comprising SEQ ID NO: 716, HCDR2 comprising SEQ ID NO: 717, HCDR3 comprising SEQ ID NO: 718; (xi) LCDR1 comprising SEQ ID NO: 723, LCDR2 comprising SEQ ID NO: 724, LCDR3 comprising SEQ ID NO: 725, HCDR1 comprising SEQ ID NO: 726, HCDR2 comprising SEQ ID NO: 727, HCDR3 comprising SEQ ID NO: 728; (xii) LCDR1 comprising SEQ ID NO: 733, LCDR2 comprising SEQ ID NO: 734, LCDR3 comprising SEQ ID NO: 735, HCDR1 comprising SEQ ID NO: 736, HCDR2 comprising SEQ ID NO: 737, HCDR3 comprising SEQ ID NO: 738; and (xiii) LCDR1 comprising SEQ ID NO: 743, LCDR2 comprising SEQ ID NO: 744, LCDR3 comprising SEQ ID NO: 745, HCDR1 comprising SEQ ID NO: 746, HCDR2 comprising SEQ ID NO: 747, HCDR3 comprising SEQ ID NO: 748. In certain embodiments, the antibody comprises a combination of LCDR1, LCDR2, and LCDR3 and HCDR1, HCDR2, and HCDR3, wherein the combination is selected from the group consisting of: (i) LCDR1 as set forth in SEQ ID NO: 13, LCDR2 as set forth in SEQ ID NO: 14, LCDR3 as set forth in SEQ ID NO: 15, HCDR1 as set forth in SEQ ID NO: 16, HCDR2 as set forth in SEQ ID NO: 17, HCDR3 as set forth in SEQ ID NO: 18; (ii) LCDR1 as set forth in SEQ ID NO: 23, LCDR2 as set forth in SEQ ID NO: 24, LCDR3 as set forth in SEQ ID NO: 25, HCDR1 as set forth in SEQ ID NO: 26, HCDR2 as set forth in SEQ ID NO: 27, HCDR3 as set forth in SEQ ID NO: 28; (iii) LCDR1 as set forth in SEQ ID NO: 33, LCDR2 as set forth in SEQ ID NO: 34, LCDR3 as set forth in SEQ ID NO: 35, HCDR1 as set forth in SEQ ID NO: 36, HCDR2 as set forth in SEQ ID NO: 37, HCDR3 as set forth in SEQ ID NO: 38; (iv) LCDR1 as set forth in SEQ ID NO: 43, LCDR2 as set forth in SEQ ID NO: 44, LCDR3 as set forth in SEQ ID NO: 45, HCDR1 as set forth in SEQ ID NO: 46, HCDR2 as set forth in SEQ ID NO: 47, HCDR3 as set forth in SEQ ID NO: 48; (v) LCDR1 as set forth in SEQ ID NO: 53, LCDR2 as set forth in SEQ ID NO: 54, LCDR3 as set forth in SEQ ID NO: 55, HCDR1 as set forth in SEQ ID NO: 56, HCDR2 as set forth in SEQ ID NO: 57, HCDR3 as set forth in SEQ ID NO: 58; (vi) LCDR1 as set forth in SEQ ID NO: 63, LCDR2 as set forth in SEQ ID NO: 64, LCDR3 as set forth in SEQ ID NO: 65, HCDR1 as set forth in SEQ ID NO: 66, HCDR2 as set forth in SEQ ID NO: 67, HCDR3 as set forth in SEQ ID NO: 68; (vii) LCDR1 as set forth in SEQ ID NO: 73, LCDR2 as set forth in SEQ ID NO: 74, LCDR3 as set forth in SEQ ID NO: 75, HCDR1 as set forth in SEQ ID NO: 76, HCDR2 as set forth in SEQ ID NO: 77, HCDR3 as set forth in SEQ ID NO: 78; (viii) LCDR1 as set forth in SEQ ID NO: 83, LCDR2 as set forth in SEQ ID NO: 84, LCDR3 as set forth in SEQ ID NO: 85, HCDR1 as set forth in SEQ ID NO: 86, HCDR2 as set forth in SEQ ID NO: 87, HCDR3 as set forth in SEQ ID NO: 88; (ix) LCDR1 as set forth in SEQ ID NO: 703, LCDR2 as set forth in SEQ ID NO: 704, LCDR3 as set forth in SEQ ID NO: 705, HCDR1 as set forth in SEQ ID NO: 706, HCDR2 as set forth in SEQ ID NO: 707, HCDR3 as set forth in SEQ ID NO: 708; (x) LCDR1 as set forth in SEQ ID NO: 713, LCDR2 as set forth in SEQ ID NO: 714, LCDR3 as set forth in SEQ ID NO: 715, HCDR1 as set forth in SEQ ID NO: 716, HCDR2 as set forth in SEQ ID NO: 717, HCDR3 as set forth in SEQ ID NO: 718; (xi) LCDR1 as set forth in SEQ ID NO: 723, LCDR2 as set forth in SEQ ID NO: 724, LCDR3 as set forth in SEQ ID NO: 725, HCDR1 as set forth in SEQ ID NO: 726, HCDR2 as set forth in SEQ ID NO: 727, HCDR3 as set forth in SEQ ID NO: 728; (xii) LCDR1 as set forth in SEQ ID NO: 733, LCDR2 as set forth in SEQ ID NO: 734, LCDR3 as set forth in SEQ ID NO: 735, HCDR1 as set forth in SEQ ID NO: 736, HCDR2 as set forth in SEQ ID NO: 737, HCDR3 as set forth in SEQ ID NO: 738; and (xiii) LCDR1 as set forth in SEQ ID NO: 743, LCDR2 as set forth in SEQ ID NO: 744, LCDR3 as set forth in SEQ ID NO: 745, HCDR1 as set forth in SEQ ID NO: 746, HCDR2 as set forth in SEQ ID NO: 747, HCDR3 as set forth in SEQ ID NO: 748. In certain embodiments, the antibody comprises: (a) a light chain complementarity determining region 1 (LCDR1) comprising an amino acid sequence set forth in SEQ ID NO: 13; (b) a light chain complementarity determining region 2 (LCDR2) comprising an amino acid sequence set forth in SEQ ID NO: 14; (c) a light chain complementarity determining region 3 (LCDR3) comprising an amino acid sequence set forth in SEQ ID NO: 15; (d) a heavy chain complementarity determining region 1 (HCDR1) comprising an amino acid sequence set forth in SEQ ID NO: 16; (e) a heavy chain complementarity determining region 2 (HCDR2) comprising an amino acid sequence set forth in SEQ ID NO: 17; and/or (f) a heavy chain complementarity determining region 3 (HCDR3) comprising an amino acid sequence set forth in SEQ ID NO: 18. In certain embodiments, the antibody comprises: (a) a light chain complementarity determining region 1 (LCDR1) comprising an amino acid sequence set forth in SEQ ID NO: 23; (b) a light chain complementarity determining region 2 (LCDR2) comprising an amino acid sequence set forth in SEQ ID NO: 24; (c) a light chain complementarity determining region 3 (LCDR3) comprising an amino acid sequence set forth in SEQ ID NO: 25; (d) a heavy chain complementarity determining region 1 (HCDR1) comprising an amino acid sequence set forth in SEQ ID NO: 26; (e) a heavy chain complementarity determining region 2 (HCDR2) comprising an amino acid sequence set forth in SEQ ID NO: 27; and/or (f) a heavy chain complementarity determining region 3 (HCDR3) comprising an amino acid sequence set forth in SEQ ID NO: 28. In certain embodiments, the antibody comprises: (a) a light chain complementarity determining region 1 (LCDR1) comprising an amino acid sequence set forth in SEQ ID NO: 33; (b) a light chain complementarity determining region 2 (LCDR2) comprising an amino acid sequence set forth in SEQ ID NO: 34; (c) a light chain complementarity determining region 3 (LCDR3) comprising an amino acid sequence set forth in SEQ ID NO: 35; (d) a heavy chain complementarity determining region 1 (HCDR1) comprising an amino acid sequence set forth in SEQ ID NO: 36; (e) a heavy chain complementarity determining region 2 (HCDR2) comprising an amino acid sequence set forth in SEQ ID NO: 37; and/or (f) a heavy chain complementarity determining region 3 (HCDR3) comprising an amino acid sequence set forth in SEQ ID NO: 38. In certain embodiments, the antibody comprises: (a) a light chain complementarity determining region 1 (LCDR1) comprising an amino acid sequence set forth in SEQ ID NO: 43; (b) a light chain complementarity determining region 2 (LCDR2) comprising an amino acid sequence set forth in SEQ ID NO: 44; (c) a light chain complementarity determining region 3 (LCDR3) comprising an amino acid sequence set forth in SEQ ID NO: 45; (d) a heavy chain complementarity determining region 1 (HCDR1) comprising an amino acid sequence set forth in SEQ ID NO: 46; (e) a heavy chain complementarity determining region 2 (HCDR2) comprising an amino acid sequence set forth in SEQ ID NO: 47; and/or (f) a heavy chain complementarity determining region 3 (HCDR3) comprising an amino acid sequence set forth in SEQ ID NO: 48. In certain embodiments, the antibody comprises: (a) a light chain complementarity determining region 1 (LCDR1) comprising an amino acid sequence set forth in SEQ ID NO: 53; (b) a light chain complementarity determining region 2 (LCDR2) comprising an amino acid sequence set forth in SEQ ID NO: 54; (c) a light chain complementarity determining region 3 (LCDR3) comprising an amino acid sequence set forth in SEQ ID NO: 55; (d) a heavy chain complementarity determining region 1 (HCDR1) comprising an amino acid sequence set forth in SEQ ID NO: 56; (e) a heavy chain complementarity determining region 2 (HCDR2) comprising an amino acid sequence set forth in SEQ ID NO: 57; and/or (f) a heavy chain complementarity determining region 3 (HCDR3) comprising an amino acid sequence set forth in SEQ ID NO: 58. In certain embodiments, the antibody comprises: (a) a light chain complementarity determining region 1 (LCDR1) comprising an amino acid sequence set forth in SEQ ID NO: 63; (b) a light chain complementarity determining region 2 (LCDR2) comprising an amino acid sequence set forth in SEQ ID NO: 64; (c) a light chain complementarity determining region 3 (LCDR3) comprising an amino acid sequence set forth in SEQ ID NO: 65; (d) a heavy chain complementarity determining region 1 (HCDR1) comprising an amino acid sequence set forth in SEQ ID NO: 66; (e) a heavy chain complementarity determining region 2 (HCDR2) comprising an amino acid sequence set forth in SEQ ID NO: 67; and/or (f) a heavy chain complementarity determining region 3 (HCDR3) comprising an amino acid sequence set forth in SEQ ID NO: 68. In certain embodiments, the antibody comprises: (a) a light chain complementarity determining region 1 (LCDR1) comprising an amino acid sequence set forth in SEQ ID NO: 73; (b) a light chain complementarity determining region 2 (LCDR2) comprising an amino acid sequence set forth in SEQ ID NO: 74; (c) a light chain complementarity determining region 3 (LCDR3) comprising an amino acid sequence set forth in SEQ ID NO: 75; (d) a heavy chain complementarity determining region 1 (HCDR1) comprising an amino acid sequence set forth in SEQ ID NO: 76; (e) a heavy chain complementarity determining region 2 (HCDR2) comprising an amino acid sequence set forth in SEQ ID NO: 77; and/or (f) a heavy chain complementarity determining region 3 (HCDR3) comprising an amino acid sequence set forth in SEQ ID NO: 78. In certain embodiments, the antibody comprises: (a) a light chain complementarity determining region 1 (LCDR1) comprising an amino acid sequence set forth in SEQ ID NO: 83; (b) a light chain complementarity determining region 2 (LCDR2) comprising an amino acid sequence set forth in SEQ ID NO: 84; (c) a light chain complementarity determining region 3 (LCDR3) comprising an amino acid sequence set forth in SEQ ID NO: 85; (d) a heavy chain complementarity determining region 1 (HCDR1) comprising an amino acid sequence set forth in SEQ ID NO: 86; (e) a heavy chain complementarity determining region 2 (HCDR2) comprising an amino acid sequence set forth in SEQ ID NO: 87; and/or (f) a heavy chain complementarity determining region 3 (HCDR3) comprising an amino acid sequence set forth in SEQ ID NO: 88. In certain embodiments, the antibody comprises an immunoglobulin light chain amino acid variable region sequence at least about 90%, 95%, 96%, 97%, 98%, or 99% identical to any one of SEQ ID NOs: 155, 157, 169, 267, 271, 275, 277, 309, 311, 313, 315, 317, or 319; and an immunoglobulin heavy chain variable region amino acid sequence at least about 90%, 95%, 96%, 97%, 98%, or 99% identical to any one of SEQ ID NOs: 156, 158, 170, 268, 272, 276, 278, 310, 312, 314, 316, 318, or 320. In certain embodiments, the antibody comprises an immunoglobulin light chain amino acid variable region sequence identical to any one of SEQ ID NOs: 155, 157, 169, 267, 271, 275, 277, 309, 311, 313, 315, 317, or 319; and an immunoglobulin heavy 1 chain variable region amino acid sequence identical to any one of SEQ ID NOs: 156, 158, 170, 268, 272, 276, 278, 310, 312, 314, 316, 318, or 320. In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to any one of the Kabat definition, Chothia definition, Aho definition, Abm definition, IMGT definition, Contact definition and North definition. In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined using a hybrid scheme of any two or three of the Kabat definition, Chothia definition, Aho definition, Abm definition, IMGT definition, Contact definition and North definition. In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to the Kabat definition. In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to the Chothia definition. In certain embodiments, the antibody comprises an immunoglobulin light chain variable region amino acid sequence at least about 90%, 95%, 96%, 97%, 98%, or 99% identical to SEQ ID NO: 275; and an immunoglobulin heavy chain variable region amino acid sequence at least about 90%, 95%, 96%, 97%, 98%, or 99% identical to SEQ ID NO: 276. In certain embodiments, the antibody comprises an immunoglobulin light chain variable region amino acid sequence identical to SEQ ID NOs: 275; and an immunoglobulin heavy chain variable region amino acid sequence identical to SEQ ID NO: 276. In certain embodiments, the antibody comprises an immunoglobulin light chain variable region amino acid sequence at least about 90%, 95%, 96%, 97%, 98%, or 99% identical to SEQ ID NO: 277; and an immunoglobulin heavy chain variable region amino acid sequence at least about 90%, 95%, 96%, 97%, 98%, or 99% identical to SEQ ID NO: 278. In certain embodiments, the antibody comprises an immunoglobulin light chain variable region amino acid sequence identical to SEQ ID NOs: 277; and an immunoglobulin heavy chain variable region amino acid sequence identical to SEQ ID NO: 278. In certain embodiments, the antibody comprises an immunoglobulin light chain variable region amino acid sequence at least about 90%, 95%, 96%, 97%, 98%, or 99% identical to SEQ ID NO: 309; and an immunoglobulin heavy chain variable region amino acid sequence at least about 90%, 95%, 96%, 97%, 98%, or 99% identical to SEQ ID NO: 310. In certain embodiments, the antibody comprises an immunoglobulin light chain variable region amino acid sequence identical to SEQ ID NOs: 309; and an immunoglobulin heavy chain variable region amino acid sequence identical to SEQ ID NO: 310. In certain embodiments, the antibody comprises an immunoglobulin light chain variable region amino acid sequence at least about 90%, 95%, 96%, 97%, 98%, or 99% identical to SEQ ID NO: 155; and an immunoglobulin heavy chain variable region amino acid sequence at least about 90%, 95%, 96%, 97%, 98%, or 99% identical to SEQ ID NO: 156. In certain embodiments, the antibody comprises an immunoglobulin light chain variable region amino acid sequence identical to SEQ ID NOs: 155; and an immunoglobulin heavy chain variable region amino acid sequence identical to SEQ ID NO: 156. In certain embodiments, the antibody comprises an immunoglobulin light chain variable region amino acid sequence at least about 90%, 95%, 96%, 97%, 98%, or 99% identical to SEQ ID NO: 157; and an immunoglobulin heavy chain variable region amino acid sequence at least about 90%, 95%, 96%, 97%, 98%, or 99% identical to SEQ ID NO: 158. In certain embodiments, the antibody comprises an immunoglobulin light chain variable region amino acid sequence identical to SEQ ID NOs: 157; and an immunoglobulin heavy chain variable region amino acid sequence identical to SEQ ID NO: 158. In certain embodiments, the antibody comprises an immunoglobulin light chain variable region amino acid sequence at least about 90%, 95%, 96%, 97%, 98%, or 99% identical to SEQ ID NO: 169; and an immunoglobulin heavy chain variable region amino acid sequence at least about 90%, 95%, 96%, 97%, 98%, or 99% identical to SEQ ID NO: 170. In certain embodiments, the antibody comprises an immunoglobulin light chain variable region amino acid sequence identical to SEQ ID NOs: 169; and an immunoglobulin heavy chain variable region amino acid sequence identical to SEQ ID NO: 170. In certain embodiments, the antibody comprises an immunoglobulin light chain variable region amino acid sequence at least about 90%, 95%, 96%, 97%, 98%, or 99% identical to SEQ ID NO: 267; and an immunoglobulin heavy chain variable region amino acid sequence at least about 90%, 95%, 96%, 97%, 98%, or 99% identical to SEQ ID NO: 268. In certain embodiments, the antibody comprises an immunoglobulin light chain variable region amino acid sequence identical to SEQ ID NOs: 267; and an immunoglobulin heavy chain variable region amino acid sequence identical to SEQ ID NO: 268. In certain embodiments, wherein the antibody comprises an immunoglobulin light chain variable region amino acid sequence at least about 90%, 95%, 96%, 97%, 98%, or 99% identical to SEQ ID NO: 271; and an immunoglobulin heavy chain variable region amino acid sequence at least about 90%, 95%, 96%, 97%, 98%, or 99% identical to SEQ ID NO: 272. In certain embodiments, the antibody comprises an immunoglobulin light chain variable region amino acid sequence identical to SEQ ID NOs: 271; and an immunoglobulin heavy chain variable region amino acid sequence identical to SEQ ID NO: 272. In certain embodiments, the antibody comprises an immunoglobulin light chain variable region amino acid sequence at least about 90%, 95%, 96%, 97%, 98%, or 99% identical to any one of the immunoglobulin light chain variable region sequences of Table 1; and an immunoglobulin heavy chain amino acid sequence at least about 90%, 95%, 96%, 97%, 98%, or 99% identical to any one of the immunoglobulin heavy chain variable region sequences of Table 1. In certain embodiments, the antibody comprises an immunoglobulin light chain amino acid sequence identical to any one of the immunoglobulin light chain variable region sequences of Table 1; and an immunoglobulin heavy chain variable region amino acid sequences identical to any one of the immunoglobulin heavy chain variable region sequences of Table 1. In certain embodiments, in the antibody comprises an arginine substitution at position number 17 of the immunoglobulin light chain according to Kabat numbering. In certain embodiments, wherein in the antibody comprises a tyrosine substitution at position number 12 of the immunoglobulin light chain according to Kabat numbering. In certain embodiments, described herein is a pharmaceutical composition comprising an antibody of this disclosure and a pharmaceutically acceptable carrier, diluent, or excipient. In certain embodiments, the antibody or pharmaceutical composition is formulated for intravenous administration. Also described is a nucleic acid encoding an antibody of this disclosure. In certain embodiments, described herein is an expression vector comprising a nucleic acid of this disclosure. Also described herein is a eukaryotic cell comprising the expression vector. In certain embodiments, the eukaryotic cell is a CHO cell line. Also described herein is a method of treating a cancer or tumor comprising administering to an individual afflicted with the cancer or tumor an antibody or a pharmaceutical composition comprising an antibody of the disclosure. In certain embodiments, the cancer or tumor is a solid tissue cancer. In certain embodiments, the cancer or tumor comprises breast cancer, colorectal cancer, or ovarian cancer. In certain embodiments, the breast cancer is triple negative breast cancer. In certain embodiments, the cancer or tumor is a blood cancer. Also described herein is a method of inhibiting CD24 signaling in a cancer cell comprising contacting the cancer cell to an antibody of the disclosure or a pharmaceutical composition of the disclosure. In certain embodiments, CD24 signaling is not inhibited in non-neoplastic B lymphocytes or non-neoplastic T lymphocytes. In certain embodiments, the cancer cell is a cancer cell is in vivo in an individual.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIGS. 1A and 1B illustrate dose response curves of maturated CD24 antibodies (single mutation) against CD24 (27-56aa)-GST (NEOX-4) by ELISA.

FIGS. 2A and 2B illustrate dose response curve of maturated CD24 antibodies (combined mutations) against CD24 (27-56aa)-GST (NEOX-4) by ELISA.

FIGS. 3A and 3B illustrate dose response curves of CD24 antibodies on NALM6 cells.

FIGS. 4A and 4B illustrate dose response curves of CD24 antibodies on MDA-MB-468 cells.

FIGS. 5A, 5B, and 5C illustrate the ADCC effect induced by anti-CD24 antibody on NALM6 and MDA-MB-468 cells.

FIG. 6A, 6B, 6C, and 6D illustrate the ADCP effect induced by anti-CD24 antibody on NALM6 and MDA-MB-468 cells.

FIGS. 7A, 7B, 7C and 7D illustrate dose response a binding curve with primary B cells and activated T cells.

FIG. 8 illustrates antibody binding by ELISA with N-Glycanase, Neuraminidase or N-Glycanase+Neuraminidase treated NEOX-4.

FIGS. 9A, 9B, and 9C illustrate CD24 antibody binding with N-Glycanase, Neuraminidase or N-Glycanase+Neuraminidase treated NALM6 (9A), MDA-MB-468 (9B), and primary B cells (9C).

FIG. 10 illustrates epitope binning by blocking peptides.

FIG. 11 illustrates epitope determination by alanine spanning mutagenesis.

FIG. 12 illustrates an epitope comparison from binning studies.

FIG. 13 illustrates CD24 antibody in vivo efficacy in the MC38-hCD24 syngeneic model.

FIGS. 14A and 14B illustrates CD24 antibody in vivo efficacy in the NALM6 CDX model.

FIGS. 15A and 15B illustrates CD24 antibody in vivo efficacy in the MDA-MB-468 CDX model.

### DETAILED DESCRIPTION

Described herein in one aspect is an antibody, or antigen binding fragment thereof, that binds to CD24, wherein the antibody does not bind to T lymphocytes. In certain embodiments, the antibody does not bind to activated T lymphocytes. In certain embodiments, the antibody does not bind to B lymphocytes.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

Among the provided antibodies are monoclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′) 2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG4 constant region. As used herein, the human constant region can comprise natural human immunoglobulin constant regions or variants thereof further comprising common modifications that confer altered effector functions, extended half-life or other properties. Such modifications include D356E, L358M, L234A, L235A, P329G, M252Y, S254T, and/or T256E per EU numbering.

The term “antibody” herein is used in the broadest sense and includes monoclonal antibodies, and includes intact antibodies and functional (antigen-binding) antibody fragments thereof, including fragment antigen binding (Fab) fragments, F(ab′) 2 fragments, Fab′ fragments, Fv fragments, recombinant IgG (rlgG) fragments, single chain antibody fragments (e.g., heavy chain antibodies), including single chain variable fragments (sFv or scFv), and single domain antibodies (e.g., sdAb, sdFv, nanobody, VHH) or fragments thereof. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

For preparation of suitable antibodies, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies of this disclosure. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

A “linker” herein is also referred to as “linker sequence” “spacer” “tethering sequence” or grammatical equivalents thereof. A “linker” as referred herein connects two distinct molecules that by themselves possess target binding, catalytic activity, or are naturally expressed and assembled as separate polypeptides, or comprise separate domains of the same polypeptide. For example, two distinct binding moieties or a heavy-chain/light-chain pair. A number of strategies may be used to covalently link molecules together. Linkers described herein may be utilized to join a light chain variable region and a heavy chain variable region in an scFv molecule; or may be used to tether an scFv or other antigen binding fragment on the N- or C-terminus of an antibody heavy chain; or the N- or C-terminus of a light chain to create a bispecific or multispecific binding molecule. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length may be used. Useful linkers include glycine-serine polymers, including for example (GS)n, (GSGGS)n, (GGGGS)n, and (GGGS)n, where n is an integer of at least one, glycine-alanine polymers, alanine-serine polymers, and other flexible linkers. Exemplary, linkers for linking antibody fragments or single chain variable fragments can include AAEPKSS, AAEPKSSDKTHTCPPCP, GGGG, or GGGGDKTHTCPPCP. Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers use as linkers.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

As described herein an “epitope” refers to the binding determinant of an antibody or fragment described herein minimally necessary for specific binding of the antibody or fragment thereof to a target antigen. When the target antigen is a polypeptide the epitope will be a continuous or discontinuous epitope. A continuous epitope is formed by one region of the target antigen, while a discontinuous epitope may be formed from two or more separate regions. A discontinuous epitope, for example, may form when a target antigen adopts a tertiary structure that brings two amino acid sequences together and forms a three-dimensional structure bound by the antibody. When the target antigen is a polypeptide the epitope will generally be a plurality of amino acids linked into a polypeptide chain. A continuous epitope may comprise 5, 6, 7, 8, 9, 10, 11, 12, 13, 14, 15, 16, 17, 18, 19, or 20 contiguous amino acids. While an epitope may comprise a contiguous polymer of amino acids, not every amino acid of the polymer may be contacted by an amino acid residue of the antibody. Such non-contacted amino acids will still comprise part of the epitope as they may be important for the structure and linkage of the contacted amino acids. The skilled artisan may determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

“CD24” refers to the sialoglycoprotein expressed at the surface of most B lymphocytes and activated T lymphocytes. The amino acid sequence of human CD24 can found at www.uniprot.org/uniprotkb/P25063/entry, with accession number P2506. In certain embodiments, the anti-CD24 binding antibodies described herein bind to mammalian CD24. In certain embodiments, the anti-CD24 binding antibodies described herein bind to human CD24.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn₂₉₇ of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn₂₉₇, relative to the sum of all glycostructures attached to Asn₂₉₇ (See e.g., WO 08/077546). Asn₂₉₇ refers to the asparagine residue located at about position 297 in the Fc region (EU numbering of Fc region residues; See e.g., Edelman et al. Proc Natl Acad Sci USA. 1969 May; 63 (1): 78-85). However, Asn₂₉₇ may also be located about ±3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87:614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87:614 (2004); Kanda, Y. et al., Biotechnol. Bioeng., 94 (4): 680-688 (2006)). Other antibody glycosylation variants are also included (See e.g., U.S. Pat. No. 6,602,684).

In some embodiments, an antibody provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the antibody target. In some embodiments, an antibody provided herein has a dissociation constant (K_{D}) of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or greater (e.g., 10⁻⁸ M or less, e.g., from 10⁻⁸ M to 10⁻¹³ M, e.g., from 10⁻⁹ M to 10⁻¹³ M) for the antibody target. The antibody target can be CD24. K_{D} can be measured by any suitable assay. In certain embodiments, K_{D} can be measured using surface plasmon resonance assays (e.g., using a BIACORE®-2000, a BIACORE®-3000 or Octet).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the Fc region of an immunoglobulin is important for many important antibody functions (e.g. effector functions), such as antigen-dependent cellular cytotoxicity (ADCC), complement dependent cytotoxicity (CDC), and antibody-dependent cell-mediated phagocytosis (ADCP), result in killing of target cells, albeit by different mechanisms. Accordingly, in some embodiments, the antibodies described herein comprise the variable domains of the invention combined with constant domains comprising different Fc regions, selected based on the biological activities of the antibody for the intended use. In certain instances, Human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32); FcγRIIIA and FcγRIIIB (CD16) and inhibiting receptor FcγRIIB), and for the first component of complement (Clq). Human IgG1 and IgG3 bind to all Fcγ receptors; IgG2 binds to FcγRIIAH131, and with lower affinity to FcγRIIAR131 FcγRIIIAv158; IgG4 binds to FcγRI, FcγRILA, FcγRIIB, FcγRIIC, and FcγRIIIAV158; and the inhibitory receptor FcγRIIB has a lower affinity for IgG1, IgG2 and IgG3 than all other Fcγ receptors. Studies have shown that FcγRI does not bind to IgG2, and FcγRIIIB does not bind to IgG2 or IgG4. Id. In general, with regard to ADCC activity, human IgG1≥lgG3>>IgG4≥IgG2.

In some embodiments, the antibodies of this disclosure are variants that possess reduced effector functions, which make it a desirable candidate for applications in which certain effector functions (such as complement fixation and ADCC) are unnecessary or deleterious. Such antibodies can have decreased complement-dependent cytotoxicity (CDC), antibody-dependent cell cytotoxicity (ADCC), or antibody dependent cellular phagocytosis (ADCP). In some embodiments, the antibodies of this disclosure are variants that possess increased effector functions for applications in which increased immunogenicity would be beneficial. Such antibodies can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone) polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large scale cell culture are known in the art. See e.g., Li et al., “Cell culture processes for monoclonal antibody production.” Mabs. 2010 September-October; 2 (5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NSO murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### CD24 Binding Antibodies

Described herein are antibodies that specifically bind to CD24. The antibodies described herein possess the useful properties of not binding to either B cells or activated T cells. Such antibodies may possess an enhanced safety profile with reduced immune side effects.

In certain embodiments, the antibody does not bind to T lymphocytes. In certain embodiments, the antibody has weak binding to T lymphocytes. In certain embodiments, antibody does not bind to activated T lymphocytes. In certain embodiments, the antibody has weak binding to activated T lymphocytes. In certain embodiments, said activated T lymphocytes are activated human T lymphocytes. In certain embodiments, the antibody does not bind to B lymphocytes. In certain embodiments, the antibody has weak binding to B lymphocytes. In certain embodiments, said B lymphocytes are human B lymphocytes. In certain embodiments, the antibody binds to CD24 with a KD of less than 1×10⁻⁸. In certain embodiments, the antibody binds to CD24 with a KD of less than 1×10⁻⁹. In certain embodiments, the antibody is a humanized or chimeric antibody. In certain embodiments, the antibody is a Fab, F (ab) 2, or a single chain variable fragment (scFv). In certain embodiments, the antibody is a heavy chain antibody or antigen binding fragment thereof. In certain embodiments, the antibody is an IgG antibody. In certain embodiments, the antibody is an IgG1 isotype. In certain embodiments, the antibody is an IgG4 isotype. In certain embodiments, the antibody binds to the serine at residue 18 of SEQ ID NO: 500. In certain embodiments, the antibody requires the presence of a serine at residue 18 of SEQ ID NO: 500 for binding. In certain embodiments, the antibody binds to the serine at residue 18 of SEQ ID NO: 500 but does not bind to the asparagine at residue 17 of SEQ ID NO: 500. In certain embodiments, the antibody binds to an epitope consisting of the amino acids sequence of SEQ ID NO 501. In certain embodiments, the antibody comprises: (a) a light chain complementarity determining region 1 (LCDR1) comprising an amino acid sequence set forth in SEQ ID NO: 401; (b) a light chain complementarity determining region 2 (LCDR2) comprising an amino acid sequence set forth in SEQ ID NO: 403; (c) a light chain complementarity determining region 3 (LCDR3) comprising an amino acid sequence set forth in SEQ ID NO: 405; (d) a heavy chain complementarity determining region 1 (HCDR1) comprising an amino acid sequence set forth in SEQ ID NO: 407; (e) a heavy chain complementarity determining region 2 (HCDR2) comprising an amino acid sequence set forth in SEQ ID NO: 409; and/or (f) a heavy chain complementarity determining region 3 (HCDR3) comprising an amino acid sequence set forth in SEQ ID NO: 411.

In certain embodiments, the antibody comprises: (a) a light chain complementarity determining region 1 (LCDR1) comprising an amino acid sequence set forth in SEQ ID NO: 402; (b) a light chain complementarity determining region 2 (LCDR2) comprising an amino acid sequence set forth in SEQ ID NO: 403; (c) a light chain complementarity determining region 3 (LCDR3) comprising an amino acid sequence set forth in SEQ ID NO: 406; (d) a heavy chain complementarity determining region 1 (HCDR1) comprising an amino acid sequence set forth in SEQ ID NO: 408; (e) a heavy chain complementarity determining region 2 (HCDR2) comprising an amino acid sequence set forth in SEQ ID NO: 409; and/or (f) a heavy chain complementarity determining region 3 (HCDR3) comprising an amino acid sequence set forth in SEQ ID NO: 412.

In certain embodiments, the antibody comprises: (a) a light chain complementarity determining region 1 (LCDR1) comprising an amino acid sequence selected from the group consisting of SEQ ID NO: 13, 23, 33, 43, 53, 63, 73, 83, 703, 713, 723, 733, and 743; (b) a light chain complementarity determining region 2 (LCDR2) comprising an amino acid sequence selected from the group consisting of SEQ ID NO: 14, 24, 34, 44, 54, 64, 74, 84, 704, 714, 724, 734, and 744; (c) a light chain complementarity determining region 3 (LCDR3) comprising having an amino acid sequence selected from the group consisting of SEQ ID NO: 15, 25, 35, 45, 55, 65, 75, 85, 705, 715, 725, 735, and 745; (d) a heavy chain complementarity determining region 1 (HCDR1) comprising an amino acid sequence selected from the group consisting of SEQ ID NO: 16, 26, 36, 46, 56, 66, 76, 86, 706, 716, 726, 736, and 746; (e) a heavy chain complementarity determining region 2 (HCDR2) comprising an amino acid sequence selected from the group consisting of SEQ ID NO: 17, 27, 37, 47, 57, 67, 77, 87, 707, 717, 727, 737, and 747; and/or (f) a heavy chain complementarity determining region 3 (HCDR3) comprising an amino acid sequence selected from the group consisting of SEQ ID NO: 18, 28, 38, 48, 58, 68, 78, 88, 708, 718, 728, 738, and 748.

In certain embodiments, the antibody comprises a combination of LCDRs 1-3 and HCDRs 1-3, wherein the combination is selected from the group consisting of: (i) LCDR1 comprising SEQ ID NO: 13, LCDR2 comprising SEQ ID NO: 14, LCDR3 comprising SEQ ID NO: 15, HCDR1 comprising SEQ ID NO: 16, HCDR2 comprising SEQ ID NO: 17, HCDR3 comprising SEQ ID NO: 18; (ii) LCDR1 comprising SEQ ID NO: 23, LCDR2 comprising SEQ ID NO: 24, LCDR3 comprising SEQ ID NO: 25, HCDR1 comprising SEQ ID NO: 26, HCDR2 comprising SEQ ID NO: 27, HCDR3 comprising SEQ ID NO: 28; (iii) LCDR1 comprising SEQ ID NO: 33, LCDR2 comprising SEQ ID NO: 34, LCDR3 comprising SEQ ID NO: 35, HCDR1 comprising SEQ ID NO: 36, HCDR2 comprising SEQ ID NO: 37, HCDR3 comprising SEQ ID NO: 38; (iv) LCDR1 comprising SEQ ID NO: 43, LCDR2 comprising SEQ ID NO: 44, LCDR3 comprising SEQ ID NO: 45, HCDR1 comprising SEQ ID NO: 46, HCDR2 comprising SEQ ID NO: 47, HCDR3 comprising SEQ ID NO: 48; (v) LCDR1 comprising SEQ ID NO: 53, LCDR2 comprising SEQ ID NO: 54, LCDR3 comprising SEQ ID NO: 55, HCDR1 comprising SEQ ID NO: 56, HCDR2 comprising SEQ ID NO: 57, HCDR3 comprising SEQ ID NO: 58; (vi) LCDR1 comprising SEQ ID NO: 63, LCDR2 comprising SEQ ID NO: 64, LCDR3 comprising SEQ ID NO: 65, HCDR1 comprising SEQ ID NO: 66, HCDR2 comprising SEQ ID NO: 67, HCDR3 comprising SEQ ID NO: 68; (vii) LCDR1 comprising SEQ ID NO: 73, LCDR2 comprising SEQ ID NO: 74, LCDR3 comprising SEQ ID NO: 75, HCDR1 comprising SEQ ID NO: 76, HCDR2 comprising SEQ ID NO: 77, HCDR3 comprising SEQ ID NO: 78; (viii) LCDR1 comprising SEQ ID NO: 83, LCDR2 comprising SEQ ID NO: 84, LCDR3 comprising SEQ ID NO: 85, HCDR1 comprising SEQ ID NO: 86, HCDR2 comprising SEQ ID NO: 87, HCDR3 comprising SEQ ID NO: 88; (ix) LCDR1 comprising SEQ ID NO: 703, LCDR2 comprising SEQ ID NO: 704, LCDR3 comprising SEQ ID NO: 705, HCDR1 comprising SEQ ID NO: 706, HCDR2 comprising SEQ ID NO: 707, HCDR3 comprising SEQ ID NO: 708; (x) LCDR1 comprising SEQ ID NO: 713, LCDR2 comprising SEQ ID NO: 714, LCDR3 comprising SEQ ID NO: 715, HCDR1 comprising SEQ ID NO: 716, HCDR2 comprising SEQ ID NO: 717, HCDR3 comprising SEQ ID NO: 718; (xi) LCDR1 comprising SEQ ID NO: 723, LCDR2 comprising SEQ ID NO: 724, LCDR3 comprising SEQ ID NO: 725, HCDR1 comprising SEQ ID NO: 726, HCDR2 comprising SEQ ID NO: 727, HCDR3 comprising SEQ ID NO: 728; (xii) LCDR1 comprising SEQ ID NO: 733, LCDR2 comprising SEQ ID NO: 734, LCDR3 comprising SEQ ID NO: 735, HCDR1 comprising SEQ ID NO: 736, HCDR2 comprising SEQ ID NO: 737, HCDR3 comprising SEQ ID NO: 738; and (xiii) LCDR1 comprising SEQ ID NO: 743, LCDR2 comprising SEQ ID NO: 744, LCDR3 comprising SEQ ID NO: 745, HCDR1 comprising SEQ ID NO: 746, HCDR2 comprising SEQ ID NO: 747, HCDR3 comprising SEQ ID NO: 748.

In certain embodiments, the antibody comprises a combination of LCDRs 1-3 and HCDRs 1-3, wherein the combination is selected from the group consisting of: (i) LCDR1 as set forth in SEQ ID NO: 13, LCDR2 as set forth in SEQ ID NO: 14, LCDR3 as set forth in SEQ ID NO: 15, HCDR1 as set forth in SEQ ID NO: 16, HCDR2 as set forth in SEQ ID NO: 17, HCDR3 as set forth in SEQ ID NO: 18; (ii) LCDR1 as set forth in SEQ ID NO: 23, LCDR2 as set forth in SEQ ID NO: 24, LCDR3 as set forth in SEQ ID NO: 25, HCDR1 as set forth in SEQ ID NO: 26, HCDR2 as set forth in SEQ ID NO: 27, HCDR3 as set forth in SEQ ID NO: 28; (iii) LCDR1 as set forth in SEQ ID NO: 33, LCDR2 as set forth in SEQ ID NO: 34, LCDR3 as set forth in SEQ ID NO: 35, HCDR1 as set forth in SEQ ID NO: 36, HCDR2 as set forth in SEQ ID NO: 37, HCDR3 as set forth in SEQ ID NO: 38; (iv) LCDR1 as set forth in SEQ ID NO: 43, LCDR2 as set forth in SEQ ID NO: 44, LCDR3 as set forth in SEQ ID NO: 45, HCDR1 as set forth in SEQ ID NO: 46, HCDR2 as set forth in SEQ ID NO: 47, HCDR3 as set forth in SEQ ID NO: 48; (v) LCDR1 as set forth in SEQ ID NO: 53, LCDR2 as set forth in SEQ ID NO: 54, LCDR3 as set forth in SEQ ID NO: 55, HCDR1 as set forth in SEQ ID NO: 56, HCDR2 as set forth in SEQ ID NO: 57, HCDR3 as set forth in SEQ ID NO: 58; (vi) LCDR1 as set forth in SEQ ID NO: 63, LCDR2 as set forth in SEQ ID NO: 64, LCDR3 as set forth in SEQ ID NO: 65, HCDR1 as set forth in SEQ ID NO: 66, HCDR2 as set forth in SEQ ID NO: 67, HCDR3 as set forth in SEQ ID NO: 68; (vii) LCDR1 as set forth in SEQ ID NO: 73, LCDR2 as set forth in SEQ ID NO: 74, LCDR3 as set forth in SEQ ID NO: 75, HCDR1 as set forth in SEQ ID NO: 76, HCDR2 as set forth in SEQ ID NO: 77, HCDR3 as set forth in SEQ ID NO: 78; (viii) LCDR1 as set forth in SEQ ID NO: 83, LCDR2 as set forth in SEQ ID NO: 84, LCDR3 as set forth in SEQ ID NO: 85, HCDR1 as set forth in SEQ ID NO: 86, HCDR2 as set forth in SEQ ID NO: 87, HCDR3 as set forth in SEQ ID NO: 88; (ix) LCDR1 as set forth in SEQ ID NO: 703, LCDR2 as set forth in SEQ ID NO: 704, LCDR3 as set forth in SEQ ID NO: 705, HCDR1 as set forth in SEQ ID NO: 706, HCDR2 as set forth in SEQ ID NO: 707, HCDR3 as set forth in SEQ ID NO: 708; (x) LCDR1 as set forth in SEQ ID NO: 713, LCDR2 as set forth in SEQ ID NO: 714, LCDR3 as set forth in SEQ ID NO: 715, HCDR1 as set forth in SEQ ID NO: 716, HCDR2 as set forth in SEQ ID NO: 717, HCDR3 as set forth in SEQ ID NO: 718; (xi) LCDR1 as set forth in SEQ ID NO: 723, LCDR2 as set forth in SEQ ID NO: 724, LCDR3 as set forth in SEQ ID NO: 725, HCDR1 as set forth in SEQ ID NO: 726, HCDR2 as set forth in SEQ ID NO: 727, HCDR3 as set forth in SEQ ID NO: 728; (xii) LCDR1 as set forth in SEQ ID NO: 733, LCDR2 as set forth in SEQ ID NO: 734, LCDR3 as set forth in SEQ ID NO: 735, HCDR1 as set forth in SEQ ID NO: 736, HCDR2 as set forth in SEQ ID NO: 737, HCDR3 as set forth in SEQ ID NO: 738; and (xiii) LCDR1 as set forth in SEQ ID NO: 743, LCDR2 as set forth in SEQ ID NO: 744, LCDR3 as set forth in SEQ ID NO: 745, HCDR1 as set forth in SEQ ID NO: 746, HCDR2 as set forth in SEQ ID NO: 747, HCDR3 as set forth in SEQ ID NO: 748.

In certain embodiments, the antibody comprises: (a) a light chain complementarity determining region 1 (LCDR1) comprising an amino acid sequence set forth in SEQ ID NO: 13; (b) a light chain complementarity determining region 2 (LCDR2) comprising an amino acid sequence set forth in SEQ ID NO: 14; (c) a light chain complementarity determining region 3 (LCDR3) comprising an amino acid sequence set forth in SEQ ID NO: 15; (d) a heavy chain complementarity determining region 1 (HCDR1) comprising an amino acid sequence set forth in SEQ ID NO: 16; (e) a heavy chain complementarity determining region 2 (HCDR2) comprising an amino acid sequence set forth in SEQ ID NO: 17; and/or (f) a heavy chain complementarity determining region 3 (HCDR3) comprising an amino acid sequence set forth in SEQ ID NO: 18.

In certain embodiments, the antibody comprises: (a) a light chain complementarity determining region 1 (LCDR1) comprising an amino acid sequence set forth in SEQ ID NO: 23; (b) a light chain complementarity determining region 2 (LCDR2) comprising an amino acid sequence set forth in SEQ ID NO: 24; (c) a light chain complementarity determining region 3 (LCDR3) comprising an amino acid sequence set forth in SEQ ID NO: 25; (d) a heavy chain complementarity determining region 1 (HCDR1) comprising an amino acid sequence set forth in SEQ ID NO: 26; (e) a heavy chain complementarity determining region 2 (HCDR2) comprising an amino acid sequence set forth in SEQ ID NO: 27; and/or (f) a heavy chain complementarity determining region 3 (HCDR3) comprising an amino acid sequence set forth in SEQ ID NO: 28. In certain embodiments, the antibody comprises: (a) a light chain complementarity determining region 1 (LCDR1) comprising an amino acid sequence set forth in SEQ ID NO: 33; (b) a light chain complementarity determining region 2 (LCDR2) comprising an amino acid sequence set forth in SEQ ID NO: 34; (c) a light chain complementarity determining region 3 (LCDR3) comprising an amino acid sequence set forth in SEQ ID NO: 35; (d) a heavy chain complementarity determining region 1 (HCDR1) comprising an amino acid sequence set forth in SEQ ID NO: 36; € a heavy chain complementarity determining region 2 (HCDR2) comprising an amino acid sequence set forth in SEQ ID NO: 37; and/or (f) a heavy chain complementarity determining region 3 (HCDR3) comprising an amino acid sequence set forth in SEQ ID NO: 38.

In certain embodiments, the antibody comprises: (a) a light chain complementarity determining region 1 (LCDR1) comprising an amino acid sequence set forth in SEQ ID NO: 43; (b) a light chain complementarity determining region 2 (LCDR2) comprising an amino acid sequence set forth in SEQ ID NO: 44; (c) a light chain complementarity determining region 3 (LCDR3) comprising an amino acid sequence set forth in SEQ ID NO: 45; (d) a heavy chain complementarity determining region 1 (HCDR1) comprising an amino acid sequence set forth in SEQ ID NO: 46; (e) a heavy chain complementarity determining region 2 (HCDR2) comprising an amino acid sequence set forth in SEQ ID NO: 47; and/or (f) a heavy chain complementarity determining region 3 (HCDR3) comprising an amino acid sequence set forth in SEQ ID NO: 48.

In certain embodiments, the antibody comprises: (a) a light chain complementarity determining region 1 (LCDR1) comprising an amino acid sequence set forth in SEQ ID NO: 53; (b) a light chain complementarity determining region 2 (LCDR2) comprising an amino acid sequence set forth in SEQ ID NO: 54; (c) a light chain complementarity determining region 3 (LCDR3) comprising an amino acid sequence set forth in SEQ ID NO: 55; (d) a heavy chain complementarity determining region 1 (HCDR1) comprising an amino acid sequence set forth in SEQ ID NO: 56; (e) a heavy chain complementarity determining region 2 (HCDR2) comprising an amino acid sequence set forth in SEQ ID NO: 57; and/or (f) a heavy chain complementarity determining region 3 (HCDR3) comprising an amino acid sequence set forth in SEQ ID NO: 58.

In certain embodiments, the antibody comprises: (a) a light chain complementarity determining region 1 (LCDR1) comprising an amino acid sequence set forth in SEQ ID NO: 63; (b) a light chain complementarity determining region 2 (LCDR2) comprising an amino acid sequence set forth in SEQ ID NO: 64; (c) a light chain complementarity determining region 3 (LCDR3) comprising an amino acid sequence set forth in SEQ ID NO: 65; (d) a heavy chain complementarity determining region 1 (HCDR1) comprising an amino acid sequence set forth in SEQ ID NO: 66; (e) a heavy chain complementarity determining region 2 (HCDR2) comprising an amino acid sequence set forth in SEQ ID NO: 67; and/or (f) a heavy chain complementarity determining region 3 (HCDR3) comprising an amino acid sequence set forth in SEQ ID NO: 68.

In certain embodiments, the antibody comprises: (a) a light chain complementarity determining region 1 (LCDR1) comprising an amino acid sequence set forth in SEQ ID NO: 73; (b) a light chain complementarity determining region 2 (LCDR2) comprising an amino acid sequence set forth in SEQ ID NO: 74; (c) a light chain complementarity determining region 3 (LCDR3) comprising an amino acid sequence set forth in SEQ ID NO: 75; (d) a heavy chain complementarity determining region 1 (HCDR1) comprising an amino acid sequence set forth in SEQ ID NO: 76; (e) a heavy chain complementarity determining region 2 (HCDR2) comprising an amino acid sequence set forth in SEQ ID NO: 77; and/or (f) a heavy chain complementarity determining region 3 (HCDR3) comprising an amino acid sequence set forth in SEQ ID NO: 78.

In certain embodiments, the antibody comprises: (a) a light chain complementarity determining region 1 (LCDR1) comprising an amino acid sequence set forth in SEQ ID NO: 83; (b) a light chain complementarity determining region 2 (LCDR2) comprising an amino acid sequence set forth in SEQ ID NO: 84; (c) a light chain complementarity determining region 3 (LCDR3) comprising an amino acid sequence set forth in SEQ ID NO: 85; (d) a heavy chain complementarity determining region 1 (HCDR1) comprising an amino acid sequence set forth in SEQ ID NO: 86; (e) a heavy chain complementarity determining region 2 (HCDR2) comprising an amino acid sequence set forth in SEQ ID NO: 87; and/or (f) a heavy chain complementarity determining region 3 (HCDR3) comprising an amino acid sequence set forth in SEQ ID NO: 88.

In certain embodiments, the antibody comprises an immunoglobulin light chain amino acid variable region sequence at least about 90%, 95%, 96%, 97%, 98%, or 99% identical to any one of SEQ ID NOs: 155, 157, 169, 267, 271, 275, 277, 309, 311, 313, 315, 317, or 319; and an immunoglobulin heavy chain variable region amino acid sequence at least about 90%, 95%, 96%, 97%, 98%, or 99% identical to any one of SEQ ID NOs: 156, 158, 170, 268, 272, 276, 278, 310, 312, 314, 316, 318, or 320.

In certain embodiments, the antibody comprises an immunoglobulin light chain amino acid variable region sequence identical to any one of SEQ ID NOs: 155, 157, 169, 267, 271, 275, 277, 309, 311, 313, 315, 317, or 319; and an immunoglobulin heavy 1 chain variable region amino acid sequence identical to any one of SEQ ID NOs: 156, 158, 170, 268, 272, 276, 278, 310, 312, 314, 316, 318, or 320.

In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to any one of the Kabat definition, Chothia definition, Aho definition, Abm definition, IMGT definition, Contact definition and North definition.

In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined using a hybrid scheme of any two or three of the Kabat definition, Chothia definition, Aho definition, Abm definition, IMGT definition, Contact definition and North definition.

In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to the Kabat definition.

In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to the Chothia definition.

In certain embodiments, the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to Kabat combined with Chothia definitions.

In certain embodiments, the antibody comprises an immunoglobulin light chain variable region amino acid sequence at least about 90%, 95%, 96%, 97%, 98%, or 99% identical to SEQ ID NO: 275; and an immunoglobulin heavy chain variable region amino acid sequence at least about 90%, 95%, 96%, 97%, 98%, or 99% identical to SEQ ID NO: 276.

In certain embodiments, the antibody comprises an immunoglobulin light chain variable region amino acid sequence identical to SEQ ID NOs: 275; and an immunoglobulin heavy chain variable region amino acid sequence identical to SEQ ID NO: 276.

In certain embodiments, the antibody comprises an immunoglobulin light chain variable region amino acid sequence at least about 90%, 95%, 96%, 97%, 98%, or 99% identical to SEQ ID NO: 277; and an immunoglobulin heavy chain variable region amino acid sequence at least about 90%, 95%, 96%, 97%, 98%, or 99% identical to SEQ ID NO: 278.

In certain embodiments, the antibody comprises an immunoglobulin light chain variable region amino acid sequence identical to SEQ ID NOs: 277; and an immunoglobulin heavy chain variable region amino acid sequence identical to SEQ ID NO: 278.

In certain embodiments, the antibody comprises an immunoglobulin light chain variable region amino acid sequence at least about 90%, 95%, 96%, 97%, 98%, or 99% identical to SEQ ID NO: 309; and an immunoglobulin heavy chain variable region amino acid sequence at least about 90%, 95%, 96%, 97%, 98%, or 99% identical to SEQ ID NO: 310.

In certain embodiments, the antibody comprises an immunoglobulin light chain variable region amino acid sequence identical to SEQ ID NOs: 309; and an immunoglobulin heavy chain variable region amino acid sequence identical to SEQ ID NO: 310.

In certain embodiments, the antibody comprises an immunoglobulin light chain variable region amino acid sequence at least about 90%, 95%, 96%, 97%, 98%, or 99% identical to SEQ ID NO: 155; and an immunoglobulin heavy chain variable region amino acid sequence at least about 90%, 95%, 96%, 97%, 98%, or 99% identical to SEQ ID NO: 156.

In certain embodiments, the antibody comprises an immunoglobulin light chain variable region amino acid sequence identical to SEQ ID NOs: 155; and an immunoglobulin heavy chain variable region amino acid sequence identical to SEQ ID NO: 156.

In certain embodiments, the antibody comprises an immunoglobulin light chain variable region amino acid sequence at least about 90%, 95%, 96%, 97%, 98%, or 99% identical to SEQ ID NO: 157; and an immunoglobulin heavy chain variable region amino acid sequence at least about 90%, 95%, 96%, 97%, 98%, or 99% identical to SEQ ID NO: 158.

In certain embodiments, the antibody comprises an immunoglobulin light chain variable region amino acid sequence identical to SEQ ID NOs: 157; and an immunoglobulin heavy chain variable region amino acid sequence identical to SEQ ID NO: 158.

In certain embodiments, the antibody comprises an immunoglobulin light chain variable region amino acid sequence at least about 90%, 95%, 96%, 97%, 98%, or 99% identical to SEQ ID NO: 169; and an immunoglobulin heavy chain variable region amino acid sequence at least about 90%, 95%, 96%, 97%, 98%, or 99% identical to SEQ ID NO: 170.

In certain embodiments, the antibody comprises an immunoglobulin light chain variable region amino acid sequence identical to SEQ ID NOs: 169; and an immunoglobulin heavy chain variable region amino acid sequence identical to SEQ ID NO: 170.

In certain embodiments, the antibody comprises an immunoglobulin light chain variable region amino acid sequence at least about 90%, 95%, 96%, 97%, 98%, or 99% identical to SEQ ID NO: 267; and an immunoglobulin heavy chain variable region amino acid sequence at least about 90%, 95%, 96%, 97%, 98%, or 99% identical to SEQ ID NO: 268.

In certain embodiments, the antibody comprises an immunoglobulin light chain variable region amino acid sequence identical to SEQ ID NOs: 267; and an immunoglobulin heavy chain variable region amino acid sequence identical to SEQ ID NO: 268.

In certain embodiments, the antibody comprises an immunoglobulin light chain variable region amino acid sequence at least about 90%, 95%, 96%, 97%, 98%, or 99% identical to SEQ ID NO: 271; and an immunoglobulin heavy chain variable region amino acid sequence at least about 90%, 95%, 96%, 97%, 98%, or 99% identical to SEQ ID NO: 272.

In certain embodiments, the antibody comprises an immunoglobulin light chain variable region amino acid sequence identical to SEQ ID NOs: 271; and an immunoglobulin heavy chain variable region amino acid sequence identical to SEQ ID NO: 272.

In certain embodiments, the antibody comprises an immunoglobulin light chain variable region amino acid sequence at least about 90%, 95%, 96%, 97%, 98%, or 99% identical to a immunoglobulin light chain variable region sequence of Table 1; and an immunoglobulin heavy chain amino acid sequence at least about 90%, 95%, 96%, 97%, 98%, or 99% identical to a heavy chain immunoglobulin sequence of Table 1.

In certain embodiments, the antibody comprises an arginine substitution at position number 17 of the immunoglobulin light chain according to Kabat numbering.

In certain embodiments, the antibody comprises a tyrosine substitution at position number 12 of the immunoglobulin light chain according to Kabat numbering.

In certain embodiments the antibody comprises an immunoglobulin light chain variable region amino acid sequence at least about 90%, 95%, 96%, 97%, 98%, or 99% or 100% identical to any one of the immunoglobulin light chain variable region sequences of Table 1; and an immunoglobulin heavy chain amino acid sequence at least about 90%, 95%, 96%, 97%, 98%, or 99% or 100% identical to any one of the immunoglobulin heavy chain variable region sequences of Table 1.

In certain embodiments, the antibody comprises an immunoglobulin light chain amino acid sequence identical to any one of the immunoglobulin light chain variable region sequences of Table 1; and an immunoglobulin heavy chain variable region amino acid sequence identical to any one of the immunoglobulin heavy chain variable region sequences of Table 1.

TABLE 1

| SEQ ID | SEQUENCE | | clone |
|---|---|---|---|
| 1 | DVVMTQTPLTLSVTIGQPASISCKSSQSLLYSNGETYLN | VL | G14 |
| | WLLQRPGQSPKRLIYLVSKLDSGVPDRVTGSGSGTDFT | | |
| | LKISRVEAEDLGVYYCVQGTYFPYTFGGGTKLEIK | | |
| 2 | EVKLEESGGGLVQPGGSMKLSCVASGLTFSNYWMNW | VH | G14 |
| | VRQSPEKGLEWVALIRLYSDNYAIHYAESVKGRFTISR | | |
| | DDSKSSVYLQMNNLRAEDTGIYYCTTGTDYWGQGTT | | |
| | LTVSS | | |
| 3 | KSSQSLLYSNGETYLN | LCDR1 | G14 |
| 4 | LVSKLDS | LCDR2 | G14 |
| 5 | VQGTYFPYT | LCDR3 | G14 |
| 6 | NYWMN | HCDR1 | G14 |
| 7 | LIRLYSDNYAIHYAESVKG | HCDR2 | G14 |
| 8 | GTDY | HCDR3 | G14 |
| 13 | KSSHSLLYSNKKTYLN | LCDR1 | huG14-234 |
| 14 | LVSKLDS | LCDR2 | huG14-234 |
| 15 | IQGTYFPYT | LCDR3 | huG14-234 |
| 16 | NYWGN | HCDR1 | huG14-234 |
| 17 | LIRLYSDNYAIHYAESVKG | HCDR2 | huG14-234 |
| 18 | GTDY | HCDR3 | huG14-234 |
| 23 | KSSHSLLYSNKRTYLN | LCDR1 | huG14-235 |
| 24 | LVSKLDS | LCDR2 | huG14-235 |
| 25 | IQGTYFPYT | LCDR3 | huG14-235 |
| 26 | NYWGN | HCDR1 | huG14-235 |
| 27 | LIRLYSDNYAIHYAESVKG | HCDR2 | huG14-235 |
| 28 | GTDN | HCDR3 | huG14-235 |
| 33 | KSSHSLLYSNKRTYLN | LCDR1 | huG14-301 |
| 34 | LVSKLDS | LCDR2 | huG14-301 |
| 35 | IQGTYFPYT | LCDR3 | huG14-301 |
| 36 | NYWGN | HCDR1 | huG14-301 |
| 37 | LIRLYSDNYAIHYAESVKG | HCDR2 | huG14-301 |
| 38 | GTDN | HCDR3 | huG14-301 |
| 43 | KSSHSLLYSNGKTYLN | LCDR1 | huG14-124 |
| 44 | LVSKLDS | LCDR2 | huG14-124 |
| 45 | IQGTYFPYT | LCDR3 | huG14-124 |
| 46 | NYWGN | HCDR1 | huG14-124 |
| 47 | LIRLYSDNYAIHYAESVKG | HCDR2 | huG14-124 |
| 48 | GTDY | HCDR3 | huG14-124 |
| 53 | KSSHSLLYSNGRTYLN | LCDR1 | huG14-125 |
| 54 | LVSKLDS | LCDR2 | huG14-125 |
| 55 | IQGTYFPYT | LCDR3 | huG14-125 |
| 56 | NYWGN | HCDR1 | huG14-125 |
| 57 | LIRLYSDNYAIHYAESVKG | HCDR2 | huG14-125 |
| 58 | GTDY | HCDR3 | huG14-125 |
| 63 | KSSHSLLYSNGKTYLN | LCDR1 | huG14-131 |
| 64 | LVSKLDS | LCDR2 | huG14-131 |
| 65 | IQGTYFPYT | LCDR3 | huG14-131 |
| 66 | NYWMN | HCDR1 | huG14-131 |
| 67 | LIRLYSDNYAIHYAESVKG | HCDR2 | huG14-131 |
| 68 | GTDN | HCDR3 | huG14-131 |
| 73 | KSSHSLLYSNGKTYLN | LCDR1 | huG14-230 |
| 74 | LVSKLDS | LCDR2 | huG14-230 |
| 75 | IQGTYFPYT | LCDR3 | huG14-230 |
| 76 | NYWGN | HCDR1 | huG14-230 |
| 77 | LIRLYSDNYAIHYAESVKG | HCDR2 | huG14-230 |
| 78 | GTDN | HCDR3 | huG14-230 |
| 83 | KSSHSLLYSNGRTYLN | LCDR1 | huG14-232 |
| 84 | LVSKLDS | LCDR2 | huG14-232 |
| 85 | IQGTYFPYT | LCDR3 | huG14-232 |
| 86 | NYWGN | HCDR1 | huG14-232 |
| 87 | LIRLYSDNYAIHYAESVKG | HCDR2 | huG14-232 |
| 88 | GTDN | HCDR3 | huG14-232 |
| 101 | DVVMTQTPLTLSVTIGQPASISCKSSQSLLYSNGETYLN | VL | huG14-1 |
| | WLLQRPGQSPKRLIYLVSKLDSGVPDRVTGSGSGTDFT | | |
| | LKISRVEAEDLGVYYCVQGTYFPYTFGGGTKLEIK | | |
| 102 | EVQLVESGGGLVKPGGSLRLSCAASGLTFSNYWMNW | VH | huG14-1 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 103 | KSSQSLLYSNGETYLN | LCDR1 | huG14-1 |
| 104 | LVSKLDS | LCDR2 | huG14-1 |
| 105 | VQGTYFPYT | LCDR3 | huG14-1 |
| 106 | SNYWMN | HCDR1 | huG14-1 |
| 107 | LIRLYSDNYAIHYAESVKG | HCDR2 | huG14-1 |
| 108 | GTDY | HCDR3 | huG14-1 |
| 109 | DVVMTQTPLTLSVTIGQPASISCKSSFSLLYSNGETYLN | VL | huG14-101 |
| | WLLQRPGQSPKRLIYLVSKLDSGVPDRVTGSGSGTDFT | | |
| | LKISRVEAEDLGVYYCVQGTYFPYTFGGGTKLEIK | | |
| 110 | EVQLVESGGGLVKPGGSLRLSCAASGLTFSNYWMNW | VH | huG14-101 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 111 | DVVMTQTPLTLSVTIGQPASISCKSSHSLLYSNGETYLN | VL | huG14-102 |
| | WLLQRPGQSPKRLIYLVSKLDSGVPDRVTGSGSGTDFT | | |
| | LKISRVEAEDLGVYYCVQGTYFPYTFGGGTKLEIK | | |
| 112 | EVQLVESGGGLVKPGGSLRLSCAASGLTFSNYWMNW | VH | huG14-102 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 113 | DVVMTQTPLTLSVTIGQPASISCKSSQSLLYSNGETYLN | VL | huG14-103 |
| | WLLQRPGQSPKRLIYLVSKLDSGVPDRVTGSGSGTDFT | | |
| | LKISRVEAEDLGVYYCIQGTYFPYTFGGGTKLEIK | | |
| 114 | EVQLVESGGGLVKPGGSLRLSCAASGLTFSNYWMNW | VH | huG14-103 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 115 | DVVMTQTPLTLSVTIGQPASISCKSSQSLLYSNGITYLN | VL | huG14-104 |
| | WLLQRPGQSPKRLIYLVSKLDSGVPDRVTGSGSGTDFT | | |
| | LKISRVEAEDLGVYYCVQGTYFPYTFGGGTKLEIK | | |
| 116 | EVQLVESGGGLVKPGGSLRLSCAASGLTFSNYWMNW | VH | huG14-104 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 117 | DVVMTQTPLTLSVTIGQPASISCKSSQSLLYSNGKTYLN | VL | huG14-105 |
| | WLLQRPGQSPKRLIYLVSKLDSGVPDRVTGSGSGTDFT | | |
| | LKISRVEAEDLGVYYCVQGTYFPYTFGGGTKLEIK | | |
| 118 | EVQLVESGGGLVKPGGSLRLSCAASGLTFSNYWMNW | VH | huG14-105 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 119 | DVVMTQTPLTLSVTIGQPASISCKSSQSLLYSNGRTYLN | VL | huG14-106 |
| | WLLQRPGQSPKRLIYLVSKLDSGVPDRVTGSGSGTDFT | | |
| | LKISRVEAEDLGVYYCVQGTYFPYTFGGGTKLEIK | | |
| 120 | EVQLVESGGGLVKPGGSLRLSCAASGLTFSNYWMNW | VH | huG14-106 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 121 | DVVMTQTPLTLSVTIGQPASISCKSSQSLLYSNGVTYLN | VL | huG14-107 |
| | WLLQRPGQSPKRLIYLVSKLDSGVPDRVTGSGSGTDFT | | |
| | LKISRVEAEDLGVYYCVQGTYFPYTFGGGTKLEIK | | |
| 122 | EVQLVESGGGLVKPGGSLRLSCAASGLTFSNYWMNW | VH | huG14-107 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 123 | DVVMTQTPLTLSVTIGQPASISCKSSQSLLYSNWETYL | VL | huG14-108 |
| | NWLLQRPGQSPKRLIYLVSKLDSGVPDRVTGSGSGTDF | | |
| | TLKISRVEAEDLGVYYCVQGTYFPYTFGGGTKLEIK | | |
| 124 | EVQLVESGGGLVKPGGSLRLSCAASGLTFSNYWMNW | VH | huG14-108 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 125 | DVVMTQTPLTLSVTIGQPASISCKSSQSLLYSNGETYLN | VL | huG14-109 |
| | WLLQRPGQSPKRLIYLVSKLDSGVPDRVTGSGSGTDFT | | |
| | LKISRVEAEDLGVYYCVQGTYFPYTFGGGTKLEIK | | |
| 126 | EVQLVESGGGLVKPGGSLRLSCAASGLTFSNYWGNW | VH | huG14-109 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 127 | DVVMTQTPLTLSVTIGQPASISCKSSQSLLYSNGETYLN | VL | huG14-110 |
| | WLLQRPGQSPKRLIYLVSKLDSGVPDRVTGSGSGTDFT | | |
| | LKISRVEAEDLGVYYCVQGTYFPYTFGGGTKLEIK | | |
| 128 | EVQLVESGGGLVKPGGSLRLSCAASGLTFSNYWMNW | VH | huG14-110 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 129 | DVVMTQTPLTLSVTIGQPASISCKSSQSLLYSNGETYLN | VL | huG14-111 |
| | WLLQRPGQSPKRLIYLVSKLDSGVPDRVTGSGSGTDFT | | |
| | LKISRVEAEDLGVYYCVQGTYFPYTFGGGTKLEIK | | |
| 130 | EVQLVESGGGLVKPGGSLRLSCAASGLTFSNYWMNW | VH | huG14-111 |
| | VRQAPGKGLEWVALIRLYSDNYAIHYAESVKGRFTISR | | |
| | DDSKSTVYLQMNSLKTEDTAVYYCTTGTDFWGQGTT | | |
| | VTVSS | | |
| 131 | DVVMTQTPLTLSVTIGQPASISCKSSHSLLYSNGETYLN | VL | huG14-112 |
| | WLLQRPGQSPKRLIYLVSKLDSGVPDRVTGSGSGTDFT | | |
| | LKISRVEAEDLGVYYCIQGTYFPYTFGGGTKLEIK | | |
| 132 | EVQLVESGGGLVKPGGSLRLSCAASGLTFSNYWMNW | VH | huG14-112 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 133 | DVVMTQTPLTLSVTIGQPASISCKSSHSLLYSNGKTYLN | VL | huG14-113 |
| | WLLQRPGQSPKRLIYLVSKLDSGVPDRVTGSGSGTDFT | | |
| | LKISRVEAEDLGVYYCVQGTYFPYTFGGGTKLEIK | | |
| 134 | EVQLVESGGGLVKPGGSLRLSCAASGLTFSNYWMNW | VH | huG14-113 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 135 | DVVMTQTPLTLSVTIGQPASISCKSSHSLLYSNGRTYLN | VL | huG14-114 |
| | WLLQRPGQSPKRLIYLVSKLDSGVPDRVTGSGSGTDFT | | |
| | LKISRVEAEDLGVYYCVQGTYFPYTFGGGTKLEIK | | |
| 136 | EVQLVESGGGLVKPGGSLRLSCAASGLTFSNYWMNW | VH | huG14-114 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 137 | DVVMTQTPLTLSVTIGQPASISCKSSQSLLYSNGKTYLN | VL | huG14-115 |
| | WLLQRPGQSPKRLIYLVSKLDSGVPDRVTGSGSGTDFT | | |
| | LKISRVEAEDLGVYYCIQGTYFPYTFGGGTKLEIK | | |
| 138 | EVQLVESGGGLVKPGGSLRLSCAASGLTFSNYWMNW | VH | huG14-115 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 139 | DVVMTQTPLTLSVTIGQPASISCKSSQSLLYSNGRTYLN | VL | huG14-116 |
| | WLLQRPGQSPKRLIYLVSKLDSGVPDRVTGSGSGTDFT | | |
| | LKISRVEAEDLGVYYCIQGTYFPYTFGGGTKLEIK | | |
| 140 | EVQLVESGGGLVKPGGSLRLSCAASGLTFSNYWMNW | VH | huG14-116 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 141 | DVVMTQTPLTLSVTIGQPASISCKSSHSLLYSNGKTYLN | VL | huG14-117 |
| | WLLQRPGQSPKRLIYLVSKLDSGVPDRVTGSGSGTDFT | | |
| | LKISRVEAEDLGVYYCIQGTYFPYTFGGGTKLEIK | | |
| 142 | EVQLVESGGGLVKPGGSLRLSCAASGLTFSNYWMNW | VH | huG14-117 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 143 | DVVMTQTPLTLSVTIGQPASISCKSSHSLLYSNGRTYLN | VL | huG14-118 |
| | WLLQRPGQSPKRLIYLVSKLDSGVPDRVTGSGSGTDFT | | |
| | LKISRVEAEDLGVYYCIQGTYFPYTFGGGTKLEIK | | |
| 144 | EVQLVESGGGLVKPGGSLRLSCAASGLTFSNYWMNW | VH | huG14-118 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 145 | DVVMTQTPLTLSVTIGQPASISCKSSHSLLYSNGETYLN | VL | huG14-119 |
| | WLLQRPGQSPKRLIYLVSKLDSGVPDRVTGSGSGTDFT | | |
| | LKISRVEAEDLGVYYCIQGTYFPYTFGGGTKLEIK | | |
| 146 | EVQLVESGGGLVKPGGSLRLSCAASGLTFSNYWGNW | VH | huG14-119 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 147 | DVVMTQTPLTLSVTIGQPASISCKSSHSLLYSNGKTYLN | VL | huG14-120 |
| | WLLQRPGQSPKRLIYLVSKLDSGVPDRVTGSGSGTDFT | | |
| | LKISRVEAEDLGVYYCVQGTYFPYTFGGGTKLEIK | | |
| 148 | EVQLVESGGGLVKPGGSLRLSCAASGLTFSNYWGNW | VH | huG14-120 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 149 | DVVMTQTPLTLSVTIGQPASISCKSSHSLLYSNGRTYLN | VL | huG14-121 |
| | WLLQRPGQSPKRLIYLVSKLDSGVPDRVTGSGSGTDFT | | |
| | LKISRVEAEDLGVYYCVQGTYFPYTFGGGTKLEIK | | |
| 150 | EVQLVESGGGLVKPGGSLRLSCAASGLTFSNYWGNW | VH | huG14-121 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 151 | DVVMTQTPLTLSVTIGQPASISCKSSQSLLYSNGKTYLN | VL | huG14-122 |
| | WLLQRPGQSPKRLIYLVSKLDSGVPDRVTGSGSGTDFT | | |
| | LKISRVEAEDLGVYYCIQGTYFPYTFGGGTKLEIK | | |
| 152 | EVQLVESGGGLVKPGGSLRLSCAASGLTFSNYWGNW | VH | huG14-122 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 153 | DVVMTQTPLTLSVTIGQPASISCKSSQSLLYSNGRTYLN | VL | huG14-123 |
| | WLLQRPGQSPKRLIYLVSKLDSGVPDRVTGSGSGTDFT | | |
| | LKISRVEAEDLGVYYCIQGTYFPYTFGGGTKLEIK | | |
| 154 | EVQLVESGGGLVKPGGSLRLSCAASGLTFSNYWGNW | VH | huG14-123 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 155 | DVVMTQTPLTLSVTIGQPASISCKSSHSLLYSNGKTYLN | VL | huG14-124 |
| | WLLQRPGQSPKRLIYLVSKLDSGVPDRVTGSGSGTDFT | | |
| | LKISRVEAEDLGVYYCIQGTYFPYTFGGGTKLEIK | | |
| 156 | EVQLVESGGGLVKPGGSLRLSCAASGLTFSNYWGNW | VH | huG14-124 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 157 | DVVMTQTPLTLSVTIGQPASISCKSSHSLLYSNGRTYLN | VL | huG14-125 |
| | WLLQRPGQSPKRLIYLVSKLDSGVPDRVTGSGSGTDFT | | |
| | LKISRVEAEDLGVYYCIQGTYFPYTFGGGTKLEIK | | |
| 158 | EVQLVESGGGLVKPGGSLRLSCAASGLTFSNYWGNW | VH | huG14-125 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 159 | DVVMTQTPLTLSVTIGQPASISCKSSHSLLYSNGETYLN | VL | huG14-126 |
| | WLLQRPGQSPKRLIYLVSKLDSGVPDRVTGSGSGTDFT | | |
| | LKISRVEAEDLGVYYCIQGTYFPYTFGGGTKLEIK | | |
| 160 | EVQLVESGGGLVKPGGSLRLSCAASGLTFSNYWMNW | VH | huG14-126 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 161 | DVVMTQTPLTLSVTIGQPASISCKSSHSLLYSNGKTYLN | VL | huG14-127 |
| | WLLQRPGQSPKRLIYLVSKLDSGVPDRVTGSGSGTDFT | | |
| | LKISRVEAEDLGVYYCVQGTYFPYTFGGGTKLEIK | | |
| 162 | EVQLVESGGGLVKPGGSLRLSCAASGLTFSNYWMNW | VH | huG14-127 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 163 | DVVMTQTPLTLSVTIGQPASISCKSSHSLLYSNGRTYLN | VL | huG14-128 |
| | WLLQRPGQSPKRLIYLVSKLDSGVPDRVTGSGSGTDFT | | |
| | LKISRVEAEDLGVYYCVQGTYFPYTFGGGTKLEIK | | |
| 164 | EVQLVESGGGLVKPGGSLRLSCAASGLTFSNYWMNW | VH | huG14-128 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 165 | DVVMTQTPLTLSVTIGQPASISCKSSQSLLYSNGKTYLN | VL | huG14-129 |
| | WLLQRPGQSPKRLIYLVSKLDSGVPDRVTGSGSGTDFT | | |
| | LKISRVEAEDLGVYYCIQGTYFPYTFGGGTKLEIK | | |
| 166 | EVQLVESGGGLVKPGGSLRLSCAASGLTFSNYWMNW | VH | huG14-129 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 167 | DVVMTQTPLTLSVTIGQPASISCKSSQSLLYSNGRTYLN | VL | huG14-130 |
| | WLLQRPGQSPKRLIYLVSKLDSGVPDRVTGSGSGTDFT | | |
| | LKISRVEAEDLGVYYCIQGTYFPYTFGGGTKLEIK | | |
| 168 | EVQLVESGGGLVKPGGSLRLSCAASGLTFSNYWMNW | VH | huG14-130 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 169 | DVVMTQTPLTLSVTIGQPASISCKSSHSLLYSNGKTYLN | VL | huG14-131 |
| | WLLQRPGQSPKRLIYLVSKLDSGVPDRVTGSGSGTDFT | | |
| | LKISRVEAEDLGVYYCIQGTYFPYTFGGGTKLEIK | | |
| 170 | EVQLVESGGGLVKPGGSLRLSCAASGLTFSNYWMNW | VH | huG14-131 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 171 | DVVMTQTPLTLSVTIGQPASISCKSSHSLLYSNGRTYLN | VL | huG14-132 |
| | WLLQRPGQSPKRLIYLVSKLDSGVPDRVTGSGSGTDFT | | |
| | LKISRVEAEDLGVYYCIQGTYFPYTFGGGTKLEIK | | |
| 172 | EVQLVESGGGLVKPGGSLRLSCAASGLTFSNYWMNW | VH | huG14-132 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 173 | DVVMTQTPLTLSVTIGQPASISCKSSHSLLYSNGETYLN | VL | huG14-133 |
| | WLLQRPGQSPKRLIYLVSKLDSGVPDRVTGSGSGTDFT | | |
| | LKISRVEAEDLGVYYCIQGTYFPYTFGGGTKLEIK | | |
| 174 | EVQLVESGGGLVKPGGSLRLSCAASGLTFSNYWGNW | VH | huG14-133 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 175 | DVVMTQTPLTLSVTIGQPASISCKSSHSLLYSNGKTYLN | VL | huG14-134 |
| | WLLQRPGQSPKRLIYLVSKLDSGVPDRVTGSGSGTDFT | | |
| | LKISRVEAEDLGVYYCVQGTYFPYTFGGGTKLEIK | | |
| 176 | EVQLVESGGGLVKPGGSLRLSCAASGLTFSNYWGNW | VH | huG14-134 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 177 | DVVMTQTPLTLSVTIGQPASISCKSSHSLLYSNGRTYLN | VL | huG14-135 |
| | WLLQRPGQSPKRLIYLVSKLDSGVPDRVTGSGSGTDFT | | |
| | LKISRVEAEDLGVYYCVQGTYFPYTFGGGTKLEIK | | |
| 178 | EVQLVESGGGLVKPGGSLRLSCAASGLTFSNYWGNW | VH | huG14-135 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 179 | DVVMTQTPLTLSVTIGQPASISCKSSQSLLYSNGKTYLN | VL | huG14-136 |
| | WLLQRPGQSPKRLIYLVSKLDSGVPDRVTGSGSGTDFT | | |
| | LKISRVEAEDLGVYYCIQGTYFPYTFGGGTKLEIK | | |
| 180 | EVQLVESGGGLVKPGGSLRLSCAASGLTFSNYWGNW | VH | huG14-136 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 181 | DVVMTQTPLTLSVTIGQPASISCKSSQSLLYSNGRTYLN | VL | huG14-137 |
| | WLLQRPGQSPKRLIYLVSKLDSGVPDRVTGSGSGTDFT | | |
| | LKISRVEAEDLGVYYCIQGTYFPYTFGGGTKLEIK | | |
| 182 | EVQLVESGGGLVKPGGSLRLSCAASGLTFSNYWGNW | VH | huG14-137 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 183 | DVVMTQTPLTLSVTIGQPASISCKSSHSLLYSNGKTYLN | VL | huG14-138 |
| | WLLQRPGQSPKRLIYLVSKLDSGVPDRVTGSGSGTDFT | | |
| | LKISRVEAEDLGVYYCIQGTYFPYTFGGGTKLEIK | | |
| 184 | EVQLVESGGGLVKPGGSLRLSCAASGLTFSNYWGNW | VH | huG14-138 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 185 | DVVMTQTPLTLSVTIGQPASISCKSSHSLLYSNGRTYLN | VL | huG14-139 |
| | WLLQRPGQSPKRLIYLVSKLDSGVPDRVTGSGSGTDFT | | |
| | LKISRVEAEDLGVYYCIQGTYFPYTFGGGTKLEIK | | |
| 186 | EVQLVESGGGLVKPGGSLRLSCAASGLTFSNYWGNW | VH | huG14-139 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 187 | DVVMTQTPLTLSVTIGQPASISCKSSHSLLYSNHKTYLN | VL | huG14-124- |
| | WLLQRPGQSPKRLIYLVSKLDSGVPDRVTGSGSGTDFT | | 1 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 1 |
| | DDSKSTVYLQMNSLKTEDTAVYYCTTGTDYWGQGTT | | |
| | VTVSS | | |
| 189 | DVVMTQTPLTLSVTIGQPASISCKSSHSLLYSNKKTYLN | VI | huG14-124- |
| | WLLQRPGQSPKRLIYLVSKLDSGVPDRVTGSGSGTDFT | | 2 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 2 |
| | DDSKSTVYLQMNSLKTEDTAVYYCTTGTDYWGQGTT | | |
| | VTVSS | | |
| 190 | DVVMTQTPLTLSVTIGQPASISCKSSHSLLYSNRKTYLN | VL | huG14-124- |
| | WLLQRPGQSPKRLIYLVSKLDSGVPDRVTGSGSGTDFT | | 3 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 3 |
| | DDSKSTVYLQMNSLKTEDTAVYYCTTGTDYWGQGTT | | |
| | VTVSS | | |
| 191 | DVVMTQTPLTLSVTIGQPASISCKSSHSLLYSNWKTYL | VL | huG14-124- |
| | NWLLQRPGQSPKRLIYLVSKLDSGVPDRVTGSGSGTDF | | 4 |
| | TLKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 4 |
| | DDSKSTVYLQMNSLKTEDTAVYYCTTGTDYWGQGTT | | |
| | VTVSS | | |
| 192 | DVVMTQTPLTLSVTIGQPASISCKSSHSLLYSNYKTYLN | VL | huG14-124- |
| | WLLQRPGQSPKRLIYLVSKLDSGVPDRVTGSGSGTDFT | | 5 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 5 |
| | DDSKSTVYLQMNSLKTEDTAVYYCTTGTDYWGQGTT | | |
| | VTVSS | | |
| 193 | DVVMTQTPLTLSVTIGQPASISCKSSHSLLYSNAKTYLN | VL | huG14-124- |
| | WLLQRPGQSPKRLIYLVSKLDSGVPDRVTGSGSGTDFT | | 6 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 6 |
| | DDSKSTVYLQMNSLKTEDTAVYYCTTGTDYWGQGTT | | |
| | VTVSS | | |
| 194 | DVVMTQTPLTLSVTIGQPASISCKSSHSLLYSYGKTYLN | VI | huG14-124- |
| | WLLQRPGQSPKRLIYLVSKLDSGVPDRVTGSGSGTDFT | | 7 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 7 |
| | DDSKSTVYLQMNSLKTEDTAVYYCTTGTDYWGQGTT | | |
| | VTVSS | | |
| 195 | DVVMTQTPLTLSVTIGQPASISCKSSHSLLYSQGKTYLN | VL | huG14-124- |
| | WLLQRPGQSPKRLIYLVSKLDSGVPDRVTGSGSGTDFT | | 8 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 8 |
| | DDSKSTVYLQMNSLKTEDTAVYYCTTGTDYWGQGTT | | |
| | VTVSS | | |
| 196 | DVVMTQTPLTLSVTIGQPASISCKSSHSLLYSSGKTYLN | VL | huG14-124- |
| | WLLQRPGQSPKRLIYLVSKLDSGVPDRVTGSGSGTDFT | | 9 |
| | LKISRVEAEDLGVYYCIQGTYFPYTFGGGTKLEIK | | |
| 188 | EVQLVESGGGLVKPGGSLRLSCAASGLTFSNYWGNW | VH | huG14-124- |
| | VRQAPGKGLEWVALIRLYSDNYAIHYAESVKGRFTISR | | 9 |
| | DDSKSTVYLQMNSLKTEDTAVYYCTTGTDYWGQGTT | | |
| | VTVSS | | |
| 201 | DVVMTQSPLSLPVTPGEPASISCKSSQSLLYSNGETYLN | VL | huG14-2 |
| | WLLQKPGQSPQRLIYLVSKLDSGVPDRFSGSGSGTDFT | | |
| | LKISRVEAEDLGVYYCVQGTYFPYTFGQGTKLEIK | | |
| 202 | EVQLVESGGGLVKPGGSLRLSCAASGLTFSNYWMNW | VH | huG14-2 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 203 | KSSQSLLYSNGETYLN | LCDR1 | huG14-2 |
| 204 | LVSKLDS | LCDR2 | huG14-2 |
| 205 | VQGTYFPYT | LCDR3 | huG14-2 |
| 206 | NYWMN | HCDR1 | huG14-2 |
| 207 | LIRLYSDNYAIHYAESVKG | HCDR2 | huG14-2 |
| 208 | GTDY | HCDR3 | huG14-2 |
| 209 | DVVMTQSPLSLPVTPGRPASISCKSSQSLLYSNGETYLN | VL | huG14-201 |
| | WLLQKPGQSPQRLIYLVSKLDSGVPDRFSGSGSGTDFT | | |
| | LKISRVEAEDLGVYYCVQGTYFPYTFGQGTKLEIK | | |
| 210 | EVQLVESGGGLVKPGGSLRLSCAASGLTFSNYWMNW | VH | huG14-201 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 211 | DVVMTQSPLSLYVTPGEPASISCKSSQSLLYSNGETYLN | VL | huG14-202 |
| | WLLQKPGQSPQRLIYLVSKLDSGVPDRFSGSGSGTDFT | | |
| | LKISRVEAEDLGVYYCVQGTYFPYTFGQGTKLEIK | | |
| 212 | EVQLVESGGGLVKPGGSLRLSCAASGLTFSNYWMNW | VH | huG14-202 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 213 | DVVMTQSPLSLPVTPGEPASISCKSSQSLLYSNGETYLN | VL | huG14-203 |
| | WLLQKPGQSPQRLIYLVSKLDSGVPDRFSGSGSGTDFT | | |
| | LKISRVEAEDLGVYYCVQGTYFPYTFGIGTKLEIK | | |
| 214 | EVQLVESGGGLVKPGGSLRLSCAASGLTFSNYWMNW | VH | huG14-203 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 215 | DVVMTQSPLSLPVTPGRPASISCKSSHSLLYSNGETYLN | VL | huG14-204 |
| | WLLQKPGQSPQRLIYLVSKLDSGVPDRFSGSGSGTDFT | | |
| | LKISRVEAEDLGVYYCIQGTYFPYTFGQGTKLEIK | | |
| 216 | EVQLVESGGGLVKPGGSLRLSCAASGLTFSNYWMNW | VH | huG14-204 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 217 | DVVMTQSPLSLPVTPGRPASISCKSSHSLLYSNGKTYLN | VL | huG14-205 |
| | WLLQKPGQSPQRLIYLVSKLDSGVPDRFSGSGSGTDFT | | |
| | LKISRVEAEDLGVYYCVQGTYFPYTFGQGTKLEIK | | |
| 218 | EVQLVESGGGLVKPGGSLRLSCAASGLTFSNYWMNW | VH | huG14-205 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 219 | DVVMTQSPLSLPVTPGRPASISCKSSHSLLYSNGRTYLN | VL | huG14-206 |
| | WLLQKPGQSPQRLIYLVSKLDSGVPDRFSGSGSGTDFT | | |
| | LKISRVEAEDLGVYYCVQGTYFPYTFGQGTKLEIK | | |
| 220 | EVQLVESGGGLVKPGGSLRLSCAASGLTFSNYWMNW | VH | huG14-206 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 221 | DVVMTQSPLSLPVTPGRPASISCKSSQSLLYSNGKTYLN | VL | huG14-207 |
| | WLLQKPGQSPQRLIYLVSKLDSGVPDRFSGSGSGTDFT | | |
| | LKISRVEAEDLGVYYCIQGTYFPYTFGQGTKLEIK | | |
| 222 | EVQLVESGGGLVKPGGSLRLSCAASGLTFSNYWMNW | VH | huG14-207 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 223 | DVVMTQSPLSLPVTPGRPASISCKSSQSLLYSNGRTYLN | VL | huG14-208 |
| | WLLQKPGQSPQRLIYLVSKLDSGVPDRFSGSGSGTDFT | | |
| | LKISRVEAEDLGVYYCIQGTYFPYTFGQGTKLEIK | | |
| 224 | EVQLVESGGGLVKPGGSLRLSCAASGLTFSNYWMNW | VH | huG14-208 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 225 | DVVMTQSPLSLPVTPGRPASISCKSSHSLLYSNGKTYLN | VL | huG14-209 |
| | WLLQKPGQSPQRLIYLVSKLDSGVPDRFSGSGSGTDFT | | |
| | LKISRVEAEDLGVYYCIQGTYFPYTFGQGTKLEIK | | |
| 226 | EVQLVESGGGLVKPGGSLRLSCAASGLTFSNYWMNW | VH | huG14-209 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 227 | DVVMTQSPLSLPVTPGRPASISCKSSHSLLYSNGRTYLN | VL | huG14-210 |
| | WLLQKPGQSPQRLIYLVSKLDSGVPDRFSGSGSGTDFT | | |
| | LKISRVEAEDLGVYYCIQGTYFPYTFGQGTKLEIK | | |
| 228 | EVQLVESGGGLVKPGGSLRLSCAASGLTFSNYWMNW | VH | huG14-210 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 229 | DVVMTQSPLSLPVTPGRPASISCKSSHSLLYSNGETYLN | VL | huG14-211 |
| | WLLQKPGQSPQRLIYLVSKLDSGVPDRFSGSGSGTDFT | | |
| | LKISRVEAEDLGVYYCIQGTYFPYTFGQGTKLEIK | | |
| 230 | EVQLVESGGGLVKPGGSLRLSCAASGLTFSNYWGNW | VH | huG14-211 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 231 | DVVMTQSPLSLPVTPGRPASISCKSSHSLLYSNGKTYLN | VL | huG14-212 |
| | WLLQKPGQSPQRLIYLVSKLDSGVPDRFSGSGSGTDFT | | |
| | LKISRVEAEDLGVYYCVQGTYFPYTFGQGTKLEIK | | |
| 232 | EVQLVESGGGLVKPGGSLRLSCAASGLTFSNYWGNW | VH | huG14-212 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 233 | DVVMTQSPLSLPVTPGRPASISCKSSHSLLYSNGRTYLN | VL | huG14-213 |
| | WLLQKPGQSPQRLIYLVSKLDSGVPDRFSGSGSGTDFT | | |
| | LKISRVEAEDLGVYYCVQGTYFPYTFGQGTKLEIK | | |
| 234 | EVQLVESGGGLVKPGGSLRLSCAASGLTFSNYWGNW | VH | huG14-213 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 235 | DVVMTQSPLSLPVTPGRPASISCKSSQSLLYSNGKTYLN | VL | huG14-214 |
| | WLLQKPGQSPQRLIYLVSKLDSGVPDRFSGSGSGTDFT | | |
| | LKISRVEAEDLGVYYCIQGTYFPYTFGQGTKLEIK | | |
| 236 | EVQLVESGGGLVKPGGSLRLSCAASGLTFSNYWGNW | VH | huG14-214 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 237 | DVVMTQSPLSLPVTPGRPASISCKSSQSLLYSNGRTYLN | VL | huG14-215 |
| | WLLQKPGQSPQRLIYLVSKLDSGVPDRFSGSGSGTDFT | | |
| | LKISRVEAEDLGVYYCIQGTYFPYTFGQGTKLEIK | | |
| 238 | EVQLVESGGGLVKPGGSLRLSCAASGLTFSNYWGNW | VH | huG14-215 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 239 | DVVMTQSPLSLPVTPGRPASISCKSSHSLLYSNGKTYLN | VL | huG14-216 |
| | WLLQKPGQSPQRLIYLVSKLDSGVPDRFSGSGSGTDFT | | |
| | LKISRVEAEDLGVYYCIQGTYFPYTFGQGTKLEIK | | |
| 240 | EVQLVESGGGLVKPGGSLRLSCAASGLTFSNYWGNW | VH | huG14-216 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 241 | DVVMTQSPLSLPVTPGRPASISCKSSHSLLYSNGRTYLN | VL | huG14-217 |
| | WLLQKPGQSPQRLIYLVSKLDSGVPDRFSGSGSGTDFT | | |
| | LKISRVEAEDLGVYYCIQGTYFPYTFGQGTKLEIK | | |
| 242 | EVQLVESGGGLVKPGGSLRLSCAASGLTFSNYWGNW | VH | huG14-217 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 243 | DVVMTQSPLSLPVTPGRPASISCKSSHSLLYSNGETYLN | VL | huG14-218 |
| | WLLQKPGQSPQRLIYLVSKLDSGVPDRFSGSGSGTDFT | | |
| | LKISRVEAEDLGVYYCIQGTYFPYTFGQGTKLEIK | | |
| 244 | EVQLVESGGGLVKPGGSLRLSCAASGLTFSNYWMNW | VH | huG14-218 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 245 | DVVMTQSPLSLPVTPGRPASISCKSSHSLLYSNGKTYLN | VL | huG14-219 |
| | WLLQKPGQSPQRLIYLVSKLDSGVPDRFSGSGSGTDFT | | |
| | LKISRVEAEDLGVYYCVQGTYFPYTFGQGTKLEIK | | |
| 246 | EVQLVESGGGLVKPGGSLRLSCAASGLTFSNYWMNW | VH | huG14-219 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 247 | DVVMTQSPLSLPVTPGRPASISCKSSHSLLYSNGRTYLN | VL | huG14-220 |
| | WLLQKPGQSPQRLIYLVSKLDSGVPDRFSGSGSGTDFT | | |
| | LKISRVEAEDLGVYYCVQGTYFPYTFGQGTKLEIK | | |
| 248 | EVQLVESGGGLVKPGGSLRLSCAASGLTFSNYWMNW | VH | huG14-220 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 249 | DVVMTQSPLSLPVTPGRPASISCKSSQSLLYSNGKTYLN | VL | huG14-221 |
| | WLLQKPGQSPQRLIYLVSKLDSGVPDRFSGSGSGTDFT | | |
| | LKISRVEAEDLGVYYCIQGTYFPYTFGQGTKLEIK | | |
| 250 | EVQLVESGGGLVKPGGSLRLSCAASGLTFSNYWMNW | VH | huG14-221 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 251 | DVVMTQSPLSLPVTPGRPASISCKSSQSLLYSNGRTYLN | VL | huG14-222 |
| | WLLQKPGQSPQRLIYLVSKLDSGVPDRFSGSGSGTDFT | | |
| | LKISRVEAEDLGVYYCIQGTYFPYTFGQGTKLEIK | | |
| 252 | EVQLVESGGGLVKPGGSLRLSCAASGLTFSNYWMNW | VH | huG14-222 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 253 | DVVMTQSPLSLPVTPGRPASISCKSSHSLLYSNGKTYLN | VL | huG14-223 |
| | WLLQKPGQSPQRLIYLVSKLDSGVPDRFSGSGSGTDFT | | |
| | LKISRVEAEDLGVYYCIQGTYFPYTFGQGTKLEIK | | |
| 254 | EVQLVESGGGLVKPGGSLRLSCAASGLTFSNYWMNW | VH | huG14-223 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 255 | DVVMTQSPLSLPVTPGRPASISCKSSHSLLYSNGRTYLN | VL | huG14-224 |
| | WLLQKPGQSPQRLIYLVSKLDSGVPDRFSGSGSGTDFT | | |
| | LKISRVEAEDLGVYYCIQGTYFPYTFGQGTKLEIK | | |
| 256 | EVQLVESGGGLVKPGGSLRLSCAASGLTFSNYWMNW | VH | huG14-224 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 257 | DVVMTQSPLSLPVTPGRPASISCKSSHSLLYSNGETYLN | VL | huG14-225 |
| | WLLQKPGQSPQRLIYLVSKLDSGVPDRFSGSGSGTDFT | | |
| | LKISRVEAEDLGVYYCIQGTYFPYTFGQGTKLEIK | | |
| 258 | EVQLVESGGGLVKPGGSLRLSCAASGLTFSNYWGNW | VH | huG14-225 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 259 | DVVMTQSPLSLPVTPGRPASISCKSSHSLLYSNGKTYLN | VL | huG14-226 |
| | WLLQKPGQSPQRLIYLVSKLDSGVPDRFSGSGSGTDFT | | |
| | LKISRVEAEDLGVYYCVQGTYFPYTFGQGTKLEIK | | |
| 260 | EVQLVESGGGLVKPGGSLRLSCAASGLTFSNYWGNW | VH | huG14-226 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 261 | DVVMTQSPLSLPVTPGRPASISCKSSHSLLYSNGRTYLN | VL | huG14-227 |
| | WLLQKPGQSPQRLIYLVSKLDSGVPDRFSGSGSGTDFT | | |
| | LKISRVEAEDLGVYYCVQGTYFPYTFGQGTKLEIK | | |
| 262 | EVQLVESGGGLVKPGGSLRLSCAASGLTFSNYWGNW | VH | huG14-227 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 263 | DVVMTQSPLSLPVTPGRPASISCKSSQSLLYSNGKTYLN | VL | huG14-228 |
| | WLLQKPGQSPQRLIYLVSKLDSGVPDRFSGSGSGTDFT | | |
| | LKISRVEAEDLGVYYCIQGTYFPYTFGQGTKLEIK | | |
| 264 | EVQLVESGGGLVKPGGSLRLSCAASGLTFSNYWGNW | VH | huG14-228 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 265 | DVVMTQSPLSLPVTPGRPASISCKSSQSLLYSNGRTYLN | VL | huG14-229 |
| | WLLQKPGQSPQRLIYLVSKLDSGVPDRFSGSGSGTDFT | | |
| | LKISRVEAEDLGVYYCIQGTYFPYTFGQGTKLEIK | | |
| 266 | EVQLVESGGGLVKPGGSLRLSCAASGLTFSNYWGNW | VH | huG14-229 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 267 | DVVMTQSPLSLPVTPGRPASISCKSSHSLLYSNGKTYLN | VL | huG14-230 |
| | WLLQKPGQSPQRLIYLVSKLDSGVPDRFSGSGSGTDFT | | |
| | LKISRVEAEDLGVYYCIQGTYFPYTFGQGTKLEIK | | |
| 268 | EVQLVESGGGLVKPGGSLRLSCAASGLTFSNYWGNW | VH | huG14-230 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 269 | DVVMTQSPLSLPVTPGRPASISCKSSHSLLYSNGRTYLN | VL | huG14-231 |
| | WLLQKPGQSPQRLIYLVSKLDSGVPDRFSGSGSGTDFT | | |
| | LKISRVEAEDLGVYYCIQGTYFPYTFGQGTKLEIK | | |
| 270 | EVQLVESGGGLVKPGGSLRLSCAASGLTFSNYWGNW | VH | huG14-231 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 271 | DVVMTQSPLSLYVTPGRPASISCKSSHSLLYSNGRTYL | VL | huG14-232 |
| | NWLLQKPGQSPQRLIYLVSKLDSGVPDRFSGSGSGTDF | | |
| | TLKISRVEAEDLGVYYCIQGTYFPYTFGQGTKLEIK | | |
| 272 | EVQLVESGGGLVKPGGSLRLSCAASGLTFSNYWGNW | VH | huG14-232 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 273 | DVVMTQSPLRLPVTPGRPASISCKSSHSLLYSNGRTYL | VL | huG14-233 |
| | NWLLQKPGQSPQRLIYLVSKLDSGVPDRFSGSGSGTDF | | |
| | TLKISRVEAEDLGVYYCIQGTYFPYTFGIGTKLEIK | | |
| 274 | EVQLVESGGGLVKPGGSLRLSCAASGLTFSNYWGNW | VH | huG14-233 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 275 | DVVMTQSPLSLPVTPGRPASISCKSSHSLLYSNKKTYLN | VL | huG14-234 |
| | WLLQKPGQSPQRLIYLVSKLDSGVPDRFSGSGSGTDFT | | |
| | LKISRVEAEDLGVYYCIQGTYFPYTFGQGTKLEIK | | |
| 276 | EVQLVESGGGLVKPGGSLRLSCAASGLTFSNYWGNW | VH | huG14-234 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 277 | DVVMTQSPLSLYVTPGRPASISCKSSHSLLYSNKRTYL | VL | huG14-235 |
| | NWLLQKPGQSPQRLIYLVSKLDSGVPDRFSGSGSGTDF | | |
| | TLKISRVEAEDLGVYYCIQGTYFPYTFGQGTKLEIK | | |
| 278 | EVQLVESGGGLVKPGGSLRLSCAASGLTFSNYWGNW | VH | huG14-235 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 279 | DVVMTQSPLSLYVTPGRPASISCKSSQSLLYSNGETYL | VL | huG14-2011 |
| | NWLLQKPGQSPQRLIYLVSKLDSGVPDRFSGSGSGTDF | | |
| | TLKISRVEAEDLGVYYCVQGTYFPYTFGQGTKLEIK | | |
| 280 | EVQLVESGGGLVKPGGSLRLSCAASGLTFSNYWMNW | VH | huG14-2011 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 281 | DVVMTQSPLRLYVTPGRPASISCKSSQSLLYSNGETYL | VL | huG14-2012 |
| | NWLLQKPGQSPQRLIYLVSKLDSGVPDRFSGSGSGTDF | | |
| | TLKISRVEAEDLGVYYCVQGTYFPYTFGIGTKLEIK | | |
| 282 | EVQLVESGGGLVKPGGSLRLSCAASGLTFSNYWMNW | VH | huG14-2012 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 301 | DVVMTQSPLSLPVTLGQPASISCKSSQSLLYSNGETYLN | VL | huG14-3 |
| | WLQQRPGQSPRRLIYLVSKLDSGVPDRFSGSGSGTDFT | | |
| | LKISRVEAEDLGVYYCVQGTYFPYTFGQGTKLEIK | | |
| 302 | EVQLVESGGGLVKPGGSLRLSCAASGLTFSNYWMNW | VH | huG14-3 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 303 | KSSQSLLYSNGETYLN | LCDR1 | huG14-3 |
| 304 | LVSKLDS | LCDR2 | huG14-3 |
| 305 | VQGTYFPYT | LCDR3 | huG14-3 |
| 306 | NYWMN | HCDR1 | huG14-3 |
| 307 | LIRLYSDNYAIHYAESVKG | HCDR2 | huG14-3 |
| 308 | GTDY | HCDR3 | huG14-3 |
| 309 | DVVMTQSPLSLPVTLGQPASISCKSSHSLLYSNKRTYL | VL | huG14-301 |
| | NWLQQRPGQSPRRLIYLVSKLDSGVPDRFSGSGSGTDF | | |
| | TLKISRVEAEDLGVYYCIQGTYFPYTFGQGTKLEIK | | |
| 310 | EVQLVESGGGLVKPGGSLRLSCAASGLTFSNYWGNW | VH | huG14-301 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 311 | DVVMTQSPLSLPVTLGQPASISCKSSHSLLYSNKRTYL | VL | huG14-302 |
| | NWLQQRPGQSPRRLIYLVSKLDSGVPDRFSGSGSGTDF | | |
| | TLKISRVEAEDLGVYYCIQGTYFPYTFGQGTKLEIK | | |
| 312 | EVQLVESGGGLVKPGGSLRLSCAASGLTFSNYWGNW | VH | huG14-302 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 313 | DVVMTQSPLSLPVTLGQPASISCKSSHSLLYSNKKTYL | VL | huG14-303 |
| | NWLQQRPGQSPRRLIYLVSKLDSGVPDRFSGSGSGTDF | | |
| | TLKISRVEAEDLGVYYCIQGTYFPYTFGQGTKLEIK | | |
| 314 | EVQLVESGGGLVKPGGSLRLSCAASGLTFSNYWGNW | VH | huG14-303 |
| | VRQAPGKGLEWVALIRLYSDNYAIHYAESVKGRFTISR | | |
| | DDSKSTVYLQMNSLKTEDTAVYYCTTGTDYWGQGTT | | |
| | VTVSS | | |
| 315 | DVVMTQSPLSLPVTLGQPASISCKSSHSLLYSNKRTYL | VL | huG14-304 |
| | NWLQQRPGQSPRRLIYLVSKLDSGVPDRFSGSGSGTDF | | |
| | TLKISRVEAEDLGVYYCIQGTYFPYTFGQGTKLEIK | | |
| 316 | EVQLVESGGGLVKPGGSLRLSCAASGLTFSNYWMNW | VH | huG14-304 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 317 | DVVMTQSPLSLPVTLGQPASISCKSSHSLLYSNKKTYL | VL | huG14-305 |
| | NWLQQRPGQSPRRLIYLVSKLDSGVPDRFSGSGSGTDF | | |
| | TLKISRVEAEDLGVYYCIQGTYFPYTFGQGTKLEIK | | |
| 318 | EVQLVESGGGLVKPGGSLRLSCAASGLTFSNYWMNW | VH | huG14-305 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 319 | DVVMTQSPLSLPVTLGQPASISCKSSHSLLYSNKKTYL | VL | huG14-306 |
| | NWLQQRPGQSPRRLIYLVSKLDSGVPDRFSGSGSGTDF | | |
| | TLKISRVEAEDLGVYYCIQGTYFPYTFGQGTKLEIK | | |
| 320 | EVQLVESGGGLVKPGGSLRLSCAASGLTFSNYWGNW | VH | huG14-306 |
| | VRQAPGKGLEWVALIRLYSDNYAIHYAESVKGRFTISR | | |
| | DDSKSTVYLQMNSLKTEDTAVYYCTTGTDNWGQGTT | | |
| | VTVSS | | |
| 401 | KSSX1SLLYSX2X3X4TYLN | LCDR1 | |
| 402 | KSSX1SLLYSX2X3X4TYLN; wherein X1 is Q, | LCDR1 | |
| | F, or H; X2 is N, Y, Q or S; X3 is G, W, | | |
| | H, K, R, A or Y; X4 is E, I, K, R, | | |
| | or V | | |
| 403 | LVSKLDS | LCDR2 | |
| 404 | Blank | | |
| 405 | XQGTYFPYT | LCDR3 | |
| 406 | XQGTYFPYT; wherein X is V or I | LCDR3 | |
| 407 | NYWXN | HCDR1 | |
| 408 | NYWXN; wherein X is M or G | HCDR1 | |
| 409 | LIRLYSDNYAIHYAESVKG | HCDR2 | |
| 410 | Blank | | |
| 411 | GTDX | HCDR3 | |
| 412 | GTDX; wherein X is Y, N, or F | HCDR3 | |
| 500 | SETTTGTSSNSSQSTSNSGLAPNPTNATTKAAG | mature | |
| | | human | |
| | | CD24 | |
| | | (27-59aa) | |
| 501 | SGLAP | epitope | |
| 502 | MGRAMVARLGLGLLLLALLLPTQIYSSETTTGTSSNSS | human | |
| | QSTSNSGLAPNPTNATTKAAGGALQSTASLFVVSLSLL | CD24 | |
| | HLYS | (with | |
| | | signal | |
| | | peptide) | |
| 503 | MSETTTGTSSNSSQSTSNSGLAPNPTNATTKSPILGYWK | CD24 (27- | |
| | IKGLVQPTRLLLEYLEEKYEEHLYERDEGDKWRNKKF | 56aa)- | |
| | ELGLEFPNLPYYIDGDVKLTQSMAIIRYIADKHNMLGG | GST, | |
| | CPKERAEISMLEGAVLDIRYGVSRIAYSKDFETLKVDFL | NEOX-2 | |
| | SKLPEMLKMFEDRLCHKTYLNGDHVTHPDFMLYDAL | | |
| | DVVLYMDPMCLDAFPKLVCFKKRIEAIPQIDKYLKSSK | | |
| | YIAWPLQGWQATFGGGDHPPKSD | | |
| 504 | MSETTTGTSSNSSQSTSNSGLAPNPTNATTKAAMSDSE | CD24(27- | |
| | VNQEAKPEVKPEVKPETHINLKVSDGSSEIFFKIKKTTP | 56aa)- | |
| | LRRLMEAFAKRQGKEMDSLRFLYDGIRIQADQTPEDL | SUMO, | |
| | DMEDNDIIEAHREQIGGHHHHHH | NEOX-27 | |
| 505 | MGSTAILGLLLAVLQGGRASETTTGTSSNSSQSTSNSGL | CD24(27- | |
| | APNPTNATTKSPILGYWKIKGLVQPTRLLLEYLEEKYE | 56aa)- | |
| | EHLYERDEGDKWRNKKFELGLEFPNLPYYIDGDVKLT | GST, | |
| | QSMAIIRYIADKHNMLGGCPKERAEISMLEGAVLDIRY | NEOX-4 | |
| | GVSRIAYSKDFETLKVDFLSKLPEMLKMFEDRLCHKTY | | |
| | LNGDHVTHPDFMLYDALDVVLYMDPMCLDAFPKLVC | | |
| | FKKRIEAIPQIDKYLKSSKYIAWPLQGWQATFGGGDHP | | |
| | PKSD | | |
| 601 | DIVMTQSPDSLNVSLGERATIRCRSSQSLLYSSDQKNYL | Reference | |
| | TWYQQKPGQPPKLLISWASPRASGVPDRFSGSGSGTDF | 1-SWA11- | |
| | TLTISSLQAEDVGVYYCQQYFIYPLTFGVGTKLELKRT | Light | |
| | VAAPSVFIFPPSDEQLKSGTASVVCLLNNFYPREAKVQ | chain | |
| | WKVDNALQSGNSQESVTEQDSKDSTYSLSSTLTLSKA | | |
| | DYEKHKVYACEVTHQGLSSPVTKSFNRGEC | | |
| 602 | QVHLQESGPGLVKPSQTLSLTCAVSGYSISSGYTWHWI | Reference | |
| | RQPPGKTVEWIGYIQYTGSTRYNPALRGRLTMSVDTSK | 1-SWA11- | |
| | NQFSLKLISVTAADTGVYFCARGTTASFDYWGQGTTV | Heavy | |
| | TVSSASTKGPSVFPLAPSSKSTSGGTAALGCLVKDYFPE | chain | |
| | PVTVSWNSGALTSGVHTFPAVLQSSGLYSLSSVVTVPS | | |
| | SSLGTQTYICNVNHKPSNTKVDKKVEPKSCDKTHTCPP | | |
| | CPAPELLGGPSVFLFPPKPKDTLMISRTPEVTCVVVDVS | | |
| | HEDPEVKFNWYVDGVEVHNAKTKPREEQYNSTYRVV | | |
| | SVLTVLHQDWLNGKEYKCKVSNKALPAPIEKTISKAK | | |
| | GQPREPQVYTLPPSREEMTKNQVSLTCLVKGFYPSDIA | | |
| | VEWESNGQPENNYKTTPPVLDSDGSFFLYSKLTVDKSR | | |
| | WQQGNVFSCSVMHEALHNHYTQKSLSLSPGK | | |
| 603 | DIVMTQTPLSLSVTIGQPASISCKSSQSLLYSNGKTYLN | Reference | |
| | WLQQRPGQSPKRLIYQVSKLDPGIPDRFSGSGSETDFTL | 2-P6373- | |
| | KISRVEAEDLGIYYCMQGSSLPWTFGGGTKLEIKRTVA | Light | |
| | APSVFIFPPSDEQLKSGTASVVCLLNNFYPREAKVQWK | chain | |
| | VDNALQSGNSQESVTEQDSKDSTYSLSSTLTLSKADYE | | |
| | KHKVYACEVTHQGLSSPVTKSFNRGEC | | |
| 604 | EVKFEESGGGLVQPGGSIKLSCAASGVTFSEAWMDWV | Reference | |
| | RQSPEKGLEWVAEIRDKPNSYVTYYAESVKGRFTISRD | 2-P6373- | |
| | DSKSRVYLQMNNLRTEDTGIYYCTGAMDYWGQGTSV | Heavy | |
| | TVSSASTKGPSVFPLAPSSKSTSGGTAALGCLVKDYFPE | chain | |
| | PVTVSWNSGALTSGVHTFPAVLQSSGLYSLSSVVTVPS | | |
| | SSLGTQTYICNVNHKPSNTKVDKKVEPKSCDKTHTCPP | | |
| | CPAPELLGGPSVFLFPPKPKDTLMISRTPEVTCVVVDVS | | |
| | HEDPEVKFNWYVDGVEVHNAKTKPREEQYNSTYRVV | | |
| | SVLTVLHQDWLNGKEYKCKVSNKALPAPIEKTISKAK | | |
| | GQPREPQVYTLPPSREEMTKNQVSLTCLVKGFYPSDIA | | |
| | VEWESNGQPENNYKTTPPVLDSDGSFFLYSKLTVDKSR | | |
| | WQQGNVFSCSVMHEALHNHYTQKSLSLSPGK | | |
| 605 | DIVMTQSPDSLAVSLGERATINCKSSQSLLYSSNQKNY | Reference | |
| | LAWYQQKPGQPPKLLIYWASTRESGVPDRFSGSGSGT | 3- | |
| | DFTLTISSLQAEDVAVYYCQQNFIYPLTFGGGTKVELK | IMM47H- | |
| | RTVAAPSVFIFPPSDEQLKSGTASVVCLLNNFYPREAK | Light | |
| | VQWKVDNALQSGNSQESVTEQDSKDSTYSLSSTLTLS | chain | |
| | KADYEKHKVYACEVTHQGLSSPVTKSFNRGEC | | |
| 606 | DVQLQESGPGLVKPSETLSLTCTVSGYSITSGYSWHWI | Reference | |
| | RQPPGKGLEWIGYIHYSGSTKYNPSLKSRVTISVDTSKN | 3- | |
| | QFSLKLSSVTAADTAVYYCARGADYALDYWGQRTSV | IMM47H- | |
| | TVSSASTKGPSVFPLAPSSKSTSGGTAALGCLVKDYFPE | Heavy | |
| | PVTVSWNSGALTSGVHTFPAVLQSSGLYSLSSVVTVPS | chain | |
| | SSLGTQTYICNVNHKPSNTKVDKRVEPKSCDKTHTCPP | | |
| | CPAPELLGGPSVFLFPPKPKDTLMISRTPEVTCVVVDVS | | |
| | HEDPEVKFNWYVDGVEVHNAKTKPREEQYNATYRVV | | |
| | SVLTVLHQDWLNGKEYKCKVSNKALPAPIAATISKAK | | |
| | GQPREPQVYTLPPSREEMTKNQVSLTCLVKGFYPSDIA | | |
| | VEWESNGQPENNYKTTPPVLDSDGSFFLYSKLTVDKSR | | |
| | WQQGNVFSCSVMHEALHNHYTQKSLSLSPGK | | |
| 607 | ASTKGPSVFPLAPSSKSTSGGTAALGCLVKDYFPEPVT | IgG Heavy | |
| | VSWNSGALTSGVHTFPAVLQSSGLYSLSSVVTVPSSSL | chain | |
| | GTQTYICNVNHKPSNTKVDKKVEPKSCDKTHTCPPCP | constant | |
| | APELLGGPSVFLFPPKPKDTLMISRTPEVTCVVVDVSHE | region, | |
| | DPEVKFNWYVDGVEVHNAKTKPREEQYNSTYRVVSV | CH1-Fc | |
| | LTVLHQDWLNGKEYKCKVSNKALPAPIEKTISKAKGQ | | |
| | PREPQVYTLPPSREEMTKNQVSLTCLVKGFYPSDIAVE | | |
| | WESNGQPENNYKTTPPVLDSDGSFFLYSKLTVDKSRW | | |
| | QQGNVFSCSVMHEALHNHYTQKSLSLSPGK | | |
| 608 | RTVAAPSVFIFPPSDEQLKSGTASVVCLLNNFYPREAK | Light | |
| | VQWKVDNALQSGNSQESVTEQDSKDSTYSLSSTLTLS | chain | |
| | KADYEKHKVYACEVTHQGLSSPVTKSFNRGEC | (kappa) | |
| | | constant | |
| | | region, CL | |
| 609 | DVVMTQTPLTLSVTIGQPASISCKSSQSLLYSNGETYLN | huG14-1- | |
| | WLLQRPGQSPKRLIYLVSKLDSGVPDRVTGSGSGTDFT | Light | |
| | LKISRVEAEDLGVYYCVQGTYFPYTFGGGTKLEIKRTV | chain | |
| | AAPSVFIFPPSDEQLKSGTASVVCLLNNFYPREAKVQW | | |
| | KVDNALQSGNSQESVTEQDSKDSTYSLSSTLTLSKADY | | |
| | EKHKVYACEVTHQGLSSPVTKSFNRGEC | | |
| 610 | EVQLVESGGGLVKPGGSLRLSCAASGLTFSNYWMNW | huG14-1- | |
| | VRQAPGKGLEWVALIRLYSDNYAIHYAESVKGRFTISR | heavy | |
| | DDSKSTVYLQMNSLKTEDTAVYYCTTGTDYWGQGTT | chain | |
| | VTVSSASTKGPSVFPLAPSSKSTSGGTAALGCLVKDYF | | |
| | PEPVTVSWNSGALTSGVHTFPAVLQSSGLYSLSSVVTV | | |
| | PSSSLGTQTYICNVNHKPSNTKVDKKVEPKSCDKTHTC | | |
| | PPCPAPELLGGPSVFLFPPKPKDTLMISRTPEVTCVVVD | | |
| | VSHEDPEVKFNWYVDGVEVHNAKTKPREEQYNSTYR | | |
| | VVSVLTVLHQDWLNGKEYKCKVSNKALPAPIEKTISK | | |
| | AKGQPREPQVYTLPPSREEMTKNQVSLTCLVKGFYPSD | | |
| | IAVEWESNGQPENNYKTTPPVLDSDGSFFLYSKLTVDK | | |
| | SRWQQGNVFSCSVMHEALHNHYTQKSLSLSPGK | | |
| 611 | DVVMTQTPLTLSVTIGQPASISCKSSQSLLYSNGETYLN | G14- | |
| | WLLQRPGQSPKRLIYLVSKLDSGVPDRVTGSGSGTDFT | Light | |
| | LKISRVEAEDLGVYYCVQGTYFPYTFGGGTKLEIKRTV | chain | |
| | AAPSVFIFPPSDEQLKSGTASVVCLLNNFYPREAKVQW | (chimeric) | |
| | KVDNALQSGNSQESVTEQDSKDSTYSLSSTLTLSKADY | | |
| | EKHKVYACEVTHQGLSSPVTKSFNRGEC | | |
| 612 | EVKLEESGGGLVQPGGSMKLSCVASGLTFSNYWMNW | G14- | |
| | VRQSPEKGLEWVALIRLYSDNYAIHYAESVKGRFTISR | Heavy | |
| | DDSKSSVYLQMNNLRAEDTGIYYCTTGTDYWGQGTT | chain | |
| | LTVSSASTKGPSVFPLAPSSKSTSGGTAALGCLVKDYFP | (chimeric) | |
| | EPVTVSWNSGALTSGVHTFPAVLQSSGLYSLSSVVTVP | | |
| | SSSLGTQTYICNVNHKPSNTKVDKKVEPKSCDKTHTCP | | |
| | PCPAPELLGGPSVFLFPPKPKDTLMISRTPEVTCVVVDV | | |
| | SHEDPEVKFNWYVDGVEVHNAKTKPREEQYNSTYRV | | |
| | VSVLTVLHQDWLNGKEYKCKVSNKALPAPIEKTISKA | | |
| | KGQPREPQVYTLPPSREEMTKNQVSLTCLVKGFYPSDI | | |
| | AVEWESNGQPENNYKTTPPVLDSDGSFFLYSKLTVDK | | |
| | SRWQQGNVFSCSVMHEALHNHYTQKSLSLSPGK | | |
| 703 | KSSHSLLYSNKRTYLN | LCDR1 | huG14-302 |
| 704 | LVSKLDS | LCDR2 | huG14-302 |
| 705 | IQGTYFPYT | LCDR3 | huG14-302 |
| 706 | NYWGN | HCDR1 | huG14-302 |
| 707 | LIRLYSDNYAIHYAESVKG | HCDR2 | huG14-302 |
| 708 | GTDY | HCDR3 | huG14-302 |
| 713 | KSSHSLLYSNKKTYLN | LCDR1 | huG14-303 |
| 714 | LVSKLDS | LCDR2 | huG14-303 |
| 715 | IQGTYFPYT | LCDR3 | huG14-303 |
| 716 | NYWGN | HCDR1 | huG14-303 |
| 717 | LIRLYSDNYAIHYAESVKG | HCDR2 | huG14-303 |
| 718 | GTDY | HCDR3 | huG14-303 |
| 723 | KSSHSLLYSNKRTYLN | LCDR1 | huG14-304 |
| 724 | LVSKLDS | LCDR2 | huG14-304 |
| 725 | IQGTYFPYT | LCDR3 | huG14-304 |
| 726 | NYWMN | HCDR1 | huG14-304 |
| 727 | LIRLYSDNYAIHYAESVKG | HCDR2 | huG14-304 |
| 728 | GTDN | HCDR3 | huG14-304 |
| 733 | KSSHSLLYSNKKTYLN | LCDR1 | huG14-305 |
| 734 | LVSKLDS | LCDR2 | huG14-305 |
| 735 | IQGTYFPYT | LCDR3 | huG14-305 |
| 736 | NYWMN | HCDR1 | huG14-305 |
| 737 | LIRLYSDNYAIHYAESVKG | HCDR2 | huG14-305 |
| 738 | GTDN | HCDR3 | huG14-305 |
| 743 | KSSHSLLYSNKKTYLN | LCDR1 | huG14-306 |
| 744 | LVSKLDS | LCDR2 | huG14-306 |
| 745 | IQGTYFPYT | LCDR3 | huG14-306 |
| 746 | NYWGN | HCDR1 | huG14-306 |
| 747 | LIRLYSDNYAIHYAESVKG | HCDR2 | huG14-306 |
| 748 | GTDN | HCDR3 | huG14-306 |

*CDRs are defined by Kabat Definition in Table 1 and in the hereinafter Examples.

### Therapeutic Methods

Described herein is a method of inhibiting CD24 signaling in a cancer cell comprising contacting the cancer cell with an antibody of the present disclosure. In certain embodiments, CD24 signaling is not inhibited in non-neoplastic B lymphocytes or non-neoplastic T lymphocytes. In certain embodiments, the cancer cell is a cancer cell is in vivo in an individual. In certain embodiments, signaling though CD24 is reduced by about 10%, 20%, 35%, 30%, 40%, 50%, 60%, 70%, 80%, 90% or more. In certain embodiments, signaling through CD24 is reduced to levels not detectable above background levels. In certain embodiments, signaling through SHP2 is reduced. In certain embodiments, signaling through SHP2 is reduced by about 10%, 20%, 35%, 30%, 40%, 50%, 60%, 70%, 80%, 90% or more. Signaling through SHP2 can be evidence by levels of phosphorylation of SHP2.

In a certain aspect described herein is method of treating cancer comprising administering an antibody of the disclosure to an individual afflicted with a cancer or a tumor. The antibodies of the disclosure may be used in a method of treating cancer. The antibodies of the disclosure may be used in the manufacture of a medicament for treating cancer. In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In certain embodiments, tumors which can be treated with the antibodies of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed.

In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 1 mg/kg and about 20 mg/kg. In certain embodiments, the therapeutically acceptable amount is between about 1 mg/kg and about 10 mg/kg. In certain embodiments, the therapeutically acceptable amount is between about 5 mg/kg and about 30 mg/kg. In certain embodiments, the therapeutically acceptable amount is between about 5 mg/kg and about 20 mg/kg. Therapeutically effective amounts include amounts sufficient to ameliorate one or more symptoms associated with the disease or affliction to be treated.

### Pharmaceutically Acceptable Excipients, Carriers, and Diluents

In certain embodiments the anti-CD24 antibodies of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration (e.g., a needle and/or syringe).

In certain embodiments, described herein is a method of preparing a cancer treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### EXAMPLES

The following illustrative examples are representative of embodiments of compositions and methods described herein and are not meant to be limiting in any way.

### Example 1—CD24 Antigen Design, Expression, and Purification

The amino acid sequences of the antigen and proteins for use in raising the antibodies of the present disclosure were obtained from UniProt (human CD24, Uniprot number: P25063). A SUMO-his tag and GST tag were fused to the CD24 protein. The gene encoding SUMO-his tag and GST tag fusion protein was cloned into a pET21b vector (Novagen, Cat No. 69741), expressed by an E. coli expression system and purified. The gene encoding the GST-tag fusion protein was cloned into a pTT5 vector, and was transiently expressed with Expi293F cells (Thermo, Cat No. A14527) and purified. CD24 antigens referred to in this section refer to human CD24 protein, unless otherwise specified.

Fusion protein of CD24 (27-56aa) extracellular domain and GST, i.e., CD24 (27-56aa)-GST (NEOX-2, SEQ ID NO: 503), for mouse immunization and detection: MSETTTGTSSNSSQSTSNSGLAPNPTNATTKSPILGYWKIKGLVQPTRLLLEYLEEKYEEHL YERDEGDKWRNKKFELGLEFPNLPYYIDGDVKLTOSMAIIRYIADKHNMLGGCPKERAEISM LEGAVLDIRYGVSRIAYSKDFETLKVDFLSKLPEMLKMFEDRLCHKTYLNGDHVTHPDFMLY DALDVVLYMDPMCLDAFPKLVCFKKRIEAIPQIDKYLKSSKYLAWPLQGWQATFGGGDHPP KSD Note: underlined portion denotes CD24 (27-56aa), italic portion denotes GST tag.

Fusion protein of CD24 (27-56aa) extracellular domain and SUMO-his, i.e., CD24 (27-56aa)-SUMO (NEOX-27, SEQ ID NO: 504), for detection MSETTTGTSSNSSOSTSNSGLAPNPTNATTKAAMSDSEVNQEAKPEVKPEVKPETHINLKV SDGSSEIFFKIKKTTPLRRLMEAFAKRQGKEMDSLRFLYDGIRIQADQTPEDLDMEDNDIIEA HREQIGGHHHHHH Note: underlined portion denotes CD24 (27-56aa), italic portion denotes SUMO-his tag.

Fusion protein of CD24 (27-56aa) extracellular domain and GST, i.e., CD24 (27-56aa)-GST (NEOX-4, SEQ ID NO: 505), for detection MGSTAILGLLLAVLQGGRASETTTGTSSNSSQSTSNSGLAPNPTNATTKSPILGYWKIKG LVQPTRLLLEYLEEKYEEHLYERDEGDKWRNKKFELGLEFPNLPYYIDGDVKLTQSMAIIRYI ADKHNMLGGCPKERAEISMLEGAVLDIRYGVSRIAYSKDFETLKVDFLSKLPEMLKMFEDRL CHKTYLNGDHVTHPDFMLYDALDVVLYMDPMCLDAFPKLVCFKKRIEAIPQIDKYLKSSKYI AWPLQGWQATFGGGDHPPKSD Note: bold portion denotes signal peptide, underlined portion denotes CD24 (27-56aa), italic portion denotes GST tag.

### Purification of CD24 (27-56Aa)-GST (NEOX-2)

Cultured samples were centrifuged (13,000 rpm, 4 °C, 20 min) to collect the bacterial cells. The bacterial cells were resuspended with an appropriate volume of Buffer A (50 mM Tris, 500 mM NaCl, 5% glycerol, 1 mM TCEP, pH 7.5), and were disrupted with a sonicator (Fisher Scientific, FB505/705) (3 s/3 s, 30 min), and then centrifuged at a high speed (13,000 rpm, 4 °C, 30 min) to collect the supernatant samples expressed by the cells. A 5 ml GST resin affinity column (Cytiva, Cat No. 17075605) was pre-equilibrated with buffer A (50 mM Tris, 500 mM NaCl, 5% glycerol, 1 mM TCEP, pH 7.5), and the supernatant samples were loaded onto the column. The column was rinsed with buffer A (50 mM Tris, 500 mM NaCl, 5% glycerol, 1 mM TCEP, pH 7.5) until no protein could be detected with Coomassie brilliant blue G-250. Then the column was eluted with Buffer F (Buffer A containing 10 mM Glutathione, RSH) and the target CD24 (27-56aa)-GST protein was collected. Finally, the CD24 (27-56aa)-GST protein was changed from Buffer F to PBS buffer by dialysis. The presence and purity of CD24 (27-56aa)-GST protein was confirmed by SDS-PAGE and HPLC.

### Purification of CD24 (27-56Aa)-SUMO his (NEOX-27)

Cultured samples were centrifuged (13,000 rpm, 4 °C, 20 min) to collect the bacterial cells. The bacterial cells were resuspended with an appropriate volume of Buffer A (50 mM Tris, 500 mM NaCl, 5% glycerol, 1 mM TCEP, pH 7.5), and were disrupted with a sonicator (Fisher Scientific, FB505/705) (3 s/3 s, 30 min), and then centrifuged at a high speed (13,000 rpm, 4 °C, 30 min) to collect the supernatant samples expressed by the cells. A 5 ml Ni Sepharose excel affinity column (Cytiva, Cat No. 17371201) was pre-equilibrated with buffer A (50 mM Tris, 500 mM NaCl, 5% glycerol, 1 mM TCEP, pH 7.5), and the supernatant samples were loaded onto the column. The column was rinsed with buffer A (50 mM Tris, 500 mM NaCl, 5% glycerol, 1 mM TCEP, pH 7.5) until no protein could be detected with Coomassie brilliant blue G-250. The column was rinsed with Buffer B (Buffer A containing 20 mM imidazole) and Buffer C (Buffer A containing 50 mM imidazole) sequentially, and then eluted with Buffer E (Buffer A containing 250 mM imidazole) and the target CD24 (27-56aa)-SUMO his protein was collected. Finally, the CD24 (27-56aa)-SUMO his protein was changed from Buffer E to PBS buffer by dialysis. The presence and purity of the CD24 (27-56aa)-SUMO his protein was confirmed by SDS-PAGE and HPLC.

### Purification of CD24 (27-56Aa)-GST (NEOX-4)

Cultured samples were centrifuged at a high speed (13,000 rpm, 4 °C, 30 min) to collect the culture supernatant. A 1 ml GST resin affinity column (Cytiva, Cat No. 17075605) was pre-equilibrated with buffer A (50 mM Tris, 500 mM NaCl, 5% glycerol, 1 mM TCEP, pH 7.5), and the supernatant samples were loaded onto the column. The column was rinsed with buffer A (50 mM Tris, 500 mM NaCl, 5% glycerol, 1 mM TCEP, pH 7.5) until no protein could be detected with Coomassie brilliant blue G-250. Then the column was eluted with Buffer F (Buffer A containing 10 mM Glutathione, RSH) and the target CD24 (27-56aa)-GST protein was collected. Finally, the CD24 (27-56aa)-GST protein was changed from Buffer F to PBS buffer by dialysis. The presence and purity of the CD24 (27-56aa)-GST protein was confirmed by SDS-PAGE and HPLC.

### Purification of Recombinant Antibodies

Supernatant samples expressed by the cells were centrifuged at high speed (13,000 rpm, 4 °C, 30 min) to remove impurities, and the expressed supernatants of recombinant antibodies were purified with a Protein A column. The target protein was eluted with 100 mM acetic acid pH 3.0 and neutralized with 1M Tris-HCl, pH 8.0. The eluted samples were concentrated and further purified with a PBS-equilibrated gel chromatography column Superdex200 (GE), and the depolymerized peaks were collected and aliquoted for later use.

### Example 2—Generation of Anti CD24 Antibodies

### Immunization

Anti-human CD24 monoclonal antibodies were produced by immunizing mice. SJL white mice, female, 6-8 weeks old (Shanghai Slac Laboratory Animal Co. Ltd., Animal production license number: SCXK (Shanghai) 2017-0005) were used in the experiment. The breeding environment was SPF grade. After the mice were purchased, they were raised in the laboratory environment for 1 week, with a 12/12 hours light/dark cycle adjustment, at a temperature of 20-25 °C and a humidity of 40-60%. The mice having adapted to the environment were immunized in accordance with the following protocol. The immunizing antigen was a GST-tagged human CD24 extracellular domain fusion protein (CD24 (27-56aa)-GST (NEOX-2)). The mice were immunized with CFA/IFA/Alum/CpG. The ratio of antigen to adjuvant was 1:1, and the antigen and adjuvant were inoculated after being mixed quickly and thoroughly. First immunization: 50 μg antigen/mice/time; booster immunization: 25 μg antigen/mouse/time. The inoculation interval was 7 days, and a total of 5 immunizations were performed. Starting from the third immunization, blood was collected 7 days after each immunization, and the antibody titer in the mouse serum was determined by ELISA method. Mice with high antibody titers in the serum and titers tended to plateau were subjected to splenocyte fusion. Three days before the splenocyte fusion, the booster immunization was performed by intraperitoneal (IP) injection of antigen solution prepared in normal saline at a dose of 50 μg/mouse.

### Hybridoma Formation

Mouse spleen lymphocytes were mixed with Sp2/0 myeloma cells (CRL-8287TM) at a ratio of 1:1-10:1 using a TBX electroporator, and the hybridoma cells were obtained by a round of electric shock (60V, 30 s; pulse 40 ms, 1700V). The fused hybridoma cells were resuspended in complete medium (containing 20% FBS, 1×HAT, 1×DMEM medium) at a density of 4-5E5/ml, and were seeded at 200 μl/well in a 96-well plate and cultured at 37 °C, 5% CO₂ for 7 days. After culturing for 8 days, the culture medium was switched to HT complete medium (containing 20% FBS, 1×HT and 1×DMEM medium) at 200 μl/well, and the cells were cultured at 37 °C, 5% CO₂ for 2 days, then subjected to ELISA detection.

### Screening of Hybridoma Cells

ELISA and FACS (antigen being detected: CD24 (27-56aa)-SUMO his (NEOX-27)) were used to screen for positive hybridomas. Clones positive by ELISA and FACS were expanded, and subjected to 2 to 3 rounds of subclone screening until single cell clones were obtained. The hybridoma monoclonal G14 was obtained through the above experimental screening.

### Cloning and Sequence Determination of Positive Hybridoma Clones

Hybridoma cells in logarithmic growth phase were collected, and RNA was extracted with Trizol (Invitrogen, Cat No. 15596-018) according to the kit instructions and reverse transcribed with PrimeScript™ Reverse Transcriptase Kit (Takara, Cat No. 2680A). The cDNA obtained by reverse-transcription was PCR-amplified with a mouse Ig-Primer Set (Novagen, TB326 Rev.B 0503) to amplify the variable region sequences of the antibody heavy and light chains, cloned into a vector, and then subjected to sequencing. The amino acid sequences corresponding to the DNA sequences of the heavy and light chain variable regions of murine monoclonal antibody G14 were obtained as:

G14 heavy chain variable region
(G14VH, SEQ ID NO: 2)
EVKLEESGGGLVQPGGSMKLSCVASGLTFSNYWMNWVRQSPEKGLEWVALIRLYSDN
YAIHYAESVKGRFTISRDDSKSSVYLQMNNLRAEDTGIYYCTTGTDYWGQGTTLTVSS
G14 light chain variable region
(G14VL, SEQ ID NO: 1)
DVVMTQTPLTLSVTIGQPASISCKSSQSLLYSNGETYLNWLLQRPGQSPKRLIYLVSKLD
SGVPDRVTGSGSGTDFTLKISRVEAEDLGVYYCVQGTYFPYTFGGGTKLEIK

### Example 3—Humanization of Anti-Human CD24 Murine Monoclonal Antibodies

By aligning the germline gene database of IMGT human antibody heavy and light chain variable regions and MOE software, the germline genes of heavy chain and light chain variable regions with high homology to the murine monoclonal antibody G14 antibody were selected as templates respectively, so as to grating the CDRs of the murine antibodies into the corresponding humanization templates to form humanized variable region sequences with the order of FR1-CDR1-FR2-CDR2-FR3-CDR3-FR4.

### 1.1 G14 Humanization Framework Grafting—Design 1

With the light chain variable region sequence of the murine antibody G14 remained unchanged (murine), the heavy chain variable region was humanized using germline IGHV3-15*01 IGHJ6*019 as template. The humanized variable region sequence combination huG14-1_graft was as follows:

G14VL
DVVMTQTPLTLSVTIGQPASISCKSSQSLLYSNGETYLNWLLQRPGQSPKRLIYLVSKLD
SGVPDRVTGSGSGTDFTLKISRVEAEDLGVYYCVQGTYFPYTFGGGTKLEIK
G14VH_Graft
EVQLVESGGGLVKPGGSLRLSCAASGLTFSNYWMNWVRQAPGKGLEWVGLIRLYSDN
YAIHYAESVKGRFTISRDDSKNTLYLQMNSLKTEDTAVYYCTTGTDYWGQGTTVTVSS

underlined portion denotes CDR sequence.

### 1.2 Mutations for G14 Humanization Framework Grafting—Design 1

Three mutations (G49A, N79S, L81V) were introduced into the FR region of the G14VH_Graft sequence to obtain the heavy chain variable region sequence huG14VH, which was paired with the light chain variable region sequence G14VL to obtain the antibody huG14-1, and its VH/VL combination is shown as follows:

G14VL
(SEQ ID NO: 101)
DVVMTQTPLTLSVTIGQPASISCKSSQSLLYSNGETYLNWLLQRPGQSPKRLIYLVSKLD
SGVPDRVTGSGSGTDFTLKISRVEAEDLGVYYCVQGTYFPYTFGGGTKLEIK
huG14VH
(SEQ ID NO: 102)
EVQLVESGGGLVKPGGSLRLSCAASGLTFSNYWMNWVRQAPGKGLEWVALIRLYSDN
YAIHYAESVKGRFTISRDDSKSTVYLQMNSLKTEDTAVYYCTTGTDYWGQGTTVTVSS

underlined portion denotes CDR sequence.

### 2.1 G14 Humanization Framework Grafting-Design 2

For G14 humanization framework grafting-design 2, the light chain variable region was humanized using the germline IGKV2-28*01 IGKJ2*01 as template, and the heavy chain variable region was humanized using the germline IGHV3-15*01 IGHJ6*019 as template. The grafted variable region sequence combination huG14-2_graft was as obtained as follows:

G14VL_Graft1:
DIVMTQSPLSLPVTPGEPASISCKSSQSLLYSNGETYLNWYLQKPGQSPQLLIYLVSK
LDSGVPDRFSGSGSGTDFTLKISRVEAEDLGVYYCVQGTYFPYTFGQGTKLEIK
G14VH_Graft:
EVQLVESGGGLVKPGGSLRLSCAASGLTFSNYWMNWVRQAPGKGLEWVGLIRLYSDN
YAIHYAESVKGRFTISRDDSKNTLYLQMNSLKTEDTAVYYCTTGTDYWGQGTTVTVSS

underlined portion denoting CDR sequence.

### 2.2 Mutations for G14 Humanization Framework Grafting-Design 2

Multiple residues sites in the FR region of the G14VL_Graft1 sequence were mutated (sites selected from: 12, Y41, L51, E17, P12, Q105), so that the following light chain variable region sequences were obtained: huG14VL1 (12V, Y41L, L51R), huG14VL1-1 (12V, Y41L, L51R, E17R), huG14VL1-2 (12V, Y41L, L51R, P12Y), huG14VL1-3 (12V, Y41L, L51R, Q1051), huG14VL1-15 (12V, Y41L, L51R, P12Y, E17R), huG14VL1-16 (12V, Y41L, L51R, S10R, P12Y, E17R, Q1051). In addition, three mutations (G49A, N79S, L81V) were introduced into the FR region of the G14VH_Graft sequence to obtain the heavy chain variable region sequence huG14VH. The resulting pairs of humanized VH/VL combinations were as follows:

Humanized variable region sequence combination huG14-2:
huG14VL1
(SEQ ID NO: 201)
DVVMTQSPLSLPVTPGEPASISCKSSQSLLYSNGETYLNWLLQKPGQSPQRLIYLVSKLD
SGVPDRFSGSGSGTDFTLKISRVEAEDLGVYYCVQGTYFPYTFGQGTKLEIK
huG14VH (SEQ ID NO: 202)
EVQLVESGGGLVKPGGSLRLSCAASGLTFSNYWMNWVRQAPGKGLEWVALIRLYSDN
YAIHYAESVKGRFTISRDDSKSTVYLQMNSLKTEDTAVYYCTTGTDYWGQGTTVTVSS
Humanized variable region sequence combination huG14-201:
huG14VL1-1 (SEQ ID NO: 209):
DVVMTQSPLSLPVTPGRPASISCKSSQSLLYSNGETYLNWLLQKPGQSPQRLIYLVSKLD
SGVPDRFSGSGSGTDFTLKISRVEAEDLGVYYCVQGTYFPYTFGQGTKLEIK
huG14VH (SEQ ID NO: 210)
EVQLVESGGGLVKPGGSLRLSCAASGLTFSNYWMNWVRQAPGKGLEWVALIRLYSDN
YAIHYAESVKGRFTISRDDSKSTVYLQMNSLKTEDTAVYYCTTGTDYWGQGTTVTVSS
Humanized variable region sequence combination huG14-202:
huG14VL1-2 (SEQ ID NO: 211):
DVVMTQSPLSLYVTPGEPASISCKSSQSLLYSNGETYLNWLLQKPGQSPQRLIYLVSKLD
SGVPDRFSGSGSGTDFTLKISRVEAEDLGVYYCVQGTYFPYTFGQGTKLEIK
huG14VH (SEQ ID NO: 212):
EVQLVESGGGLVKPGGSLRLSCAASGLTFSNYWMNWVRQAPGKGLEWVALIRLYSDN
YAIHYAESVKGRFTISRDDSKSTVYLQMNSLKTEDTAVYYCTTGTDYWGQGTTVTVSS
Humanized variable region sequence combination huG14-203:
huG14VL1-3 (SEQ ID NO: 213):
DVVMTQSPLSLPVTPGEPASISCKSSQSLLYSNGETYLNWLLQKPGQSPQRLIYLVSKLD
SGVPDRFSGSGSGTDFTLKISRVEAEDLGVYYCVQGTYFPYTFGIGTKLEIK
huG14VH (SEQ ID NO: 214):
EVQLVESGGGLVKPGGSLRLSCAASGLTFSNYWMNWVRQAPGKGLEWVALIRLYSDN
YAIHYAESVKGRFTISRDDSKSTVYLQMNSLKTEDTAVYYCTTGTDYWGQGTTVTVSS
Humanized variable region sequence combination huG14-2011:
huG14VL1-15 (SEQ ID NO: 279):
DVVMTQSPLSLYVTPGRPASISCKSSQSLLYSNGETYLNWLLQKPGQSPQRLIYLVSKLD
SGVPDRFSGSGSGTDFTLKISRVEAEDLGVYYCVQGTYFPYTFGQGTKLEIK
huG14VH (SEQ ID NO: 280):
EVQLVESGGGLVKPGGSLRLSCAASGLTFSNYWMNWVRQAPGKGLEWVALIRLYSDN
YAIHYAESVKGRFTISRDDSKSTVYLQMNSLKTEDTAVYYCTTGTDYWGQGTTVTVSS
Humanized variable region sequence combination huG14-2012:
huG14VL1-16 (SEQ ID NO: 281):
DVVMTQSPLRLYVTPGRPASISCKSSQSLLYSNGETYLNWLLQKPGQSPQRLIYLVSKL
DSGVPDRFSGSGSGTDFTLKISRVEAEDLGVYYCVQGTYFPYTFGIGTKLEIK
huG14VH (SEQ ID NO: 282):
EVQLVESGGGLVKPGGSLRLSCAASGLTFSNYWMNWVRQAPGKGLEWVALIRLYSDN
YAIHYAESVKGRFTISRDDSKSTVYLQMNSLKTEDTAVYYCTTGTDYWGQGTTVTVSS

### 3.1 G14 Humanization Framework Grafting-Design 3

For G14 humanization framework grafting-design 3, the light chain variable region was humanized using the germline GKV2-30*01 IGKJ2*01 as template, and the heavy chain variable region was humanized using the germline IGHV3-15*01 IGHJ6*019 as template. The humanized variable region sequence combination huG14-3_graft was obtained as follows:

G14VL_Graft2:
DVVMTQSPLSLPVTLGQPASISCKSSQSLLYSNGETYLNWFQQRPGQSPRRLIYLVSKLD
SGVPDRFSGSGSGTDFTLKISRVEAEDVGVYYCVQGTYFPYTFGQGTKLEIK
G14VH_Graft:
EVQLVESGGGLVKPGGSLRLSCAASGLTFSNYWMNWVRQAPGKGLEWVGLIRLYSDN
YAIHYAESVKGRFTISRDDSKNTLYLQMNSLKTEDTAVYYCTTGTDYWGQGTTVTVSS

### 3.2 Mutations for G14 Humanization Framework Grafting-Design 3

Two mutations (F41L, V88L) were introduced into the FR region of the G14VL_Graft2 sequence to obtain the humanized light chain variable region sequence huG14VL2. Three mutations (G49A, N79S, L81V) were introduced into the FR region of the G14VH_Graft sequence to obtain the humanized heavy chain variable region sequence huG14VH. The humanized antibody huG14-3 was obtained, and its VH/VL is expressed as follows:

huG14VL2 (SEQ ID NO: 301):
DVVMTQSPLSLPVTLGQPASISCKSSQSLLYSNGETYLNWLQQRPGQSPRRLIYLVSKLD
SGVPDRFSGSGSGTDFTLKISRVEAEDLGVYYCVQGTYFPYTFGQGTKLEIK
huG14VH (SEQ ID NO: 302):
EVQLVESGGGLVKPGGSLRLSCAASGLTFSNYWMNWVRQAPGKGLEWVALIRLYSDN
VYAIHYAESVKGRFTISRDDSKSTVYLQMNSLKTEDTAVYYCTTGTDYWGQGTTVTVSS

For the purpose of illustration, all of the references to the mutation sites in the FR region in Example 3 adopt a sequential numbering method, and the comparison table between the numbering method and the Kabat numbering is shown in the following table:

For humanization framework-design 2:

| VL | | VH | |
|---|---|---|---|
| IGKV2-28*01 IGKJ2*01 | | IGHV3-15*01 IGHJ6*019 | |
| in sequence | in Kabat | in sequence | in Kabat |
| order | system | order | system |
| I2 | I2 | G49 | G49 |
| Y41 | Y36 | N79 | N76 |
| L51 | L46 | L81 | L78 |
| E17 | E17 | | |
| S10 | S10 | | |
| P12 | P12 | | |
| Q105 | Q100 | | |

For humanization framework-design 3:

| VL | | VH | |
|---|---|---|---|
| IGKV2-30*01 IGKJ2*01 | | IGHV3-15*01 IGHJ6*019 | |
| in sequence | in Kabat | in sequence | in Kabat |
| order | system | order | system |
| F41 | F36 | G49 | G49 |
| L88 | L83 | N79 | N76 |
| | | L81 | L78 |

### Example 4—Affinity Maturation of Anti-Human CD24 Humanized Antibodies

Single-point mutations and multi-point mutations to the CDRs of the VH and VL in the variable regions described above were performed, and these mutant antibody proteins were expressed from CHO cells. The EC50 values and/or affinities of the mutants and CD24 protein were determined by ELISA and/or SPR methods. Affinity matured antibodies were thus obtained.

1. Affinity Maturation Based on Humanization Design 1 (huG14-1): Single Mutation Site Design and Multiple Mutation Combination Design for CDR

On the basis of the humanized sequences of antibody huG14-1, single-point mutation and multiple-point mutation were introduced into the CDR regions of the heavy chain variable region VH and the light chain variable region VL. The resulting final VH and VL sequences are shown in the following table:

| VL | | VH | |
|---|---|---|---|
| G14VL1 | G14VL + Q27F | huG14VH1 | huG14VH + M34G |
| G14VL2 | G14VL + Q27H | huG14VH2 | huG14VH+ Y104N |
| G14VL3 | G14VL + V94I | huG14VH3 | huG14VH + Y104F |
| G14VL4 | G14VL + E35I | huG14VH4 | huG14VH + M34G/ |
| | | | Y104N |
| G14VL5 | G14VL + E35K | | |
| G14VL6 | G14VL + E35R | | |
| G14VL7 | G14VL + E35V | | |
| G14VL8 | G14VL + G34W | | |
| G14VL9 | G14VL + Q27H/ | | |
| | V94I | | |
| G14VL10 | G14VL + Q27H/ | | |
| | E35K | | |
| G14VL11 | G14VL + Q27H/ | | |
| | E35R | | |
| G14VL12 | G14VL + E35K/ | | |
| | V94I | | |
| G14VL13 | G14VL + E35R/ | | |
| | V94I | | |
| G14VL14 | G14VL + Q27H | | |
| | /E35K/V94I | | |
| G14VL15 | G14VL + Q27H/ | | |
| | E35R/V94I | | |

The above VH and VL sequences were paired to obtain multiple heavy chain/light chain variable region sequence combinations (i.e., affinity matured antibodies), expressed as follows:

| | huG14VH | huG14VH1 | huG14VH2 | huG14VH3 | huG14VH4 |
|---|---|---|---|---|---|
| G14VL | huG14-1 | huG14-109 | huG14-110 | huG14-111 | |
| G14VL1 | huG14-101 | | | | |
| G14VL2 | huG14-102 | | | | |
| G14VL3 | huG14-103 | | | | |
| G14VL4 | huG14-104 | | | | |
| G14VL5 | huG14-105 | | | | |
| G14VL6 | huG14-106 | | | | |
| G14VL7 | huG14-107 | | | | |
| G14VL8 | huG14-108 | | | | |
| G14VL9 | huG14-112 | huG14-119 | huG14-126 | | huG14-133 |
| G14VL10 | huG14-113 | huG14-120 | huG14-127 | | huG14-134 |
| G14VL11 | huG14-114 | huG14-121 | huG14-128 | | huG14-135 |
| G14VL12 | huG14-115 | huG14-122 | huG14-129 | | huG14-136 |
| G14VL13 | huG14-116 | huG14-123 | huG14-130 | | huG14-137 |
| G14VL14 | huG14-117 | huG14-124 | huG14-131 | | huG14-138 |
| G14VL15 | huG14-118 | huG14-125 | huG14-132 | | huG14-139 |

2. Affinity Maturation Based on Humanization Design 2 (huG14-2, Etc.): CDR Multiple Mutation Combination Design

On the basis of the humanized sequences of design 2, single-point mutation and multiple-point mutation were introduced into the CDR regions of the heavy chain variable region VH and the light chain variable region VL. The resulting final VH and VL sequences are shown in the following table:

| VL | | VH | |
|---|---|---|---|
| huG14VL1-4 | huG14VL1-1 + Q27H/V94I | huG14VH1 | huG14VH + M34G |
| huG14VL1-5 | huG14VL1-1 + Q27H/E35K | huG14VH2 | huG14VH + Y104N |
| huG14VL1-6 | huG14VL1-1 + Q27H/E35R | huG14VH4 | huG14VH + M34G/Y104N |
| huG14VL1-7 | huG14VL1-1 + E35K/V94I | | |
| huG14VL1-8 | huG14VL1-1 + E35RV94I | | |
| huG14VL1-9 | huG14VL1-1 + Q27H/E35K/V94I | | |
| huG14VL1-10 | huG14VL1-1 + Q27H/E35R/V94I | | |
| huG14VL1-11 | huG14VL1-15 + Q27H/E35R/V94I | | |
| huG14VL1-12 | huG14VL1-16 + Q27H/E35R/V94I | | |
| huG14VL1-13 | huG14VL1-1 + | | |
| | Q27H/G34K/E35K/V94I | | |
| huG14VL1-14 | huG14VL1-15 + | | |
| | Q27H/G34K/E35R/V94I | | |

The above VH and VL sequences were paired to obtain multiple heavy chain/light chain variable region sequence combinations (i.e., affinity matured antibodies), expressed as follows:

| | huG14VH | huG14VH1 | huG14VH2 | huG14VH4 |
|---|---|---|---|---|
| huG14VL1-4 | huG14-204 | huG14-211 | huG14-218 | huG14-225 |
| huG14VL1-5 | huG14-205 | huG14-212 | huG14-219 | huG14-226 |
| huG14VL1-6 | huG14-206 | huG14-213 | huG14-220 | huG14-227 |
| huG14VL1-7 | huG14-207 | huG14-214 | huG14-221 | huG14-228 |
| huG14VL1-8 | huG14-208 | huG14-215 | huG14-222 | huG14-229 |
| huG14VL1-9 | huG14-209 | huG14-216 | huG14-223 | huG14-230 |
| huG14VL1-10 | huG14-210 | huG14-217 | huG14-224 | huG14-231 |
| huG14VL1-11 | | | | huG14-232 |
| huG14VL1-12 | | | | huG14-233 |
| huG14VL1-13 | huG14-234 | | | |
| huG14VL1-14 | huG14-235 | | | |

3. Affinity Maturation Based on Humanization Design 3 (huG14-3): CDR Multiple Mutation Combination Design

On the basis of the humanized sequences of antibody huG14-3, single-point mutation and multiple-point mutation were performed on the CDR regions of the heavy chain variable region VH and the light chain variable region VL.

| VL | | VH | |
|---|---|---|---|
| huG14VL2-1 | HuG14VL2 + | huG14VH1 | huG14VH + M34G |
| | Q27H/V94I/E35R/G34K | | |
| huG14VL2-2 | HuG14VL2 + | huG14VH2 | huG14VH + Y104N |
| | Q27H/V94I/E35K/G34K | | |
| | | huG14VH4 | huG14VH + M34G/Y104N |

The above VH and VL sequences were combined to obtain multiple heavy chain/light chain variable region sequence combinations (i.e., affinity matured antibodies), expressed as follows:

| | huG14VH1 | huG14VH2 | huG14VH4 |
|---|---|---|---|
| huG14VL2-1 | huG14-302 | huG14-304 | huG14-301 |
| huG14VL2-2 | huG14-303 | huG14-305 | huG14-306 |

### Example 5—Binding and Affinity of Antibodies with Mutations Introduced to Remove Deamidation Sites were Maintained or Higher

Deamidation is a common chemical modification in antibodies that may potentially affect stability in solution. Deamidation, oxidation, or isomerization of some amino acids in the CDR region of the described antibodies were reduced by introducing mutations that would prevent such undesirable reactions. An amino acid sequence of “NG” is prone to deamidation, and the above antibodies possess an NG at positions 33-34. Thus, mutations to the CDR1 of the CD24 antibody light chain KSSQSLLYSX1X2ETYLN, wherein X1 and X2 are the amino acid residues at positions 33-34 in the variable region of the CD24 antibody light chain, respectively; X1 can be selected from Ser, Tyr or Gln, X2 can be selected from His, Lys, Arg, Trp or Ala, were constructed.

Taking the light chain G14VL14 sequence of huG14-124 variable region as an exemplary sequence, the sequences including CDR1 (KSSQSLLYSX1X2ETYLN) X1 or X2 mutation sites are shown in the table below (G14VL16 (G34H), G14VL17 (G34K), G14VL18 (G34R), G14VL19 (G34W), G14VL20 (G34Y), G14VL21 (G34A), G14VL22 (N33Y), G14VL23 (N33Q) and G14VL24 (N33S)). These single point mutation sequences were paired with the heavy chain variable region huG14VH1 sequence to generate 9 CD24 antibodies (using the same constant region as in Example 6) as shown in the table below.

TABLE
Mutation site design

| | huG14VH1 |
|---|---|
| G14VL16 | huG14-124-1 |
| (G34H) | |
| G14VL17 | huG14-124-2 |
| (G34K) | |
| G14VL18 | huG14-124-3 |
| (G34R) | |
| G14VL19 | huG14-124-4 |
| (G34W) | |
| G14VL20 | huG14-124-5 |
| (G34Y) | |
| G14VL21 | huG14-124-6 |
| (G34A) | |
| G14VL22 | huG14-124-7 |
| (N33Y) | |
| G14VL23 | huG14-124-8 |
| (N33Q) | |
| G14VL24 | huG14-124-9 |
| (N33S) | |

The affinity values of these mutant antibodies to biotinylated CD24 protein (ACRO, Cat No. CD4-H82E9) were determined by the experimental method of the Biacore affinity determination in Example 9, and the results were as follows:

TABLE
Affinity of mutant antibodies to CD24 protein

| | ka (1/Ms) | kd (1/s) | KD (M) |
|---|---|---|---|
| huG14-124 | 3.65E+05 | 5.02E−02 | 1.90E−08 |
| huG14-124-1 | 1.61E+06 | 3.35E−03 | 2.08E−09 |
| huG14-124-2 | 2.82E+06 | 4.47E−03 | 1.58E−09 |
| huG14-124-3 | 2.70E+06 | 4.11E−03 | 1.52E−09 |
| huG14-124-5 | 1.50E+06 | 3.20E−03 | 2.13E−09 |
| huG14-124-6 | 1.31E+06 | 3.33E−03 | 2.54E−09 |
| huG14-124-7 | 1.88E+07 | 2.01E−02 | 1.07E−09 |
| huG14-124-8 | 1.41E+06 | 1.50E−02 | 1.06E−08 |
| huG14-124-9 | 1.37E+06 | 1.34E−02 | 9.77E−09 |

The results showed that the affinities (KD) of the above mutant antibodies were higher than that of the huG14-124 antibody. It demonstrated that the mutations of X1 and X2 sites in the light chain CDR1 (KSSQSLLYSX1X2ETYLN) to X1 (Ser, Tyr or Gln) and X2 (His, Lys, Arg, Trp or Ala) did not decrease the affinity.

### Example 6—Preparation of Chimeric Antibodies, Humanized Antibodies and Affinity Matured Antibodies

The above heavy chain variable regions can be recombinantly expressed with the human heavy chain IgG1 constant region shown in SEQ ID NO:607 to obtain the full-length heavy chain sequence. The above light chain variable regions can be recombinantly expressed with the kappa light chain constant region sequence shown in SEQ ID NO:608 to obtain the full-length light chain sequences. The above heavy and light chain variable regions can also be recombined with other heavy and light chain constant regions of the IgG family or constant regions of mutated IgG family commonly known in the art, forming the complete heavy and light chain sequences of the antibodies. Exemplary heavy and light chain constant regions employed in the Examples of the present invention are as follows:

Sequence of heavy chain constant region
(CH1-Fc, SEQ ID NO: 607)
ASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
Sequence of light chain constant region
(CL, SEQ ID NO: 608)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

### Molecular Cloning of Recombinant Chimeric Antibody G14

The positive antibody molecules obtained by hybridoma screening were sequenced to obtain the gene sequence encoding the variable region. The head and tail primers were designed based on the sequences obtained by the sequencing, and the sequenced genes were used as the templates to construct the VH/VK gene fragments of the G14 antibody by PCR. The VH/VK gene fragments were then subjected to homologous recombination with the expression vector pcDNA3.4 (Thermo Fisher, Cat No. V001453) (with a signal peptide and hlgG1/kappa constant region gene (CH1-Fc/CL) fragment) to construct a recombinant chimeric antibody full-length expression plasmid VH-CH1-Fc-pcDNA3.4/VL-CL-pcDNA3.4, forming a G14 chimeric antibody expression construct.

The full-length amino acid sequence of the chimeric antibody (G14) is as follows:

G14 full-length Light Chain (SEQ ID NO: 611):
DVVMTQTPLTLSVTIGQPASISCKSSQSLLYSNGETYLNWLLQRPGQSPKRLIYLVSKLDSGVP
DRVTGSGSGTDFTLKISRVEAEDLGVYYCVQGTYFPYTFGGGTKLEIKRTVAAPSVFIFPPSD
EQLKSGTASVVCLLNNFYPREAKVQWKVDNALQSGNSQESVTEQDSKDSTYSLSSTLT
LSKADYEKHKVYACEVTHQGLSSPVTKSFNRGEC
G14 full-length Heavy Chain (SEQ ID NO: 612):
EVKLEESGGGLVQPGGSMKLSCVASGLTFSNYWMNWVRQSPEKGLEWVALIRLYSDNYAIHY
AESVKGRFTISRDDSKSSVYLQMNNLRAEDTGIYYCTTGTDYWGQGTTLTVSSASTKGPSVFP
LAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQ
GNVFSCSVMHEALHNHYTQKSLSLSPGK;
Note: italic + underlined portion in the sequence
denote variable region sequences (SEQ ID NO: 1 and 2,
respectively), and the rest are constant
region sequences.

### 2: Molecular Cloning of Humanized Antibodies and Affinity Matured Antibodies

The antibody sequences of humanized antibodies and affinity matured antibodies were codon-optimized to generate human codon-preferred coding gene sequences. Primers were designed to construct VH/VK gene fragments of each of the antibodies (such as huG14-1~3, and huG14-101~139, 201~235, 301~306, etc.) via PCR, and then subjected to homologous recombination with the expression vector pcDNA3.4 (Thermo Fisher, Cat No. V001453) (with signal peptide and hlgG1/kappa constant region gene (CH1-Fc/CL) fragment) to construct the recombinant humanized antibody full-length expression plasmid VH-CH1-Fc-pcDNA3.4/VL-CL-pcDNA3.4.

The full-length amino acid sequences of an exemplary humanized antibody (huG14-1) are as follows (and so on for other humanized/affinity matured antibodies):

huG14-1 full-length Light Chain (SEQ ID NO: 609):
DVVMTQTPLTLSVTIGQPASISCKSSQSLLYSNGETYLNWLLQRPGQSPKRLIYLVSKLDSGVP
DRVTGSGSGTDFTLKISRVEAEDLGVYYCVQGTYFPYTFGGGTKLEIKRTVAAPSVFIFPPSD
EQLKSGTASVVCLLNNFYPREAKVQWKVDNALQSGNSQESVTEQDSKDSTYSLSSTLT
LSKADYEKHKVYACEVTHQGLSSPVTKSFNRGEC
huG14-1 full-length Heavy Chain (SEQ ID NO: 610):
EVQLVESGGGLVKPGGSLRLSCAASGLTFSNYWMNWVRQAPGKGLEWVALIRLYSDNYAIHY
AESVKGRFTISRDDSKSTVYLQMNSLKTEDTAVYYCTTGTDYWGQGTTVTVSSASTKGPSVFP
LAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQ
GNVFSCSVMHEALHNHYTQKSLSLSPGK;
Note: italic portion + underlined portion in the
sequence denote variable region sequences
(SEQ ID NO: 101 and 102, respectively), and the
rest are constant region sequences.

### Expression and Purification of Chimeric Antibodies, Humanized Antibodies and Affinity Matured Antibodies

The plasmid expressing antibody light/heavy chains was transfected into CHO cells at a ratio of 1.5:1. After 4-6 days, the expression supernatant was collected, centrifuged at high speed (13,000 rpm, 4 °C, 30 min) to remove impurities, and purified with a Protein A column. The target antibody protein was eluted with a glycine eluent at pH3.0-pH3.5, neutralized with 1M Tris-HCl at pH8.0-9.0. The target antibody protein was replaced with PBS buffer by dialysis, aliquoted, and frozen for later use. If the antibody purity was less than 85%, the antibody was further purified by gel chromatography Superdex200 (GE) equilibrated with PBS to remove aggregates, and monomer peaks would be collected, aliquoted, and frozen for later use.

### Example 7. Determination of the Binding of Chimeric Antibodies, Humanized Antibodies and Affinity Matured Antibodies to CD24 Protein

An ELISA method was used to determine the binding capacity between CD24 protein and chimeric antibody (G14), humanized antibody (huG14-1, whose VH/VL sequences are shown in SEQ ID NO: 102 and SEQ ID NO: 101, respectively) and affinity matured antibodies (respectively labeled as huG14-101 to 139, huG14-204 to 233, and their VH/VL combinations are elaborated in the table in Example 4, and the constant regions are the same as above. Th4e ELISA was performed as follows: 1) coating of CD24 protein: CD24 (27-56aa)-GST (NEOX-2) or CD24 (27-56aa)-GST (NEOX-4) was diluted with PBS to 1 μg/ml, added to the ELISA plate with 100 μl per well, and coated overnight at 4 °C. 2) blocking: the plate was washed with 0.05% PBST solution for 3 times, and 200 μl of a 5% nonfat dry milk PBS solution which was pre-prepared in advance was added to each well of ELISA detection plate, and incubated for 2 hours at room temperature; 3) dilution and sample loading of primary antibody: the plate was washed 3 times with PBS. The CD24 antibody was diluted to 200 μg/ml, then subjected to 4-fold gradient dilution, added to the ELISA detection plate at 100 μl/well, and incubated at 37 °C for 1 hour. 4) Addition of secondary antibody: the plate was washed with PBS for 3 times. A HRP-labeled anti-human IgG Fc antibody (Jackson, Cat No. 109-035-098) was diluted for 5000 times, added to each well at 100 μl, and incubated at 37 °C for 1 h. 5) color development: the plate was washed with PBS for 6 times. 50 μl of TMB (Beijing Zhizhou Biotechnology Co., Ltd., Cat No. ZZ180001) was added to each well, placed in the dark at room temperature for 10 minutes, then 50 μl of 2M H₂SO₄ was added to terminate the color development reaction. 5) data collection and analysis: the OD450 absorbance values were read with a microplate reader (Thermo, Multiskan FC), and the EC50 values were calculated.

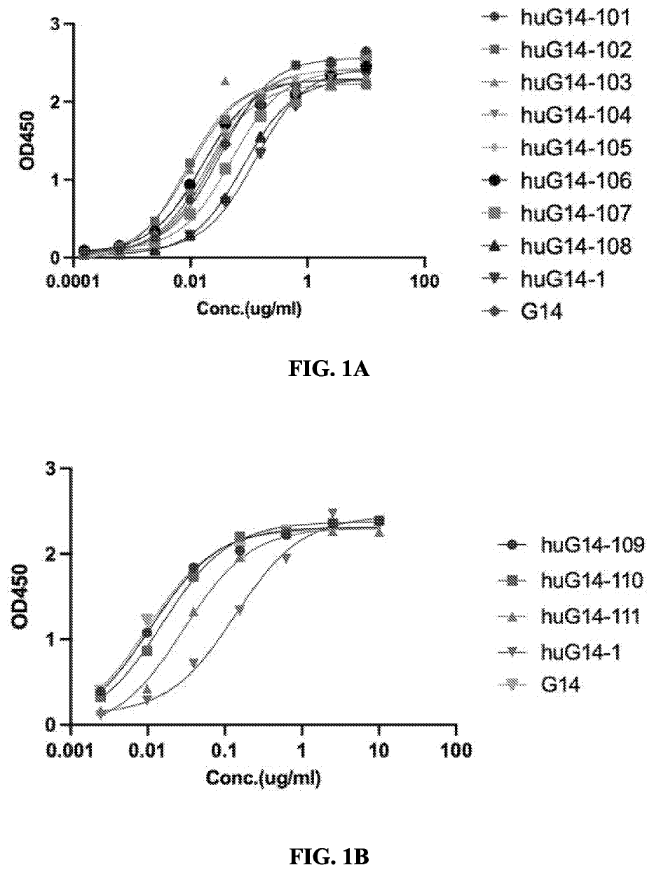

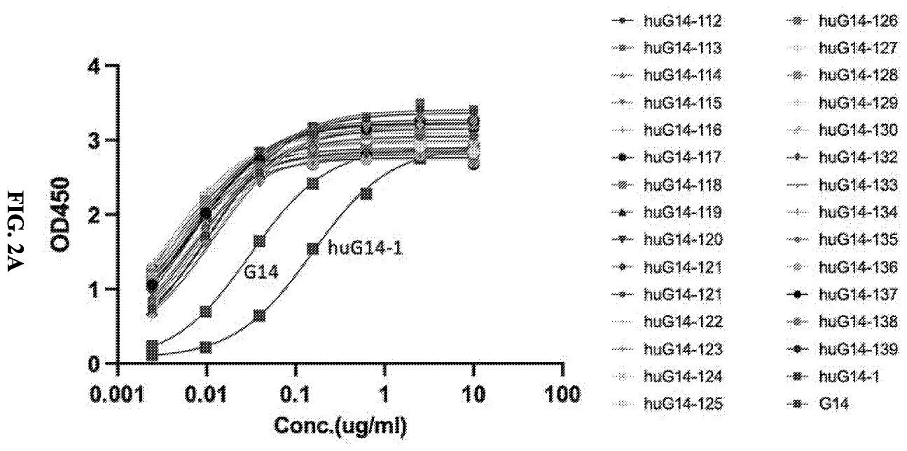

The results as shown in FIG. 1A, 1B and Table 2 indicate that the binding EC50 values of the affinity matured single-point mutated antibodies against CD24 (27-56aa)-GST (NEOX-4) were lower than the EC50 value of the corresponding humanized antibody huG14-1, indicating that the experimental single-point mutation affinity matured CD24 antibodies have strong affinities against CD24 (27-56aa)-GST (NEOX-4).

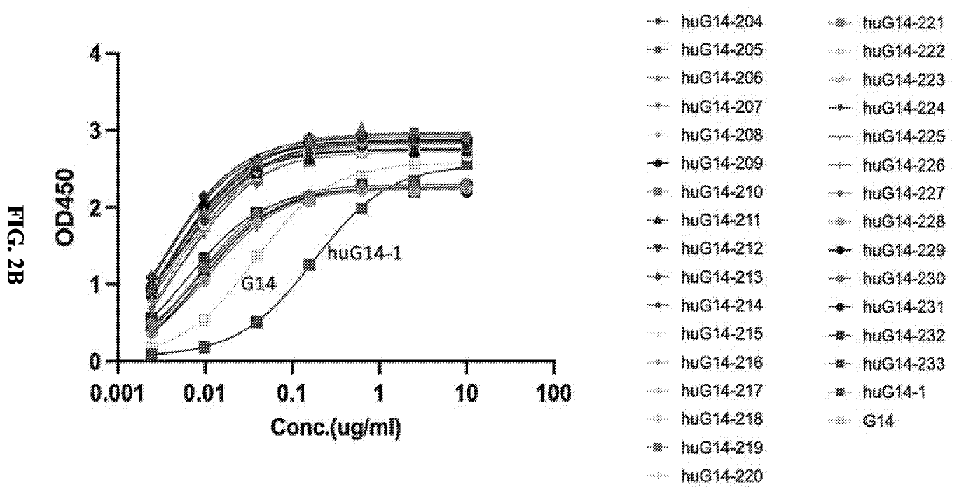

The results as shown in FIG. 2A, 2B and Tables 3-1 and 3-2 indicate that the binding EC50 values of the affinity matured single-point mutant antibodies against CD24 (27-56aa)-GST (NEOX-4) were lower than the EC50 value of humanized antibody huG14-1 and chimeric antibody G14, indicating that the combined mutation affinity matured CD24 antibodies have strong affinities against CD24 (27-56aa)-GST (NEOX-4).

TABLE 2

| | EC50 (μg/ml) |
|---|---|
| huG14-101 | 0.03246 |
| huG14-102 | 0.00961 |
| huG14-103 | 0.008126 |
| huG14-104 | 0.01826 |
| huG14-105 | 0.02057 |
| huG14-106 | 0.01574 |
| huG14-107 | 0.04943 |
| huG14-108 | 0.07843 |
| huG14-1 | 0.128 |
| G14 | 0.02482 |
| huG14-109 | 0.01092 |
| huG14-110 | 0.01534 |
| huG14-111 | 0.02928 |
| huG14-1 | 0.136 |
| G14 | 0.009884 |

TABLE 3-1
Binding EC50 Values of CD24 antibodies (combined mutations) for CD24 by ELISA

| | huG14-112 | huG14-113 | huG14-114 | huG14-115 | huG14-116 | huG14-117 | huG14-118 |
|---|---|---|---|---|---|---|---|
| EC50(μg/ml) | 0.01208 | 0.01186 | 0.01041 | 0.01288 | 0.01125 | 0.00727 | 0.004831 |
| | huG14-119 | huG14-120 | huG14-121 | huG14-121 | huG14-122 | huG14-123 | huG14-124 |
| EC50(μg/ml) | 0.008934 | 0.006228 | 0.007042 | 0.007368 | 0.00712 | 0.00386 | 0.004061 |
| | huG14-125 | huG14-126 | huG14-127 | huG14-128 | huG14-129 | huG14-130 | huG14-132 |
| EC50(μg/ml) | 0.006186 | 0.003933 | 0.002971 | 0.004123 | 0.004087 | 0.003889 | 0.004718 |
| | huG14-133 | huG14-134 | huG14-135 | huG14-136 | huG14-137 | huG14-138 | huG14-139 |
| EC50(μg/ml) | 0.005655 | 0.003832 | 0.003866 | 0.004053 | 0.003866 | 0.003279 | 0.003166 |

| | huG14-1 | G14 |
|---|---|---|
| EC50(μg/ml) | 0.1543 | 0.03097 |

TABLE 3-2
Binding EC50 Values for of CD24 antibodies (combined mutations) for CD24 by ELISA

| | huG14-204 | huG14-205 | huG14-206 | huG14-207 | huG14-208 | huG14-209 | huG14-210 |
|---|---|---|---|---|---|---|---|
| EC50(μg/ml) | 0.00667 | 0.006276 | 0.004925 | 0.005712 | 0.004015 | 0.004126 | 0.004261 |
| | huG14-211 | huG14-212 | huG14-213 | huG14-214 | huG14-215 | huG14-216 | huG14-217 |
| EC50(μg/ml) | 0.003341 | 0.003185 | 0.00329 | 0.003061 | 0.003412 | 0.004397 | 0.004432 |
| | huG14-218 | huG14-219 | huG14-220 | huG14-221 | huG14-222 | huG14-223 | huG14-224 |
| EC50(μg/ml) | 0.004713 | 0.005688 | 0.00517 | 0.005933 | 0.004907 | 0.004821 | 0.006178 |
| | huG14-225 | huG14-226 | huG14-227 | huG14-228 | huG14-229 | huG14-230 | huG14-231 |
| EC50(μg/ml) | 0.006276 | 0.01071 | 0.007626 | 0.009882 | 0.0091 | 0.008892 | 0.008249 |

| | huG14-232 | huG14-233 | huG14-1 | G14 |
|---|---|---|---|---|
| EC50(μg/ml) | 0.006208 | 0.008407 | 0.1774 | 0.0366 |

### Example 8-Determination of the Binding of Chimeric Antibody G14 and Affinity Matured CD24 Antibody to Tumor Cells Highly Expressing CD24

NALM6 and MDA-MB-486 are human tumor cell lines that express high levels of CD24. The CD24 antibodies were co-incubated with NALM6 and MDA-MB-486 cells to determine whether the CD24 antibodies can specifically bind to NALM6 and MDA-MB-486 cells. The control positive antibody was P6373, the sequence of which is shown in SEQ ID NO 603 and 604 (see WO 2019/222082 A1). The process for cell binding experiment was as follows: adding 5×10E4 cells to each well of a 96-well plate, and removing the supernatant after centrifugation; diluting the CD24 antibody with PBS: starting from the initial concentration of 30 μg/mL, with a 4-fold gradient dilution, with a total of 7 dilution gradients; adding 100 μl of the diluted CD24 antibodies to each well of the 96-well plate, resuspending the cells, and then incubating at 4 °C for 1 hour; removing the supernatant by centrifugation, washing the cells with 200 μl staining buffer (1×PBS+1% BSA) for 3 times, and then adding 50 μl of a 1:200 diluted fluorescent secondary antibody goat-anti-human-IgG (Fc)-PE (Jackson, Cat No. 109-116-098) to each well; after resuspending, incubating in the dark at 4 °C for 30 min; washing the cells with 200 μl staining buffer (1×PBS+1% BSA) for 3 times, resuspending the cells with 50 μl staining buffer (1×PBS+1% BSA), and placing the cells onto a Beckman cyto FLFX for detection and data analysis.

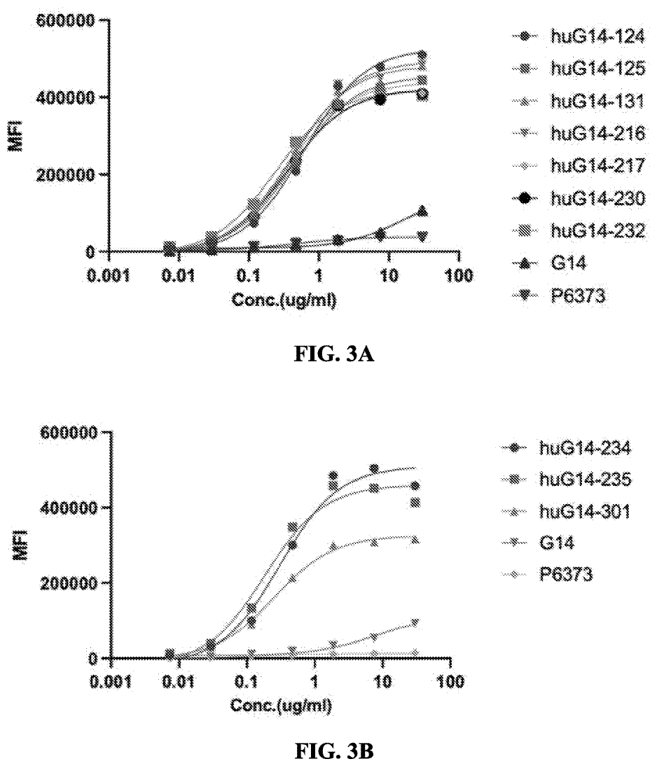

As shown in FIG. 3A, 3B and Table 4 both the chimeric antibody G14 and the affinity matured CD24 antibodies could specifically bind to the CD24 protein on the surface of NALM6 cells, and the binding activities of the affinity matured CD24 antibodies were further significantly higher than that of the chimeric antibody G14.

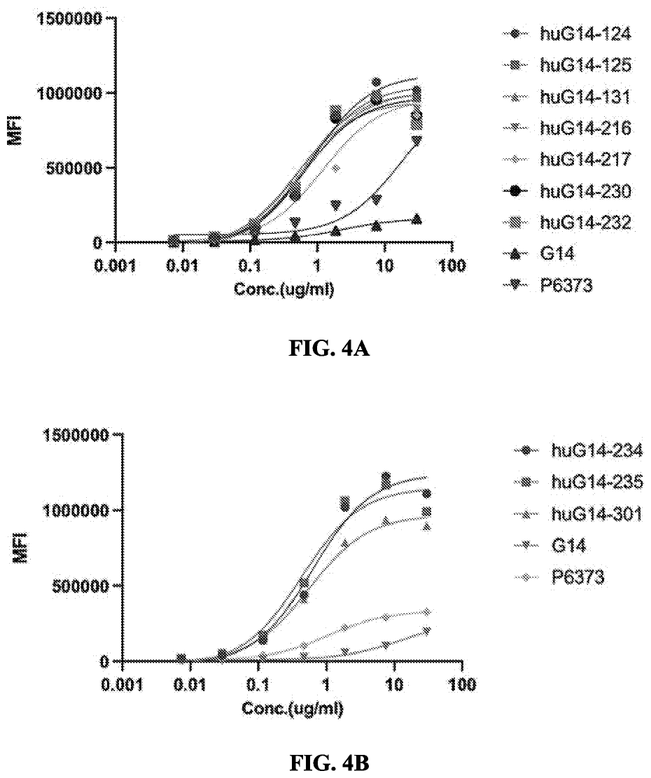

As shown in FIG. 4A, 4B and Table 5 both the chimeric antibody G14 and the affinity matured CD24 antibodies could specifically bind to the CD24 protein on the surface of MDA-MB-468 cells, and the binding activities of the affinity matured CD24 antibodies were further significantly higher than that of the chimeric antibody G14. The above results show that the antibodies of the present invention can effectively bind to CD24 on the surface of tumor cells.

TABLE 4

| | EC50 (μg/ml) |
|---|---|
| huG14-124 | 0.6089 |
| huG14-125 | 0.4426 |
| huG14-131 | 0.3913 |
| huG14-216 | 0.4315 |
| huG14-217 | 0.3484 |
| huG14-230 | 0.35 |
| huG14-232 | 0.2355 |
| G14 | 18.46 |
| P6373 | 0.4688 |
| huG14-234 | 0.3052 |
| huG14-235 | 0.184 |
| huG14-301 | 0.2555 |
| G14 | 7.296 |
| P6373 | 0.2175 |

TABLE 5

| | EC50 (μg/ml) |
|---|---|
| huG14-234 | 0.3052 |
| huG14-235 | 0.184 |
| huG14-301 | 0.2555 |
| G14 | 7.296 |
| P6373 | 0.2175 |
| huG14-124 | 0.8269 |
| huG14-125 | 0.6836 |
| huG14-131 | 0.5697 |
| huG14-216 | 0.6758 |
| huG14-217 | 1.211 |
| huG14-230 | 0.6304 |
| huG14-232 | 0.4843 |
| G14 | 2.241 |
| P6373 | 17.95 |

### Example 9—Determination of Biacore Affinities of Chimeric Antibody and Affinity Matured Antibodies Against CD24 Protein

A biotinylated CD24 protein (ACRO, Cat No. CD4-H82E9) was coupled to a CM5 biosensor chip (Cytiva, BR100530), and then 2-fold dilutions of CD24 antibodies at different concentrations were passed on the surface of the chip. The reaction signals were detected in real time using Biacore (Cytiva, Biacore 8K) to obtain the association (ka) and dissociation (kd) curves, and then the affinity (K_{D}) values were obtained by fitting, as shown in the table X below. The results show that the binding affinities of affinity-matured antibodies huG14-234, huG14-235 and huG14-301 against CD24 protein (ACRO, Cat No. CD4-H82E9) were significantly higher than that of the chimeric antibody G14.

| | Kₐ (1/Ms) | K_{d} (1/s) | K_{D} (M) |
|---|---|---|---|
| G14 | 1.08E+06 | 1.31E−02 | 1.21E−08 |
| huG14-234 | 1.21E+06 | 4.38E−03 | 3.62E−09 |
| huG14-235 | 1.57E+06 | 4.21E−03 | 2.69E−09 |
| huG14-301 | 6.05E+05 | 4.58E−03 | 7.58E−09 |

### Example 10—Evaluation of ADCC Activity

Examples 10-17 of the present invention involve three CD24 antibodies for comparison, and their full-length sequences are respectively described as follows: SWA11 (light chain: SEQ ID NO: 601, heavy chain: SEQ ID NO: 602), P6373 (light chain: SEQ ID NO: 603, heavy chain: SEQ ID NO: 604) and IMM47H (light chain: SEQ ID NO: 605, heavy chain: SEQ ID NO: 606).

The Jurkat cell line containing the FcγRIIIA luciferase reporter gene and the target cells NALM6 or MDA-MB-468 were inoculated into 96-well plates at an effect-target ratio of 6:1, and then 3-fold gradient dilutions of antibodies to be tested at a starting concentration of 20 μg/ml and Isotype (anti-HEL IgG1 isotype control antibody) were added. After incubation at 37 °C for 5 h, luciferase substrate was added and the reaction was allowed to proceed for 5 min, and the fluorescence intensity was detected by a microplate reader.

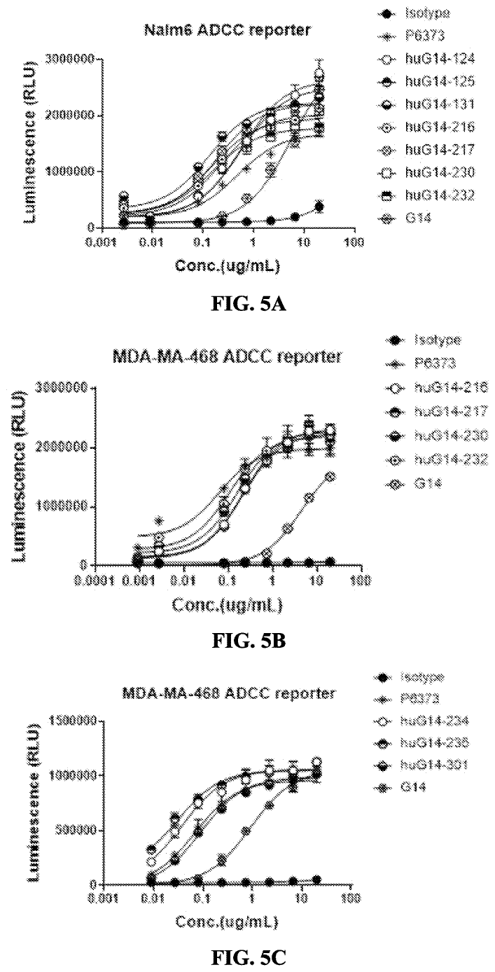

FIG. 5A, 5B, 5C, and Table 6 show that the antibodies huG14-124, huG14-125, huG14-131, huG14-216, huG14-217, huG14-230, huG14-232, huG14-234, huG14-235, huG14-301 all possess ADCC activity against NALM6 and MDA-MB-468.

TABLE 6

| | EC50 (ug/ml) |
|---|---|
| huG14-124 | 0.57 |
| huG14-125 | 0.53 |
| huG14-131 | 0.13 |
| huG14-216 | 0.21 |
| huG14-217 | 0.18 |
| huG14-230 | 0.15 |
| huG14-232 | 0.15 |
| G14 | 5.52 |
| P6373 | 0.48 |
| huG14-216 | 0.21 |
| huG14-217 | 0.20 |
| huG14-230 | 0.16 |
| huG14-232 | 0.10 |
| G14 | 4.55 |
| P6373 | 0.08 |
| huG14-234 | 0.03 |
| huG14-235 | 0.03 |
| huG14-301 | 0.08 |
| G14 | 0.9 |
| P6373 | 0.07 |

### Example 11—Evaluation of ADCP Activity

Monocytes were isolated from PBMCs and were induced to differentiate into M2 macrophages with M-CSF, TGFβ, and IL10 for later use. The tumor cells NALM6 and MDA-MB-468 were labeled with CFSE and were mixed with M2 macrophages at a ratio of 1:1. Then 5-fold gradient dilutions of the anti-CD24 antibody G14 and its affinity matured sequences at a starting concentration of 5 μg/ml were added. After incubating for 4 h at room temperature, cells were washed and CD11b flow staining was performed. The ratios of respective cell subpopulations were detected by flow cytometer. Phagocytosis efficiency=CFSE+CD11b+/CD11b+.

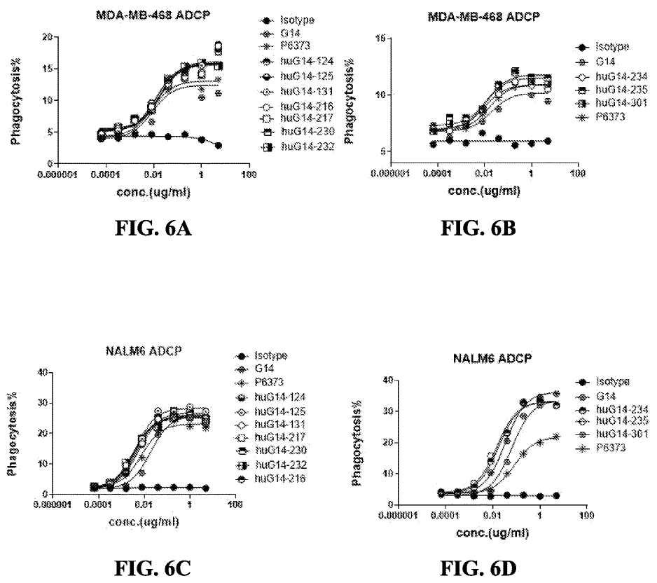

FIG. 6A, 6B, 6C, 6D and Table 7 show that all the tested antibodies showed tumor phagocytic activity against NALM6 and MDA-MB-468.

TABLE 7

| | EC50 (ug/ml) |
|---|---|
| huG14-234 | 0.009 |
| huG14-235 | 0.012 |
| huG14-301 | 0.01 |
| G14 | 0.019 |
| P6373 | 0.015 |
| huG14-124 | 0.005 |
| huG14-125 | 0.004 |
| huG14-131 | 0.003 |
| huG14-216 | 0.003 |
| huG14-217 | 0.003 |
| huG14-230 | 0.004 |
| huG14-232 | 0.003 |
| G14 | 0.018 |
| P6373 | 0.007 |
| huG14-234 | 0.017 |
| huG14-235 | 0.014 |
| huG14-301 | 0.03 |
| G14 | 0.06 |
| P6373 | 0.08 |

### Example 12—CD24 Antibodies of the Description Show Reduced Binding to B Cells and Activated T Cells

CD24 protein is expressed in both normal B cells and activated T cells. Flow cytometry was used to study the binding profile of CD24 antibodies to B cells and activated T cells. Serial dilutions of CD24 antibodies were incubated with normal primary B cells or with T cells that were activated with CD3, CD28 for three days at 4 °C for 1 h, respectively, and then washed with PBS for three times. Then, a 1:200 diluted fluorescent secondary antibody Goat-anti-human-IgG (Fc)-PE was added to each well, and incubated at 4 °C for 30 min. After washing with PBS for three times, the cells were resuspended and subjected to flow cytometer detection by CytoFLEX.

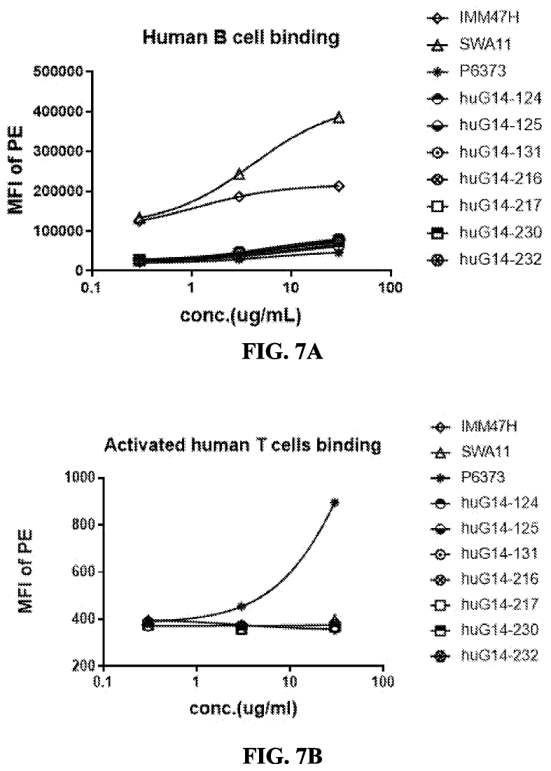

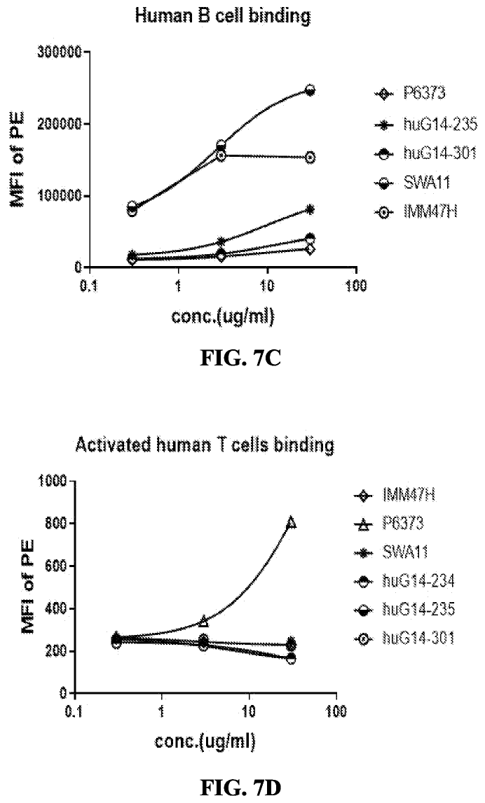

FIG. 7A, 7B, 7C, 7D and Table 8 show that huG14-124, huG14-125, huG14-131, huG14-216, huG14-217, huG14-230, huG14-232, huG14-234, huG14-235, and huG14-301 did not bind to primary B cells and activated T cells. However, IMM47H and SWA11 showed significant binding to B cells and P6373 showed significant binding to activated T cells. This is potentially important as the antibodies of the present invention may possess significant safety advantages to previously described anti-CD34 antibodies.

TABLE 8

| | B cell | Activated T cell |
|---|---|---|
| | binding | binding |
| huG14-124 | − | − |
| huG14-125 | − | − |
| huG14-131 | − | − |
| huG14-216 | − | − |
| huG14-217 | − | − |
| huG14-230 | − | − |
| huG14-232 | − | − |
| huG14-234 | − | − |
| huG14-235 | − | − |
| huG14-301 | − | − |
| IMM47H | + | − |
| SWA11 | + | − |
| P6373 | − | + |

### Example 13—Tumor Cell Specific Elimination

To evaluate the tumor specific elimination activity of anti-CD24 antibodies, 1×10⁵ CFSE labeled NALM6 cells were mixed with human PBMCs at a ratio of 1:7. Then anti-CD24 antibody G14, SWA11, IMM47H, P6373 were added into the NALM6/PMBCs mixture at a final concentration of 10 μg/ml. After 4 h incubation at 37 °C, 5% CO₂, cell mixtures were stained with PE labeled anti-human CD19 antibody at 4 °C in dark for 30 min. washing the cells with 200 μl staining buffer (1×PBS+1% BSA) for 3 times, resuspending the cells with 50 μl staining buffer (1×PBS+1% BSA), and placing the cells onto a Beckman cyto FLFX for detection and data analysis. NALM6 elimination rate (%)=1−(CFSE+CD19+ cells in anti-CD24 treated group/CFSE+CD19+ cell in isotype control group)×100%, B cells elimination rate (%)=1-(anti-CD24 treatment group CD19+CFSE-B cell percentage in PBMCs/isotype control group CD19+CFSE-B cell percentage in PBMCs)×100%.

As shown in Table 9, huG14-301 and P6373 can selectively eliminate tumor cells (NALM6) and spare the normal B cells in PBMCs. However, the reference antibodies SWA11, IMM47H kill both NALM6 and normal B cells. Those results indicate that G14 may have better safety profile over previously described anti-CD24 antibodies.

TABLE 9

| | NALM6 | B cells elimination |
|---|---|---|
| | elimination rate (%) | rate (%) |
| huG14-301 | 91.1 | 0.1 |
| P6373 | 67.8 | 3.5 |
| IMM47H | 93.3 | 28.4 |
| SWA11 | 87.5 | 72.1 |

### Example 14—Glycosyl Selectivity

CD24 is a highly glycosylated protein, and the affinity of antibodies against CD24 proteins with different degrees of glycosylation will vary. Studies and literatures have shown that the glycosylation of CD24 in tumor cells is different from that in normal cells. Usually, the glycosylation of CD24 in tumor cells is reduced to a certain extent, thereby exposing the amino acid residues of its protein (for such literature, see, for example, WO2019222082A1, etc.).

The binding abilities of antibodies to CD24 with different glycosylation levels were determined by ELISA. The method was as follows: 1) digesting CD24 (27-56aa)-GST (NEOX-4) with N-Glycanase, Neuraminidase or N-Glycanase+Neuraminidase, respectively; 2) diluting the CD24 before and after digestion with PBS to 1 μg/ml and then coating the 96-well plate with the CD24 at 4 °C overnight; 3) after blocking with BSA, adding 0.78, 0.19, 0.05 μg/ml of G14 antibodies and incubating at 37 °C for 1 h; 4) adding secondary antibody: washing the plate with PBS for 3 times, diluting the HRP-labeled anti-human IgG Fc antibody (Jackson, Cat No. 109-035-098) for 5000 times, adding 100 μl of which to each well, and incubating at 37 °C for 1 h; 5) color development: washing the plate with PBS for 6 times, adding 50 μl TMB (Beijing Zhizhou Biotechnology Co., Ltd., Cat No. ZZ180001) to each well, and placing the plate in the dark at room temperature for 10 minutes, then adding 50 μl of 2M H2SO4 to terminate the color development reaction.

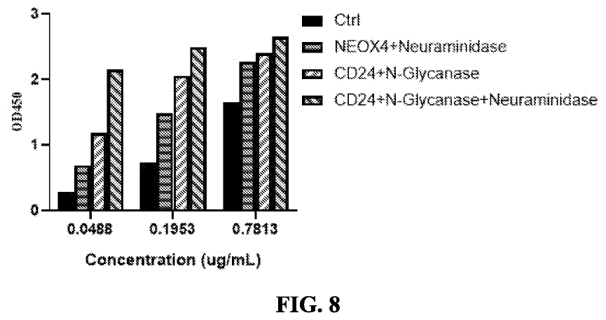

FIG. 8 shows that the affinity of G14 against NEOX-4 protein digested by N-Glycanase, Neuraminidase or N-Glycanase+Neuraminidase was higher than that before digestion.

Cell preparation: the cells (NALM6, MDA-MB-468, primary B cells) were counted (cell viability 98%), centrifuged and resuspended in buffer, and aliquoted into a 96-well conical bottom plate at 5×10⁵, 200 μl per well. After centrifugation, the supernatant was removed and the cells were resuspended in blank medium. N-Glycanase, Neuraminidase, N-Glycanase+Neuraminidase were added, respectively, and then placed into an incubator for 3 h at 37 °C. The cells were washed with PBS for three times and then resuspended with PBS containing 2 ug/ml of huG14-235 or huG14-301, and then incubated at room temperature for 1 h. After washing 3 times with PBS, fluorescent secondary antibody staining was performed and fluorescence intensities were detected by CytoFLEX.

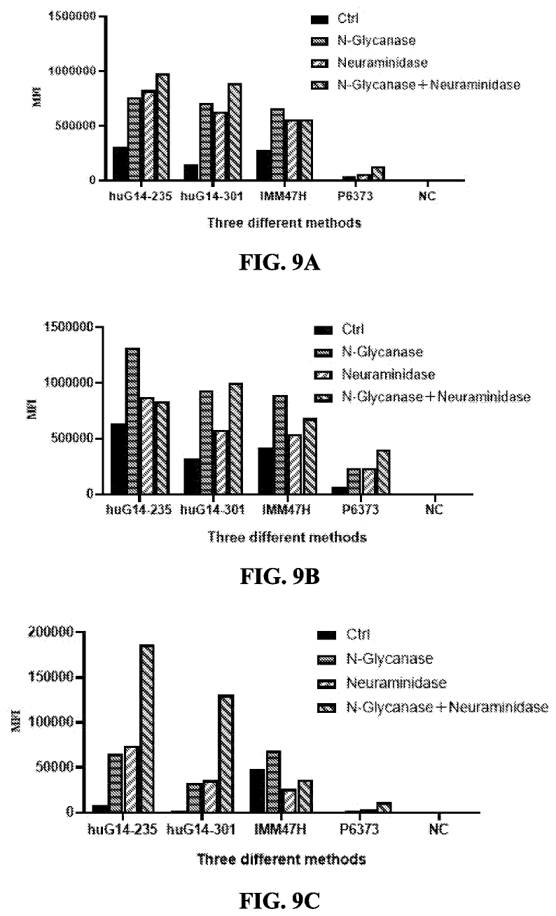

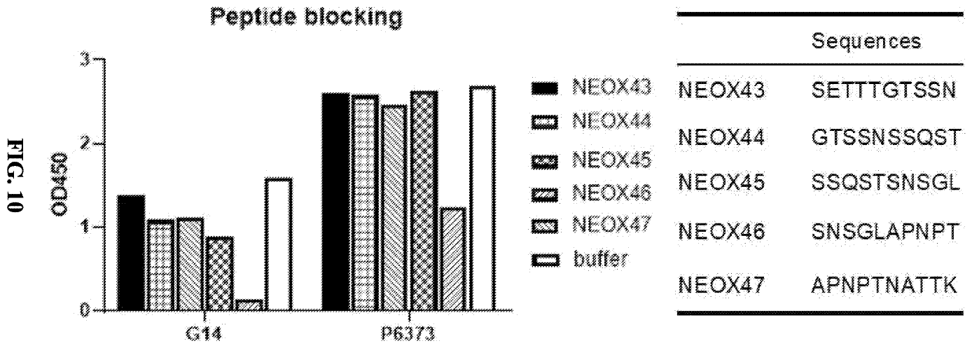

FIGS. 9A, 9B, and 9C show that huG14-235 or huG14-301 had strong binding to NALM6 and MDA-MB-468, and when tumor cells were treated with N-Glycanase, Neuraminidase or N-Glycanase+Neuraminidase, the binding of the antibodies to NALM6, MDA-MB-468 was significantly enhanced. The huG14-235 or huG14-301 did not bind to primary B cells, but could bind to primary B cells after de-glycosylation by enzymatic digestion.

### Example 15—Analysis of CD24 Antibody Binding Epitope

Different partially overlapping truncations of CD24 extracellular segments (NEOX43-NEOX47) were synthesized to study the precise epitope of CD24 antibody which binds to the antigen. First, a 96-well plate was coated with 1 ug/mg of CD24 antigen at 4 °C overnight, and blocked by BSA at room temperature for 1 h, then the CD24 antibodies were mixed with 10 ug/ml of different truncations (NEOX43-NEOX47) respectively and then added to the 96-well plate for incubation at room temperature for 1 h. After washing 3 times with PBS, anti-hFc HRP secondary antibody was added for incubation, and the reaction substrate was added for detection. Results shown in FIG. 10 show that both G14 and P6373 bound to the NEOX46: SNSGLAPNPT peptide segment.

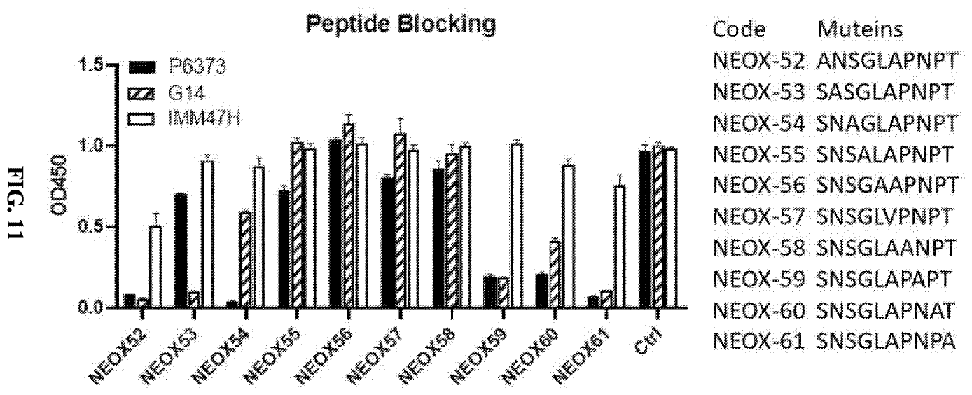

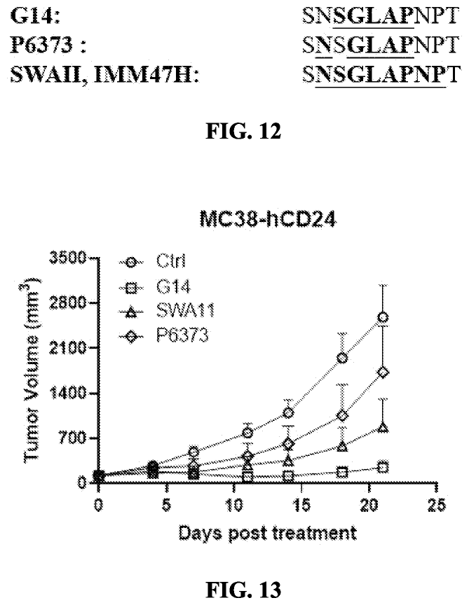

In order to further study the specific amino acid sites bound by CD24 antibodies, a series of single point mutations (NEOX52-NEOX61) were designed for NEOX46: SNSGLAPNPT, and then the same method was used to study the binding of the antibodies to different peptide segments. Results shown in FIG. 11 show that antibodies such as G14, P6373 differed in the specific amino acid binding sites of the epitope peptide segment SNSGLAPNPT. G14 binded to CD24 at 44S, 45G, 46L, 47A, 48P, while P6373 binded at 43N, 45G, 46L, 47A, 48P (the numbering of residues is based on the full-length CD24 sequence with the signal peptide, as shown in SEQ ID NO: 502). For further details, see FIG. 12 (Binding Epitope Comparison).

### Example 16—Pharmacodynamic Study of CD24 Antibodies in Colon Cancer Mouse Model

The hSiglec10 transgenic mice were subcutaneously inoculated with 1×10⁶ MC38-hCD24 tumor cells, and when the tumor volumes reached to 150 mm³, they were administered with CD24 antibodies intraperitoneally for treatment. The dose was 20 mg/kg twice a week for two consecutive weeks. The tumor volumes were measured twice a week and the tumor growth curve was plotted. The results in FIG. 13 show that the G14 antibody showed significant tumor-suppressive activity with a tumor suppression rate of 90%.

### Example 17—Pharmacodynamic Study of CD24 Antibodies in Hematological Tumor Model

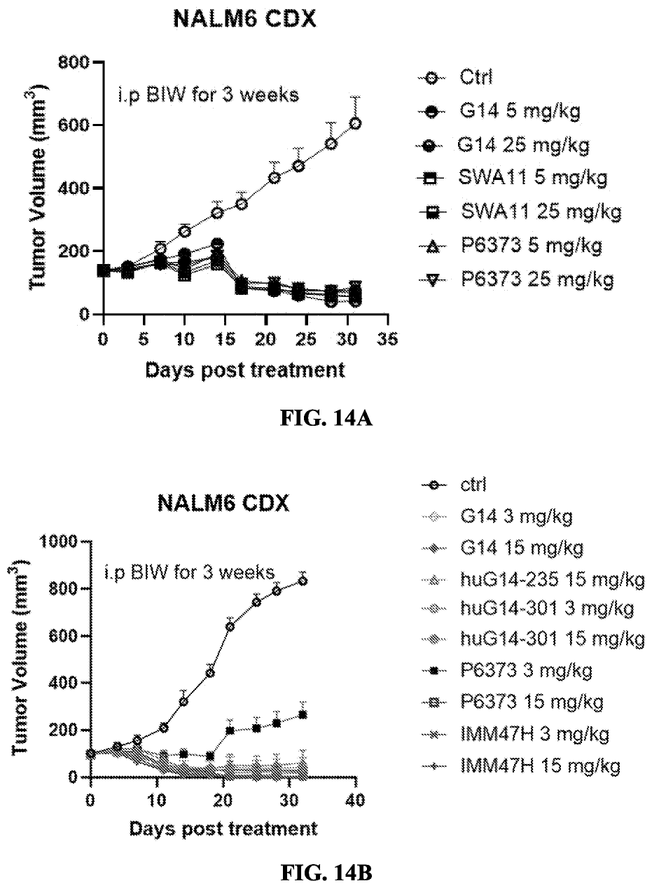

Nude mice aged 6-8 weeks were subcutaneously inoculated with 5×10⁶ NALM6 tumor cells, and when the tumor volumes reached to 150 mm³, the mice were administered with different doses of CD24 antibodies intraperitoneally for treatment. The mice were administered twice a week for three consecutive weeks. The tumor volumes were measured twice a week and the tumor growth curve was plotted. The results in FIGS. 14A and 14B show that both low-dose and high-dose of G14, huG14-235 and huG14-301 antibodies showed significant tumor-suppressive activity with both tumor inhibition rates greater than 90%.

### Example 18—Pharmacodynamic Study of CD24 Antibodies in Breast Cancer

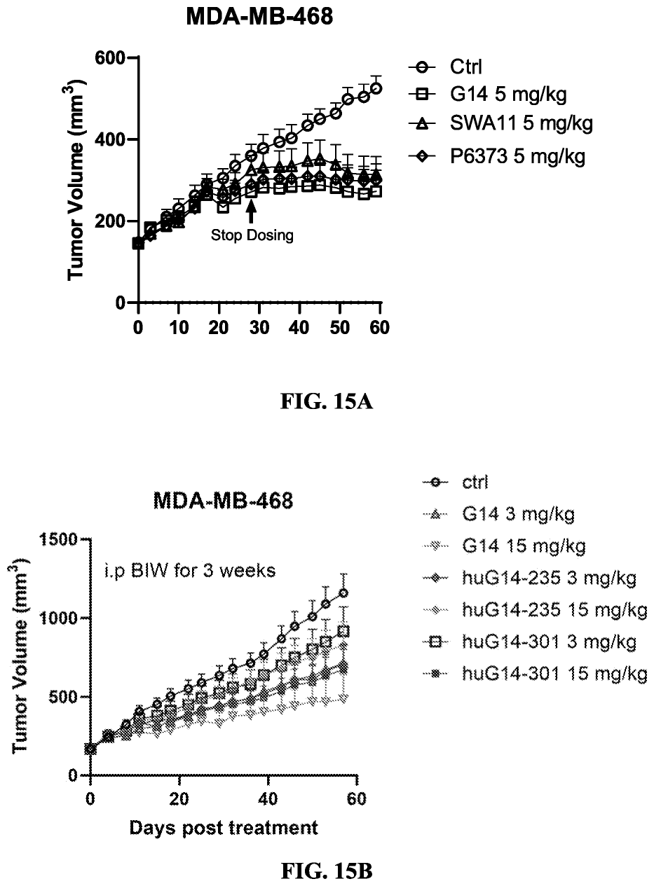

Nude mice aged 6-8 weeks were subcutaneously inoculated with 1×10⁷ triple-negative breast cancer MDA-MB-468 cells, and when the tumor volume reached to 150 mm³, the mice were administered with CD24 antibodies intraperitoneally for treatment. The mice were administered twice a week for four consecutive weeks (FIG. 15A) or three consecutive weeks (FIG. 15B). The tumor volumes were measured twice a week and the tumor growth curve was plotted. The results in FIGS. 15A and 15B show that the G14, huG14-235 and huG14-301 antibodies antibody showed significant tumor-suppressive activity (G14 with a tumor suppression rate of 66%).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. An antibody, or antigen binding fragment thereof, that binds to CD24, wherein the antibody does not bind to T lymphocytes.

2. The antibody of claim 1, wherein the antibody does not bind to activated T lymphocytes.

3. The antibody of claim 1 or 2, wherein the antibody does not bind to B lymphocytes.

4. The antibody of any one of claims 1 to 3, wherein the antibody binds to CD24 with a K_{D} of less than 1×10⁻⁸.

5. The antibody of any one of claims 1 to 3, wherein the antibody binds to CD24 with a K_{D} of less than 1×10⁻⁹.

6. The antibody of any one of claims 1 to 5, wherein the antibody is a humanized or chimeric antibody.

7. The antibody of any one of claims 1 to 6, wherein the antibody is a Fab, F (ab) 2, or a single chain variable fragment (scFv).

8. The antibody of any one of claims 1 to 6, wherein the antibody is a heavy chain antibody or antigen binding fragment thereof.

9. The antibody of any one of claims 1 to 6, wherein the antibody is an IgG antibody.

10. The antibody of claim 9, wherein the antibody is an IgG₁ isotype.

11. The antibody of claim 9, wherein the antibody is an IgG₄ isotype.

12. The antibody of any one of claims 1 to 11, wherein the antibody binds to the serine at residue 18 of SEQ ID NO: 500.

13. The antibody of any one of claims 1 to 11, wherein the antibody requires the presence of a serine at residue 18 of SEQ ID NO: 500 for binding.

14. The antibody of any one of claims 1 to 13, wherein the antibody binds to an epitope consisting of the amino acid sequence of SEQ ID NO 501.

15. An antibody, or antigen binding fragment thereof, that binds to CD24, wherein the antibody or antigen binding fragment thereof comprises:
a. a light chain complementarity determining region 1 (LCDR1) comprising the amino acid sequence set forth in SEQ ID NO: 401;
b. a light chain complementarity determining region 2 (LCDR2) comprising the amino acid sequence set forth in SEQ ID NO: 403;
c. a light chain complementarity determining region 3 (LCDR3) comprising the amino acid sequence set forth in SEQ ID NO: 405;
d. a heavy chain complementarity determining region 1 (HCDR1) comprising the amino acid sequence set forth in SEQ ID NO: 407;
e. a heavy chain complementarity determining region 2 (HCDR2) comprising the amino acid sequence set forth in SEQ ID NO: 409; and/or
f. a heavy chain complementarity determining region 3 (HCDR3) comprising the amino acid sequence set forth in SEQ ID NO: 411.

16. The antibody of any one of claims 1 to 15, wherein the antibody comprises:
a. a light chain complementarity determining region 1 (LCDR1) comprising the amino acid sequence set forth in SEQ ID NO: 401;
b. a light chain complementarity determining region 2 (LCDR2) comprising the amino acid sequence set forth in SEQ ID NO: 403;
c. a light chain complementarity determining region 3 (LCDR3) comprising the amino acid sequence set forth in SEQ ID NO: 405;
d. a heavy chain complementarity determining region 1 (HCDR1) comprising the amino acid sequence set forth in SEQ ID NO: 407;
e. a heavy chain complementarity determining region 2 (HCDR2) comprising the amino acid sequence set forth in SEQ ID NO: 409; and/or
f. a heavy chain complementarity determining region 3 (HCDR3) comprising the amino acid sequence set forth in SEQ ID NO: 411.

17. The antibody of any one of claims 1 to 15, wherein the antibody comprises:
a. a light chain complementarity determining region 1 (LCDR1) comprising the amino acid sequence set forth in SEQ ID NO: 402;
b. a light chain complementarity determining region 2 (LCDR2) comprising the amino acid sequence set forth in SEQ ID NO: 403;
c. a light chain complementarity determining region 3 (LCDR3) comprising the amino acid sequence set forth in SEQ ID NO: 406;
d. a heavy chain complementarity determining region 1 (HCDR1) comprising the amino acid sequence set forth in SEQ ID NO: 408;
e. a heavy chain complementarity determining region 2 (HCDR2) comprising the amino acid sequence set forth in SEQ ID NO: 409; and/or
f. a heavy chain complementarity determining region 3 (HCDR3) comprising the amino acid sequence set forth in SEQ ID NO: 412.

18. The antibody of any one of claims 1 to 15, wherein the antibody comprises:
a. a light chain complementarity determining region 1 (LCDR1) as set forth in SEQ ID NO: 402;
b. a light chain complementarity determining region 2 (LCDR2) comprising the amino acid sequence set forth in SEQ ID NO: 403;
c. a light chain complementarity determining region 3 (LCDR3) comprising the amino acid sequence set forth in SEQ ID NO: 406;
d. a heavy chain complementarity determining region 1 (HCDR1) comprising the amino acid sequence set forth in SEQ ID NO: 408;
e. a heavy chain complementarity determining region 2 (HCDR2) comprising the amino acid sequence set forth in SEQ ID NO: 409; and/or
f. a heavy chain complementarity determining region 3 (HCDR3) comprising the amino acid sequence set forth in SEQ ID NO: 412.

19. The antibody of any one of claims 1 to 15, wherein the antibody comprises:
a. a light chain complementarity determining region 1 (LCDR1) comprising an amino acid sequence selected from the group consisting of SEQ ID NO: 13, 23, 33, 43, 53, 63, 73, 83, 703, 713, 723, 733, and 743;
b. a light chain complementarity determining region 2 (LCDR2) comprising an amino acid sequence selected from the group consisting of SEQ ID NO: 14, 24, 34, 44, 54, 64, 74, 84, 704, 714, 724, 734, and 744;
c. a light chain complementarity determining region 3 (LCDR3) comprising an amino acid sequence selected from the group consisting of SEQ ID NO: 15, 25, 35, 45, 55, 65, 75, 85, 705, 715, 725, 735, and 745;
d. a heavy chain complementarity determining region 1 (HCDR1) comprising an amino acid sequence selected from the group consisting of SEQ ID NO: 16, 26, 36, 46, 56, 66, 76, 86, 706, 716, 726, 736, and 746;
e. a heavy chain complementarity determining region 2 (HCDR2) comprising an amino acid sequence selected from the group consisting of SEQ ID NO: 17, 27, 37, 47, 57, 67, 77, 87, 707, 717, 727, 737, and 747; and/or
f. a heavy chain complementarity determining region 3 (HCDR3) comprising an amino acid sequence selected from the group consisting of SEQ ID NO: 18, 28, 38, 48, 58, 68, 78, 88, 708, 718, 728, 738, and 748.

20. The antibody of any one of claims 1 to 15, wherein the antibody comprises a combination of LCDR1, LCDR2, and LCDR3 and HCDR1, HCDR2, and HCDR3, wherein the combination is selected from the group consisting of:
(i) LCDR1 comprising SEQ ID NO: 13, LCDR2 comprising SEQ ID NO: 14, LCDR3 comprising SEQ ID NO: 15, HCDR1 comprising SEQ ID NO: 16, HCDR2 comprising SEQ ID NO: 17, HCDR3 comprising SEQ ID NO: 18;
(ii) LCDR1 comprising SEQ ID NO: 23, LCDR2 comprising SEQ ID NO: 24, LCDR3 comprising SEQ ID NO: 25, HCDR1 comprising SEQ ID NO: 26, HCDR2 comprising SEQ ID NO: 27, HCDR3 comprising SEQ ID NO: 28;
(iii) LCDR1 comprising SEQ ID NO: 33, LCDR2 comprising SEQ ID NO: 34, LCDR3 comprising SEQ ID NO: 35, HCDR1 comprising SEQ ID NO: 36, HCDR2 comprising SEQ ID NO: 37, HCDR3 comprising SEQ ID NO: 38;
(iv) LCDR1 comprising SEQ ID NO: 43, LCDR2 comprising SEQ ID NO: 44, LCDR3 comprising SEQ ID NO: 45, HCDR1 comprising SEQ ID NO: 46, HCDR2 comprising SEQ ID NO: 47, HCDR3 comprising SEQ ID NO: 48;
(v) LCDR1 comprising SEQ ID NO: 53, LCDR2 comprising SEQ ID NO: 54, LCDR3 comprising SEQ ID NO: 55, HCDR1 comprising SEQ ID NO: 56, HCDR2 comprising SEQ ID NO: 57, HCDR3 comprising SEQ ID NO: 58;
(vi) LCDR1 comprising SEQ ID NO: 63, LCDR2 comprising SEQ ID NO: 64, LCDR3 comprising SEQ ID NO: 65, HCDR1 comprising SEQ ID NO: 66, HCDR2 comprising SEQ ID NO: 67, HCDR3 comprising SEQ ID NO: 68;
(vii) LCDR1 comprising SEQ ID NO: 73, LCDR2 comprising SEQ ID NO: 74, LCDR3 comprising SEQ ID NO: 75, HCDR1 comprising SEQ ID NO: 76, HCDR2 comprising SEQ ID NO: 77, HCDR3 comprising SEQ ID NO: 78;
(viii) LCDR1 comprising SEQ ID NO: 83, LCDR2 comprising SEQ ID NO: 84, LCDR3 comprising SEQ ID NO: 85, HCDR1 comprising SEQ ID NO: 86, HCDR2 comprising SEQ ID NO: 87, HCDR3 comprising SEQ ID NO: 88;
(ix) LCDR1 comprising SEQ ID NO: 703, LCDR2 comprising SEQ ID NO: 704, LCDR3 comprising SEQ ID NO: 705, HCDR1 comprising SEQ ID NO: 706, HCDR2 comprising SEQ ID NO: 707, HCDR3 comprising SEQ ID NO: 708;
(x) LCDR1 comprising SEQ ID NO: 713, LCDR2 comprising SEQ ID NO: 714, LCDR3 comprising SEQ ID NO: 715, HCDR1 comprising SEQ ID NO: 716, HCDR2 comprising SEQ ID NO: 717, HCDR3 comprising SEQ ID NO: 718;
(xi) LCDR1 comprising SEQ ID NO: 723, LCDR2 comprising SEQ ID NO: 724, LCDR3 comprising SEQ ID NO: 725, HCDR1 comprising SEQ ID NO: 726, HCDR2 comprising SEQ ID NO: 727, HCDR3 comprising SEQ ID NO: 728;
(xii) LCDR1 comprising SEQ ID NO: 733, LCDR2 comprising SEQ ID NO: 734, LCDR3 comprising SEQ ID NO: 735, HCDR1 comprising SEQ ID NO: 736, HCDR2 comprising SEQ ID NO: 737, HCDR3 comprising SEQ ID NO: 738; and
(xiii) LCDR1 comprising SEQ ID NO: 743, LCDR2 comprising SEQ ID NO: 744, LCDR3 comprising SEQ ID NO: 745, HCDR1 comprising SEQ ID NO: 746, HCDR2 comprising SEQ ID NO: 747, HCDR3 comprising SEQ ID NO: 748.

21. The antibody of any one of claims 1 to 15, wherein the antibody comprises a combination of LCDR1, LCDR2, and LCDR3 and HCDR1, HCDR2, and HCDR3, wherein the combination is selected from the group consisting of:
(i) LCDR1 as set forth in SEQ ID NO: 13, LCDR2 as set forth in SEQ ID NO: 14, LCDR3 as set forth in SEQ ID NO: 15, HCDR1 as set forth in SEQ ID NO: 16, HCDR2 as set forth in SEQ ID NO: 17, HCDR3 as set forth in SEQ ID NO: 18;
(ii) LCDR1 as set forth in SEQ ID NO: 23, LCDR2 as set forth in SEQ ID NO: 24, LCDR3 as set forth in SEQ ID NO: 25, HCDR1 as set forth in SEQ ID NO: 26, HCDR2 as set forth in SEQ ID NO: 27, HCDR3 as set forth in SEQ ID NO: 28;
(iii) LCDR1 as set forth in SEQ ID NO: 33, LCDR2 as set forth in SEQ ID NO: 34, LCDR3 as set forth in SEQ ID NO: 35, HCDR1 as set forth in SEQ ID NO: 36, HCDR2 as set forth in SEQ ID NO: 37, HCDR3 as set forth in SEQ ID NO: 38;
(iv) LCDR1 as set forth in SEQ ID NO: 43, LCDR2 as set forth in SEQ ID NO: 44, LCDR3 as set forth in SEQ ID NO: 45, HCDR1 as set forth in SEQ ID NO: 46, HCDR2 as set forth in SEQ ID NO: 47, HCDR3 as set forth in SEQ ID NO: 48;
(v) LCDR1 as set forth in SEQ ID NO: 53, LCDR2 as set forth in SEQ ID NO: 54, LCDR3 as set forth in SEQ ID NO: 55, HCDR1 as set forth in SEQ ID NO: 56, HCDR2 as set forth in SEQ ID NO: 57, HCDR3 as set forth in SEQ ID NO: 58;
(vi) LCDR1 as set forth in SEQ ID NO: 63, LCDR2 as set forth in SEQ ID NO: 64, LCDR3 as set forth in SEQ ID NO: 65, HCDR1 as set forth in SEQ ID NO: 66, HCDR2 as set forth in SEQ ID NO: 67, HCDR3 as set forth in SEQ ID NO: 68;
(vii) LCDR1 as set forth in SEQ ID NO: 73, LCDR2 as set forth in SEQ ID NO: 74, LCDR3 as set forth in SEQ ID NO: 75, HCDR1 as set forth in SEQ ID NO: 76, HCDR2 as set forth in SEQ ID NO: 77, HCDR3 as set forth in SEQ ID NO: 78;
(viii) LCDR1 as set forth in SEQ ID NO: 83, LCDR2 as set forth in SEQ ID NO: 84, LCDR3 as set forth in SEQ ID NO: 85, HCDR1 as set forth in SEQ ID NO: 86, HCDR2 as set forth in SEQ ID NO: 87, HCDR3 as set forth in SEQ ID NO: 88;
(ix) LCDR1 as set forth in SEQ ID NO: 703, LCDR2 as set forth in SEQ ID NO: 704, LCDR3 as set forth in SEQ ID NO: 705, HCDR1 as set forth in SEQ ID NO: 706, HCDR2 as set forth in SEQ ID NO: 707, HCDR3 as set forth in SEQ ID NO: 708;
(x) LCDR1 as set forth in SEQ ID NO: 713, LCDR2 as set forth in SEQ ID NO: 714, LCDR3 as set forth in SEQ ID NO: 715, HCDR1 as set forth in SEQ ID NO: 716, HCDR2 as set forth in SEQ ID NO: 717, HCDR3 as set forth in SEQ ID NO: 718;
(xi) LCDR1 as set forth in SEQ ID NO: 723, LCDR2 as set forth in SEQ ID NO: 724, LCDR3 as set forth in SEQ ID NO: 725, HCDR1 as set forth in SEQ ID NO: 726, HCDR2 as set forth in SEQ ID NO: 727, HCDR3 as set forth in SEQ ID NO: 728;
(xii) LCDR1 as set forth in SEQ ID NO: 733, LCDR2 as set forth in SEQ ID NO: 734, LCDR3 as set forth in SEQ ID NO: 735, HCDR1 as set forth in SEQ ID NO: 736, HCDR2 as set forth in SEQ ID NO: 737, HCDR3 as set forth in SEQ ID NO: 738; and
(xiii) LCDR1 as set forth in SEQ ID NO: 743, LCDR2 as set forth in SEQ ID NO: 744, LCDR3 as set forth in SEQ ID NO: 745, HCDR1 as set forth in SEQ ID NO: 746, HCDR2 as set forth in SEQ ID NO: 747, HCDR3 as set forth in SEQ ID NO: 748.

22. The antibody of any one of claims 1 to 15, wherein the antibody comprises:
a. a light chain complementarity determining region 1 (LCDR1) comprising an amino acid sequence set forth in SEQ ID NO: 13;
b. a light chain complementarity determining region 2 (LCDR2) comprising an amino acid sequence set forth in SEQ ID NO: 14;
c. a light chain complementarity determining region 3 (LCDR3) comprising an amino acid sequence set forth in SEQ ID NO: 15;
d. a heavy chain complementarity determining region 1 (HCDR1) comprising an amino acid sequence set forth in SEQ ID NO: 16;
e. a heavy chain complementarity determining region 2 (HCDR2) comprising an amino acid sequence set forth in SEQ ID NO: 17; and/or
f. a heavy chain complementarity determining region 3 (HCDR3) comprising an amino acid sequence set forth in SEQ ID NO: 18.

23. The antibody of any one of claims 1 to 15, wherein the antibody comprises:
a. a light chain complementarity determining region 1 (LCDR1) comprising an amino acid sequence set forth in SEQ ID NO: 23;
b. a light chain complementarity determining region 2 (LCDR2) comprising an amino acid sequence set forth in SEQ ID NO: 24;
c. a light chain complementarity determining region 3 (LCDR3) comprising an amino acid sequence set forth in SEQ ID NO: 25;
d. a heavy chain complementarity determining region 1 (HCDR1) comprising an amino acid sequence set forth in SEQ ID NO: 26;
e. a heavy chain complementarity determining region 2 (HCDR2) comprising an amino acid sequence set forth in SEQ ID NO: 27; and/or
f. a heavy chain complementarity determining region 3 (HCDR3) comprising an amino acid sequence set forth in SEQ ID NO: 28.

24. The antibody of any one of claims 1 to 15, wherein the antibody comprises:
a. a light chain complementarity determining region 1 (LCDR1) comprising an amino acid sequence set forth in SEQ ID NO: 33;
b. a light chain complementarity determining region 2 (LCDR2) comprising an amino acid sequence set forth in SEQ ID NO: 34;
c. a light chain complementarity determining region 3 (LCDR3) comprising an amino acid sequence set forth in SEQ ID NO: 35;
d. a heavy chain complementarity determining region 1 (HCDR1) comprising an amino acid sequence set forth in SEQ ID NO: 36;
e. a heavy chain complementarity determining region 2 (HCDR2) comprising an amino acid sequence set forth in SEQ ID NO: 37; and/or
f. a heavy chain complementarity determining region 3 (HCDR3) comprising an amino acid sequence set forth in SEQ ID NO: 38.

25. The antibody of any one of claims 1 to 15, wherein the antibody comprises:
a. a light chain complementarity determining region 1 (LCDR1) comprising an amino acid sequence set forth in SEQ ID NO: 43;
b. a light chain complementarity determining region 2 (LCDR2) comprising an amino acid sequence set forth in SEQ ID NO: 44;
c. a light chain complementarity determining region 3 (LCDR3) comprising an amino acid sequence set forth in SEQ ID NO: 45;
d. a heavy chain complementarity determining region 1 (HCDR1) comprising an amino acid sequence set forth in SEQ ID NO: 46;
e. a heavy chain complementarity determining region 2 (HCDR2) comprising an amino acid sequence set forth in SEQ ID NO: 47; and/or
f. a heavy chain complementarity determining region 3 (HCDR3) comprising an amino acid sequence set forth in SEQ ID NO: 48.

26. The antibody of any one of claims 1 to 15, wherein the antibody comprises:
a. a light chain complementarity determining region 1 (LCDR1) comprising an amino acid sequence set forth in SEQ ID NO: 53;
b. a light chain complementarity determining region 2 (LCDR2) comprising an amino acid sequence set forth in SEQ ID NO: 54;
c. a light chain complementarity determining region 3 (LCDR3) comprising an amino acid sequence set forth in SEQ ID NO: 55;
d. a heavy chain complementarity determining region 1 (HCDR1) comprising an amino acid sequence set forth in SEQ ID NO: 56;
e. a heavy chain complementarity determining region 2 (HCDR2) comprising an amino acid sequence set forth in SEQ ID NO: 57; and/or
f. a heavy chain complementarity determining region 3 (HCDR3) comprising an amino acid sequence set forth in SEQ ID NO: 58.

27. The antibody of any one of claims 1 to 15, wherein the antibody comprises:
a. a light chain complementarity determining region 1 (LCDR1) comprising an amino acid sequence set forth in SEQ ID NO: 63;
b. a light chain complementarity determining region 2 (LCDR2) comprising an amino acid sequence set forth in SEQ ID NO: 64;
c. a light chain complementarity determining region 3 (LCDR3) comprising an amino acid sequence set forth in SEQ ID NO: 65;
d. a heavy chain complementarity determining region 1 (HCDR1) comprising an amino acid sequence set forth in SEQ ID NO: 66;
e. a heavy chain complementarity determining region 2 (HCDR2) comprising an amino acid sequence set forth in SEQ ID NO: 67; and/or
f. a heavy chain complementarity determining region 3 (HCDR3) comprising an amino acid sequence set forth in SEQ ID NO: 68.

28. The antibody of any one of claims 1 to 15, wherein the antibody comprises:
a. a light chain complementarity determining region 1 (LCDR1) comprising an amino acid sequence set forth in SEQ ID NO: 73;
b. a light chain complementarity determining region 2 (LCDR2) comprising an amino acid sequence set forth in SEQ ID NO: 74;
c. a light chain complementarity determining region 3 (LCDR3) comprising an amino acid sequence set forth in SEQ ID NO: 75;
d. a heavy chain complementarity determining region 1 (HCDR1) comprising an amino acid sequence set forth in SEQ ID NO: 76;
e. a heavy chain complementarity determining region 2 (HCDR2) comprising an amino acid sequence set forth in SEQ ID NO: 77; and/or
f. a heavy chain complementarity determining region 3 (HCDR3) comprising an amino acid sequence set forth in SEQ ID NO: 78.

29. The antibody of any one of claims 1 to 15, wherein the antibody comprises:
a. a light chain complementarity determining region 1 (LCDR1) comprising an amino acid sequence set forth in SEQ ID NO: 83;
b. a light chain complementarity determining region 2 (LCDR2) comprising an amino acid sequence set forth in SEQ ID NO: 84;
c. a light chain complementarity determining region 3 (LCDR3) comprising an amino acid sequence set forth in SEQ ID NO: 85;
d. a heavy chain complementarity determining region 1 (HCDR1) comprising an amino acid sequence set forth in SEQ ID NO: 86;
e. a heavy chain complementarity determining region 2 (HCDR2) comprising an amino acid sequence set forth in SEQ ID NO: 87; and/or
f. a heavy chain complementarity determining region 3 (HCDR3) comprising an amino acid sequence set forth in SEQ ID NO: 88.

30. The antibody of any one of claims 1 to 29, wherein the antibody comprises an immunoglobulin light chain amino acid variable region sequence at least about 90%, 95%, 96%, 97%, 98%, or 99% identical to any one of SEQ ID NOs: 155, 157, 169, 267, 271, 275, 277, 309, 311, 313, 315, 317, or 319; and an immunoglobulin heavy chain variable region amino acid sequence at least about 90%, 95%, 96%, 97%, 98%, or 99% identical to any one of SEQ ID NOs: 156, 158, 170, 268, 272, 276, 278, 310, 312, 314, 316, 318, or 320.

31. The antibody of any one of claims 1 to 29, wherein the antibody comprises an immunoglobulin light chain amino acid variable region sequence identical to any one of SEQ ID NOs: 155, 157, 169, 267, 271, 275, 277, 309, 311, 313, 315, 317, or 319; and an immunoglobulin heavyl chain variable region amino acid sequence identical to any one of SEQ ID NOs: 156, 158, 170, 268, 272, 276, 278, 310, 312, 314, 316, 318, or 320.

32. The antibody of any one of claims 1 to 15, wherein the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to any one of the Kabat definition, Chothia definition, Aho definition, Abm definition, IMGT definition, Contact definition and North definition.

33. The antibody of any one of claims 1 to 15, wherein the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined using a hybrid scheme of any two or three of the Kabat definition, Chothia definition, Aho definition, Abm definition, IMGT definition, Contact definition and North definition.

34. The antibody of any one of claims 1 to 15, wherein the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320; wherein the CDRs are defined according to the Kabat definition.

35. The antibody of any one of claims 1 to 15, wherein the antibody comprises: (i) a light chain variable region (VL) having the identical LCDR1, LCDR2, and LCDR3 with any one of SEQ ID NOs: 155, 157, 169, 267, 271, 275, 277, 309, 311, 313, 315, 317, and 319; and (ii) a heavy chain variable region (VH) having the identical HCDR1, HCDR2, and HCDR3 with any one of SEQ ID NOs: 156, 158, 170, 268, 272, 276, 278, 310, 312, 314, 316, 318, and 320;
wherein the CDRs are defined according to the Chothia definition.

36. The antibody of any one of claims 1 to 29, wherein the antibody comprises an immunoglobulin light chain variable region amino acid sequence at least about 90%, 95%, 96%, 97%, 98%, or 99% identical to SEQ ID NO: 275; and an immunoglobulin heavy chain variable region amino acid sequence at least about 90%, 95%, 96%, 97%, 98%, or 99% identical to SEQ ID NO: 276.

37. The antibody of any one of claims 1 to 29, wherein the antibody comprises an immunoglobulin light chain variable region amino acid sequence identical to SEQ ID NOs: 275;
and an immunoglobulin heavy chain variable region amino acid sequence identical to SEQ ID NO: 276.

38. The antibody of any one of claims 1 to 29, wherein the antibody comprises an immunoglobulin light chain variable region amino acid sequence at least about 90%, 95%, 96%, 97%, 98%, or 99% identical to SEQ ID NO: 277; and an immunoglobulin heavy chain variable region amino acid sequence at least about 90%, 95%, 96%, 97%, 98%, or 99% identical to SEQ ID NO: 278.

39. The antibody of any one of claims 1 to 29, wherein the antibody comprises an immunoglobulin light chain variable region amino acid sequence identical to SEQ ID NOs: 277; and an immunoglobulin heavy chain variable region amino acid sequence identical to SEQ ID NO: 278.

40. The antibody of any one of claims 1 to 29, wherein the antibody comprises an immunoglobulin light chain variable region amino acid sequence at least about 90%, 95%, 96%, 97%, 98%, or 99% identical to SEQ ID NO: 309; and an immunoglobulin heavy chain variable region amino acid sequence at least about 90%, 95%, 96%, 97%, 98%, or 99% identical to SEQ ID NO: 310.

41. The antibody of any one of claims 1 to 29, wherein the antibody comprises an immunoglobulin light chain variable region amino acid sequence identical to SEQ ID NOs: 309; and an immunoglobulin heavy chain variable region amino acid sequence identical to SEQ ID NO: 310.

42. The antibody of any one of claims 1 to 29, wherein the antibody comprises an immunoglobulin light chain variable region amino acid sequence at least about 90%, 95%, 96%, 97%, 98%, or 99% identical to SEQ ID NO: 155; and an immunoglobulin heavy chain variable region amino acid sequence at least about 90%, 95%, 96%, 97%, 98%, or 99% identical to SEQ ID NO: 156.

43. The antibody of any one of claims 1 to 29, wherein the antibody comprises an immunoglobulin light chain variable region amino acid sequence identical to SEQ ID NOs: 155; and an immunoglobulin heavy chain variable region amino acid sequence identical to SEQ ID NO: 156.

44. The antibody of any one of claims 1 to 29, wherein the antibody comprises an immunoglobulin light chain variable region amino acid sequence at least about 90%, 95%, 96%, 97%, 98%, or 99% identical to SEQ ID NO: 157; and an immunoglobulin heavy chain variable region amino acid sequence at least about 90%, 95%, 96%, 97%, 98%, or 99% identical to SEQ ID NO: 158.

45. The antibody of any one of claims 1 to 29, wherein the antibody comprises an immunoglobulin light chain variable region amino acid sequence identical to SEQ ID NOs: 157; and an immunoglobulin heavy chain variable region amino acid sequence identical to SEQ ID NO: 158.

46. The antibody of any one of claims 1 to 29, wherein the antibody comprises an immunoglobulin light chain variable region amino acid sequence at least about 90%, 95%, 96%, 97%, 98%, or 99% identical to SEQ ID NO: 169; and an immunoglobulin heavy chain variable region amino acid sequence at least about 90%, 95%, 96%, 97%, 98%, or 99% identical to SEQ ID NO: 170.

47. The antibody of any one of claims 1 to 29, wherein the antibody comprises an immunoglobulin light chain variable region amino acid sequence identical to SEQ ID NOs: 169; and an immunoglobulin heavy chain variable region amino acid sequence identical to SEQ ID NO: 170.

48. The antibody of any one of claims 1 to 29, wherein the antibody comprises an immunoglobulin light chain variable region amino acid sequence at least about 90%, 95%, 96%, 97%, 98%, or 99% identical to SEQ ID NO: 267; and an immunoglobulin heavy chain variable region amino acid sequence at least about 90%, 95%, 96%, 97%, 98%, or 99% identical to SEQ ID NO: 268.

49. The antibody of any one of claims 1 to 29, wherein the antibody comprises an immunoglobulin light chain variable region amino acid sequence identical to SEQ ID NOs: 267; and an immunoglobulin heavy chain variable region amino acid sequence identical to SEQ ID NO: 268.

50. The antibody of any one of claims 1 to 29, wherein the antibody comprises an immunoglobulin light chain variable region amino acid sequence at least about 90%, 95%, 96%, 97%, 98%, or 99% identical to SEQ ID NO: 271; and an immunoglobulin heavy chain variable region amino acid sequence at least about 90%, 95%, 96%, 97%, 98%, or 99% identical to SEQ ID NO: 272.

51. The antibody of any one of claims 1 to 29, wherein the antibody comprises an immunoglobulin light chain variable region amino acid sequence identical to SEQ ID NOs: 271; and an immunoglobulin heavy chain variable region amino acid sequence identical to SEQ ID NO: 272.

52. The antibody of any one of claims 1 to 29, wherein the antibody comprises an immunoglobulin light chain variable region amino acid sequence at least about 90%, 95%, 96%, 97%, 98%, or 99% identical to any one of the immunoglobulin light chain variable region sequences of Table 1; and an immunoglobulin heavy chain variable region amino acid sequence at least about 90%, 95%, 96%, 97%, 98%, or 99% identical to any one of the immunoglobulin heavy chain variable region sequences of Table 1.

53. The antibody of any one of claims 1 to 29, wherein the antibody comprises an immunoglobulin light chain amino acid sequence identical to any one of the immunoglobulin light chain variable region sequences of Table 1; and an immunoglobulin heavy chain variable region amino acid sequences identical to any one of the immunoglobulin heavy chain variable region sequences of Table 1.

54. The antibody of any one of claims 1 to 53, wherein the antibody comprises an arginine substitution at position number 17 of the immunoglobulin light chain according to Kabat numbering.

55. The antibody of any one of claims 1 to 54, wherein the antibody comprises a tyrosine substitution at position number 12 of the immunoglobulin light chain according to Kabat numbering.

56. The antibody of any one of claims 1 to 55, wherein the antibody does not bind to T lymphocytes.

57. The antibody of any one of claims 1 to 56, wherein the antibody does not bind to activated T lymphocytes.

58. The antibody of any one of claims 1 to 57, wherein the antibody does not bind to B lymphocytes.

59. The antibody of any one of claims 1 to 58, wherein the antibody comprises an Fc region comprising an amino acid alteration selected from the list consisting of D356E, L358M, and combinations thereof.

60. A pharmaceutical composition comprising the antibody of any one of claims 1 to 59 and a pharmaceutically acceptable carrier, diluent, or excipient.

61. The pharmaceutical composition of claim 60 formulated for intravenous administration.

62. A nucleic acid encoding the antibody of any one of claims 1 to 59.

63. An expression vector comprising the nucleic acid of claim 62.

64. A eukaryotic cell comprising the expression vector of claim 63.

65. The eukaryotic cell of claim 64, wherein the eukaryotic cell is a CHO cell line.

66. A method of treating a cancer or tumor comprising administering to an individual afflicted with the cancer or tumor the antibody of any one of claims 1 to 59 or the pharmaceutical composition of claim 60 or 61.

67. The method of claim 66, wherein the cancer or tumor is a solid tissue cancer.

68. The method of claim 66, wherein the cancer or tumor is a blood cancer.

69. The method of claim 66, wherein the cancer or tumor comprises breast cancer, colorectal cancer, or ovarian cancer.

70. The method of claim 69, wherein the breast cancer is triple negative breast cancer.

71. A method of inhibiting CD24 signaling in a cancer cell comprising contacting the cancer cell to the antibody of any one of claims 1 to 59 or the pharmaceutical composition of claim 60 or 61.

72. The method of claim 71, wherein CD24 signaling is not inhibited in non-neoplastic B lymphocytes or non-neoplastic T lymphocytes.

73. The method of claim 71 or 72, wherein the cancer cell is a cancer cell is in vivo in an individual.
